SEQUENCE LISTING (106 sequences)

SEQ ID NO: 1 - protein, 344 aa, Diatraea saccharalis
MAPNLIKNEGTFDVDIKPDELQLPKLSNIKPKILHVNLIYFLYWHLAAPYGLYLCCTSAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAIPVFASFCYILPTLIPMYLWGESFNNSWHVNLLRYIMNLNVTLLVNSAAHRWGYKTYEKNINPTQNVYVSFVTLGEGFHNYHHIFPWDYKTAELGNNKLNVTTWFINFFANIGWAYDLKTVSNEMARARAMRTGDGKDLWGWDEKDVTDKDKEHTGILNGKTD

SEQ ID NO: 2 - DNA, 1034 bp, Diatraea saccharalis
  source 1..1034: mol_type = other DNA
atggctcccaacctgatcaagaacgagggcaccttcgacgtggacatcaagcccgacgaactgcagctgcccaagctgtctaacattaagcccaagatcctgcacgtgaacctgatctacttcctgtactggcacctggctgctccctacggcctgtacctgtgctgcacctctgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccagaagaagtacgctatccccgttttcgcctctttctgctacattctgcccactctgatccccatgtacctctggggcgagtctttcaacaactcttggcacgtcaacctgctgcggtacatcatgaacctgaacgtgaccctgctggtgaactctgccgctcaccgatggggctacaagacctacgaaaagaacatcaaccccactcagaacgtgtacgtgtctttcgtgaccctcggcgagggcttccacaactaccaccacatctttccatgggattacaagaccgctgagctgggaaacaacaagctgaacgtcactacctggttcatcaactttttcgccaacatcggctgggcctacgacctcaagaccgtgtctaacgagatggcccgagccagagccatgcgaaccggcgacggcaaggacctgtggggctgggacgagaaggacgtgaccgacaaggataaggaacacaccggcatcctgaacggcaagaccgacta

SEQ ID NO: 3 - protein, 320 aa, Dendrolimus punctatus
MAPNITNEEIIVSETDHDQSKRPQAEPRQYHIVYRNIAVFLYYHIAGIYGLYLCFGNAMWTTIFWNVFTFIIAGLGVTAGAHRLWSHKAYKATRPLQIFLVLAQSVANQHSVAHWARDHRLHHKYSETDADPHNSTRGFFYSHVGWLMVRKHPEVTRRGKTVDMSDIYNNPYLKLQDKHSYWFIPLLSFFIPTYIPTLWGESLTNAWHINMFRYILNLNATFCVNSVAHLWGQKPYDKHIAPSQSPKVNVVTLGEGFHNYHHCFPWDYRSDELGLTFNFTTGFIDICEKLGLAYDLRSASEEVIAARAERTGDGSRIKSL

SEQ ID NO: 4 - DNA, 963 bp, Dendrolimus punctatus
  source 1..963: mol_type = other DNA
atggctcccaacatcaccaacgaagagatcatcgtgtctgagactgaccacgaccagtctaagcgaccccaggctgagccccgacagtaccacatcgtgtaccgaaacattgccgtgtttctgtactaccacattgccggcatctacggcctgtacctgtgcttcggcaacgctatgtggaccaccattttctggaacgtgttcaccttcattatcgccggcctgggcgtgaccgctggcgcccaccgactgtggtcccacaaggcctacaaggccactcgacccctgcagatcttcctggtgctggcccagtctgtggccaaccagcactctgtggcccactgggcccgagatcaccgactgcaccacaagtactccgagactgacgctgaccctcacaactctacccgaggcttcttctactctcacgtcggctggctgatggtgcgaaagcaccccgaggtgacccgacgaggcaagaccgtggacatgtctgacatctacaacaacccctacctgaagctgcaggacaagcactcttactggttcatccctctgctgtctttcttcatccccacctacattcctactctgtggggcgagtctctgaccaacgcctggcacatcaacatgttccggtacatcctgaacctgaacgccaccttctgcgtgaactccgtggctcacctgtggggacagaagccctacgacaagcacattgctccctcgcagtctcccaaggtgaacgtggtgaccctcggcgagggcttccacaactaccaccactgcttcccttgggactaccgatctgacgagctgggcctgaccttcaacttcaccaccggcttcatcgacatctgcgagaagctcggcctggcctacgacctgcgatctgcctctgaggaagtgatcgccgctcgagccgagcgaaccggcgacggctctcgaatcaagtctctgtag

SEQ ID NO: 5 - DNA, 1149 bp, Drosophila grimshawi
  source 1..1149: mol_type = other DNA
atgcctccaaacgctatctcttctcctcctgacgctctgcccacctgtcctgaggaccaggtcctgtctggctctcagtctggcgctggcggagcctctaagcagaccggcgtgctgttcgaggccgacgccgacaccaacgacggcggcctgaccatggacatcacccagttcaagaacgccgagaagcgaaagctgaagtgggtgtggcgaaacatcgtcctgttcgcctacgtccacgtggccgctctgtacggcggctacctgctggtgacccaggccaagtgggccactatcgtgttctctatgttcctgtactctgccggcatgatcggcatcaccggcggagcccaccgactgtgggctcaccgatcttacaaggctaagtggcccctgcgagtgatcctggtggccttcaactctatcgccttccaggacgccgcctaccactgggcccgagatcaccgagtgcaccacaagttctctgagactgacgctgaccctcacaacgctacccgaggcttcttcttctctcacgtcggctggctgctgtgcaagaagcaccccgacgtcgtcgccaagggcaagtctctggacctgtctgacctgcgagccgaccgagtgctgatgttccaaaagaagcactacttcgttctgatgcccctggcctgcttcattctgcctacctgcattcccatgctgtgctggaacgagtctctgctgtgctctgtgctggtgcccaccatcttccgatggtgcctgcagctgaacatgacctggctggtgaactctgccgctcacaagttcggcggacgaccctacgacaagaacatcaacccttctcagtctccctacgtgtctctgttcaccctcggcgaaggctggcacaactaccaccacgtgttcccttgggactacaagaccgccgagtggggcaactactctctcaacatgaccaccgccttcattgacctgttcgctaagatcggctgggcctacgacctgaagtctgtggtgcccgagactgtcgagcgacgagtgcgacgaaccggcgacggctctcacgagctgtggggctggggcgacaaggacctgacacctgaggacggccagtctgtgctgcacgtgctggacaagaagtctaactag

SEQ ID NO: 6 - DNA, 1146 bp, Drosophila virilis
  source 1..1146: mol_type = other DNA
atgcctccaaacgccatcacttctccctctaacgtgctggccacctgtcctgaggtcgagcagctgccctctggcgagtgcgcctctaccgcctcttctaagcagaccggcgtgctgttcgagggcgacgccgacaccgccgactgcgccctggacgtggacgtgaagaagctgaagaaggccgagaagcgaaagctgaagctggtgtggcgaaacatcatgctgttcggctacctgcacctggccgccgtgtacggcggatacctgatgctgacccaggccaagtgggctaccgtggtgttctctttcttcctgtacaccgccggcatgatcggcatcaccggcggagcccaccgactgtgggctcaccgatcttacaaggctaagtggcccctgcgagtgatcctggtgaccttcaacaccattgccttccaggacgccgccttccactgggcccgagatcaccgagtgcaccacaagttctctgagactgacgctgaccctcacaacgctacccgaggcttcttcttctctcacgtcggctggctgctgtgcaagaagcaccccgacgtcgtcgccaagggcaagtctctggacgtgaccgacctgcgagccgaccgaatcctgatgttccagctgaagcactacttcgtgctgatgcccctggcctgcttcattctgcccaccatcattcccatgatctgctggaacgagtctctgctgtgctcctggttcgtggccaccatgttccgatggtgcttccagctcaacatgacctggctggtgaactctgccgctcacaagttcggcggacgaccctacgacaagaacatcaacccctctcagtctccctacgtgtctgccttcaccttcggcgaaggctggcacaactaccaccacgtgttcccttgggactacaagaccgccgagtggggccgatactctctgaacatgactaccgccttcatcgactttttcgccaagatcggctgggcctacgagctgaagtctgtggctcccgagactatcgagcgacgagtgcgacgaaccggcgacggcacccacgagctgtggggctggggcgacaaggacctgaccgctgaggacgcccagcacgtcctgttcgtggacaagaaggctgcctag

SEQ ID NO: 7 - DNA, 1086 bp, Drosophila melanogaster
  source 1..1086: mol_type = other DNA
atggctccctactctcgaatctaccaccaggacaagtcgtcccgagagactggcgtgctgttcgaggacgacgcccagaccgtggactctgacctgaccaccgaccgattccagctgaagcgagccgagaagcgacgactgcccctggtgtggcgaaacatcatcctgttcgccctggtgcacctggccgctctgtacggcctgcactctatcttcacccgagccaagctggccaccactctgttcgctgccggcctgtacatcatcggcatgctgggcgtgaccgctggcgcccaccgactgtgggctcaccgaacctacaaggccaagtggcccctgcgactgctgctggtgatcttcaacaccattgccttccaggacgccgtgtaccactgggcccgagatcaccgagtgcaccacaagtactctgagactgacgctgaccctcacaacgctacccgaggcttcttcttctctcacgtcggctggctgctgtgcaagaagcaccccgacatcaaggaaaagggccgaggcctggacctgtctgacctgcgagctgaccccatcctgatgttccagcgaaagcactactacattctgatgcccctggcctgcttcgtgctgcccaccgtgattcccatggtgtactggaacgagactctggcctcttcctggttcgtggccaccatgttccgatggtgcttccagctcaacatgacctggctggtgaactctgccgctcacaagttcggcaaccgaccttacgacaagactatgaaccccactcagaacgccttcgtgtctgccttcaccttcggcgaaggctggcacaactaccaccacgcattcccttgggactacaagaccgccgagtggggctgctactctctgaacatcaccaccgccttcatcgacctgttcgctaagatcggctgggcctacgacctcaagaccgtggctcccgacgtgatccagcgacgagtgctgcgaaccggcgacggctctcacgagctgtggggctggggcgacaaggacctgaccgctgaggacgcccgaaacgtcctgctggtggacaagtctcgataa

SEQ ID NO: 8 - DNA, 1449 bp, Yarrowia lipolytica
  source 1..1449: mol_type = other DNA
atggtgaaaaacgtggaccaagtggatctctcgcaggtcgacaccattgcctccggccgagatgtcaactacaaggtcaagtacacctccggcgttaagatgagccagggcgcctacgacgacaagggccgccacatttccgagcagcccttcacctgggccaactggcaccagcacatcaactggctcaacttcattctggtgattgcgctgcctctgtcgtcctttgctgccgctcccttcgtctccttcaactggaagaccgccgcgtttgctgtcggctattacatgtgcaccggtctcggtatcaccgccggctaccaccgaatgtgggcccatcgagcctacaaggccgctctgcccgttcgaatcatccttgctctgtttggaggaggagctgtcgagggctccatccgatggtgggcctcgtctcaccgagtccaccaccgatggaccgactccaacaaggacccttacgacgcccgaaagggattctggttctcccactttggctggatgctgcttgtgcccaaccccaagaacaagggccgaactgacatttctgacctcaacaacgactgggttgtccgactccagcacaagtactacgtttacgttctcgtcttcatggccattgttctgcccaccctcgtctgtggctttggctggggcgactggaagggaggtcttgtctacgccggtatcatgcgatacacctttgtgcagcaggtgactttctgtgtcaactcccttgcccactggattggagagcagcccttcgacgaccgacgaactccccgagaccacgctcttaccgccctggtcacctttggagagggctaccacaacttccaccacgagttcccctcggactaccgaaacgccctcatctggtaccagtacgaccccaccaagtggctcatctggaccctcaagcaggttggtctcgcctgggacctccagaccttctcccagaacgccatcgagcagggtctcgtgcagcagcgacagaagaagctggacaagtggcgaaacaacctcaactggggtatccccattgagcagctgcctgtcattgagtttgaggagttccaagagcaggccaagacccgagatctggttctcatttctggcattgtccacgacgtgtctgcctttgtcgagcaccaccctggtggaaaggccctcattatgagcgccgtcggcaaggacggtaccgctgtcttcaacggaggtgtctaccgacactccaacgctggccacaacctgcttgccaccatgcgagtttcggtcattcgaggcggcatggaggttgaggtgtggaagactgcccagaacgaaaagaaggaccagaacattgtctccgatgagagtggaaaccgaatccaccgagctggtctccaggccacccgggtcgagaaccccggtatgtctggcatggctgcttag

SEQ ID NO: 9 - DNA, 963 bp, Tribolium castaneum
  source 1..963: mol_type = other DNA
atgtctaccctcgagactcaggtgcagatcgtgtggcgaaacgtgatcctgttcatctacctgcacgtggccgccatctacggcctgtacttcaccttcaccgccgccaagtggcccactatcctgtttacctacttcctgaccatcatctctacccaaggcaccggcgctggcgtgcaccgactgtggtcccaccgatcttacaaggccaagctgcccctgcgaatcctgctgtgcatctaccagactcactgcctgcagaaccacatctacgagtgggtgcgagatcatcgagcccaccacaagttctctgacaccgacgctgaccctcacaactctacccgaggcttcttcttctctcacatgggctggctgctggtgcgaaagcaccctcaggtgaagattaagggcaagctgatcgacctgtctgacctggaagaggaccaggtggtgatgttccagaagaagtactacctgattctggctcccttcttcgccttcctgctgcctgcctgggtgccctggtacttctggggcgaagatctgcacgtgtcttggtgcgtggcctctatgctgcgatacgccctgtctctgcacggcacctggctggtgaactctgccgctcacatgtggggcacccgaccttacgaccgaaacatcaaggccaccgagactaaggtggtgtcttacatcaccaacggcgagcgattccacaactaccaccacacattcccttgggactacaaggccgccgagctgggctcttactggggcaactggtctaccgccttcatcgacttcatggccaagatcggctgggcctacgacctgaagatcatccctcctgagctggtcgagaagcgagccaagcgaaccggcgagtgcacccacaaggtgtggggctggggcgacaaggacatcgacaaggaagagatcgagattgtcgagcgaaaccgaggctaa

SEQ ID NO: 10 - DNA, 1056 bp, Bombus lapidarius
  source 1..1056: mol_type = other DNA
atggctcccaacatcacctcttctcccaccggcgtgctgttcgagggcgagactctggaagagacttctcgagtgatcgacgctcccaagaccaagttcaagacccagatcgtgtggcgaaacgtgatcatcttcacctacctgcacatctctgccgtgtacggcctgtacctggccttcacctctgccaagtgggctaccctgctgttcgccttcttcctgcacgtgtactctgccctgggcatcaccgctggcgcccaccgactgtgggctcaccgatcttacaaggccaagtggcccatgcagctgatcctgatcatcggcaacaccattgccttccaggactctgccatcgactgggcccgagatcaccgactgcaccacaagtactctgagactaacgctgaccctcacaacgccaagcgaggcttcttcttcgcccacatcggctggctgctgtgcaagaagcaccccgacatccgagccaagggcaagggcatcgacatgtctgacctgaagaacaaccccatcctgaccttccagaagaagtactacaccattctgatgcccctgctgtgcttcatcgtgcccaccatgattcccgtgtactgctggaacgaaacctggggcaacggctacttcatccccgccatcctgcgatacgtgtacaccctgaacatgacctggctggtgaactctgccgctcacatgttcggcaacaagccctacgacaagtacatcaaccccgtcgagaacaagatggtggccattaccgctctcggcgaaggctggcacaactaccaccacgtgttcccttgggactacaagaccgccgagctgggcaactacaaggtgaacgtgaccactctgttcatcgacgtgtgctctaagctgggcctcgcctacgacatgaagatcgtgccccaggacctggtgcagaagcgagtcgagcgaaccggcgacggctctcacaacgtgtggggctggggcgacaaggaccagactcagcaggaccgagatgtgaccatggtggtcaacctcaagaaggaccactag

SEQ ID NO: 11 - DNA, 1059 bp, Lobesia botrana
  source 1..1059: mol_type = other DNA
atgcctccaaacgtgaccgacgtgaacggcgtgctgttcgagtctgacgccgctactcccgacctggctctggctacccctcctgtgcagaaggccgacaacacccctcgagtctacgtgtggcgaaacatcatcctgttcggctacctgcacattgccgctctgtacggcggatacctgttcctggtgtctgctaagtggcagaccgacatcttcgcctacctgctgtacgtggcctctggcctgggcatcaccgctggcgcccaccgactgtgggctcacaagtcttacaaggccaagtggcccctgcgagtgatcctgaccatcttcaacaccattgccttccaggactctgccatcgactgggcccgagatcaccgaatgcaccacaagtactctgagactgacgctgaccctcacaacgctacccgaggcttcttcttctctcacatcggctggctgctggtgcgaaagcaccccgagctgaagcgaaagggcaagggcctcgacctgtctgacctgtacgctgatcccatcctgcgattccagaagaagtactacctgattctgatgcccctggcctgcttcatcatgcccactctgatccccgtgtacttctggggcgagacttggaccaacgccttcttcgtggctgccctgttccgatacgccttcatcctgaacatcacctggctggtgaactctgctgcccacaagtggggcgacaagccctacgacaagaccatcaagccctctgagaacctgtctgtgtctctgttcgccttcggcgagggcttccacaactaccaccacacattcccttgggactacaagaccgccgagctgggcaaccaccgactcaacttcactaccaagttcatcaactttttcgccaagctcggctgggcctacgacatgaagaccgtgtctgacgagatcgtgcagaagcgagtccagcgaaccggcgacggctctcaccacctgtggggctggggagacaaggaccagtctaaggacgaggtggacgccgccatccgaatcaaccccaaggacgactag

SEQ ID NO: 12 - DNA, 1533 bp, Saccharomyces cerevisiae
  source 1..1533: mol_type = other DNA
atgcccacctctggcaccaccatcgagctgattgacgatcagttccccaaggacgactctgcctcttctggcatcgtggacgaggtggacctgaccgaggccaacatcctggccaccggcctgaacaagaaggctccccgaatcgtcaacggcttcggctctctgatgggctctaaggaaatggtgtctgtcgagttcgacaagaagggcaacgagaagaagtctaacctggaccgactgctcgagaaggacaaccaggaaaaggaagaggccaagaccaagatccacatctctgagcagccctggactctgaacaactggcaccagcacctgaactggctgaacatggtgctggtctgcggcatgcccatgatcggctggtacttcgccctgtctggcaaggtgcccctgcacctcaacgtgtttctgttctctgtgttctactacgccgtcggcggcgtgtctatcaccgccggctaccaccgactgtggtcccaccgatcttactctgctcactggcccctgcgactgttctacgccatcttcggctgcgcctctgtcgagggctctgccaagtggtggggccactctcaccgaatccaccaccgatacaccgacactctgcgagatccctacgacgcccgacgaggcctgtggtactctcacatgggctggatgctgctgaagcccaaccctaagtacaaggcccgagccgacatcaccgacatgactgacgactggaccatccgattccagcaccgacattacatcctgctgatgctgctcaccgccttcgtgatccccactctgatctgcggctacttcttcaacgactacatgggcggcctgatctacgccggcttcatccgagtgttcgtgattcagcaggctaccttctgtatcaactctctggcccactacatcggcacccagcctttcgacgaccgacgaacccctcgagacaactggatcaccgccattgtgaccttcggcgagggctaccacaacttccaccacgagttccccaccgactaccgaaacgccatcaagtggtatcagtacgaccccaccaaggtgattatctacctgacctctctggtcggcctggcctacgacctgaagaagttctctcagaacgccatcgaggaagccctgatccagcaagagcagaagaagatcaacaagaagaaggcaaagatcaactggggacccgtgctgaccgacctgcctatgtgggacaagcagaccttcctggccaagtccaaggaaaacaagggcctcgtcatcatctctggcattgtgcacgacgtgtctggctacatttctgagcaccccggtggcgagactctgatcaagaccgctctcggcaaggacgccaccaaggccttctctggcggagtgtaccgacattctaacgccgctcagaacgtgctggccgacatgcgagtggccgtgatcaaggaatctaagaactctgccatccgaatggcctctaagcgaggcgagatctacgagactggcaagttcttttag

SEQ ID NO: 13 - DNA, 1062 bp, Spodoptera litura
  source 1..1062: mol_type = other DNA
atggctcccaacatctctgaggacgtgaacggcgtgctgttcgagtctgacgccgctactcccgacctggctctggcccgacctcctgtgcagaaggccgacaacaagcccaagcagctggtgtggcgaaacatcatcctgttcgcctacctgcacctggccgctctgtacggcggctacctgttcctgttctctgccaagtggcagaccgacatcttcgcctacatcctgtacgtgatctctggcctgggcatcaccgctggcgcccaccgactgtgggctcacaagtcttacaaggctaagtggcccctgaaggtgatcctgatcatcttcaacaccgtggccttccaggacgccgccatggactgggcccgagatcaccgaatgcaccacaagtactctgagactgacgctgaccctcacaacgctacccgaggcttcttcttctctcacatcggctggctgctggtgcgaaagcaccccgacctgaaggaaaagggcaagggcctcgacatgtctgacctgctggctgaccccgtgctgcgattccagaagaagtactacctgctgctgatgcccctggcctgcttcgtcatgcccaccgtgattcccgtgtacctgtggggcgagacttggaccaacgccttcttcgtggccgccatgttccgatacgccttcattctgaacgtgacctggctggtgaactctgctgcccacaagtggggcgacaagccctacgacaagtctatcaagccctctgagaacatgtctgtggccatgttcgccctcggcgagggattccacaactaccaccacacattcccttgggactacaagaccgccgagttcggcaacaacaagctgaacttcactaccgccttcatcaactttttcgccaagatcggctgggcttacgacatgaagaccgtgtctgaggacatcgtgaagaaccgagtgaagcgaaccggcgacggctctcaccacctctggggctggggcgacgagaaccagcctaaggaagagatcgaggccgccatccgaatcaaccccaaggacgactaa

SEQ ID NO: 14 - protein, 416 aa, Drosophila grimshawi
MFSEYYVYRRKLLGISSVQIARQIDRLIKPSTLKMPPNAISSPPDALPTCPEDQVLSGSQSGAGGASKQTGVLFEADADTNDGGLTMDITQFKNAEKRKLKWVWRNIVLFAYVHVAALYGGYLLVTQAKWATIVFSMFLYSAGMIGITGGAHRLWAHRSYKAKWPLRVILVAFNSIAFQDAAYHWARDHRVHHKFSETDADPHNATRGFFFSHVGWLLCKKHPDVVAKGKSLDLSDLRADRVLMFQKKHYFVLMPLACFILPTCIPMLCWNESLLCSVLVPTIFRWCLQLNMTWLVNSAAHKFGGRPYDKNINPSQSPYVSLFTLGEGWHNYHHVFPWDYKTAEWGNYSLNMTTAFIDLFAKIGWAYDLKSVVPETVERRVRRTGDGSHELWGWGDKDLTPEDGQSVLHVLDKKSN

SEQ ID NO: 15 - protein, 381 aa, Drosophila virilis
MPPNAITSPSNVLATCPEVEQLPSGECASTASSKQTGVLFEGDADTADCALDVDVKKLKKAEKRKLKLVWRNIMLFGYLHLAAVYGGYLMLTQAKWATVVFSFFLYTAGMIGITGGAHRLWAHRSYKAKWPLRVILVTFNTIAFQDAAFHWARDHRVHHKFSETDADPHNATRGFFFSHVGWLLCKKHPDVVAKGKSLDVTDLRADRILMFQLKHYFVLMPLACFILPTIIPMICWNESLLCSWFVATMFRWCFQLNMTWLVNSAAHKFGGRPYDKNINPSQSPYVSAFTFGEGWHNYHHVFPWDYKTAEWGRYSLNMTTAFIDFFAKIGWAYELKSVAPETIERRVRRTGDGTHELWGWGDKDLTAEDAQHVLFVDKKAA

SEQ ID NO: 16 - protein, 361 aa, Drosophila melanogaster
MAPYSRIYHQDKSSRETGVLFEDDAQTVDSDLTTDRFQLKRAEKRRLPLVWRNIILFALVHLAALYGLHSIFTRAKLATTLFAAGLYIIGMLGVTAGAHRLWAHRTYKAKWPLRLLLVIFNTIAFQDAVYHWARDHRVHHKYSETDADPHNATRGFFFSHVGWLLCKKHPDIKEKGRGLDLSDLRADPILMFQRKHYYILMPLACFVLPTVIPMVYWNETLASSWFVATMFRWCFQLNMTWLVNSAAHKFGNRPYDKTMNPTQNAFVSAFTFGEGWHNYHHAFPWDYKTAEWGCYSLNITTAFIDLFAKIGWAYDLKTVAPDVIQRRVLRTGDGSHELWGWGDKDLTAEDARNVLLVDKSR

SEQ ID NO: 17 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 18 - protein, 320 aa, Tribolium castaneum
MSTLETQVQIVWRNVILFIYLHVAAIYGLYFTFTAAKWPTILFTYFLTIISTQGTGAGVHRLWSHRSYKAKLPLRILLCIYQTHCLQNHIYEWVRDHRAHHKFSDTDADPHNSTRGFFFSHMGWLLVRKHPQVKIKGKLIDLSDLEEDQVVMFQKKYYLILAPFFAFLLPAWVPWYFWGEDLHVSWCVASMLRYALSLHGTWLVNSAAHMWGTRPYDRNIKATETKVVSYITNGERFHNYHHTFPWDYKAAELGSYWGNWSTAFIDFMAKIGWAYDLKIIPPELVEKRAKRTGECTHKVWGWGDKDIDKEEIEIVERNRG

SEQ ID NO: 19 - protein, 351 aa, Bombus lapidarius
MAPNITSSPTGVLFEGETLEETSRVIDAPKTKFKTQIVWRNVIIFTYLHISAVYGLYLAFTSAKWATLLFAFFLHVYSALGITAGAHRLWAHRSYKAKWPMQLILIIGNTIAFQDSAIDWARDHRLHHKYSETNADPHNAKRGFFFAHIGWLLCKKHPDIRAKGKGIDMSDLKNNPILTFQKKYYTILMPLLCFIVPTMIPVYCWNETWGNGYFIPAILRYVYTLNMTWLVNSAAHMFGNKPYDKYINPVENKMVAITALGEGWHNYHHVFPWDYKTAELGNYKVNVTTLFIDVCSKLGLAYDMKIVPQDLVQKRVERTGDGSHNVWGWGDKDQTQQDRDVTMVVNLKKDH

SEQ ID NO: 20 - protein, 352 aa, Lobesia botrana
MPPNVTDVNGVLFESDAATPDLALATPPVQKADNTPRVYVWRNIILFGYLHIAALYGGYLFLVSAKWQTDIFAYLLYVASGLGITAGAHRLWAHKSYKAKWPLRVILTIFNTIAFQDSAIDWARDHRMHHKYSETDADPHNATRGFFFSHIGWLLVRKHPELKRKGKGLDLSDLYADPILRFQKKYYLILMPLACFIMPTLIPVYFWGETWTNAFFVAALFRYAFILNITWLVNSAAHKWGDKPYDKTIKPSENLSVSLFAFGEGFHNYHHTFPWDYKTAELGNHRLNFTTKFINFFAKLGWAYDMKTVSDEIVQKRVQRTGDGSHHLWGWGDKDQSKDEVDAAIRINPKDD

SEQ ID NO: 21 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSLAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

SEQ ID NO: 22 - protein, 353 aa, Spodoptera litura
MAPNISEDVNGVLFESDAATPDLALARPPVQKADNKPKQLVWRNIILFAYLHLAALYGGYLFLFSAKWQTDIFAYILYVISGLGITAGAHRLWAHKSYKAKWPLKVILIIFNTVAFQDAAMDWARDHRMHHKYSETDADPHNATRGFFFSHIGWLLVRKHPDLKEKGKGLDMSDLLADPVLRFQKKYYLLLMPLACFVMPTVIPVYLWGETWTNAFFVAAMFRYAFILNVTWLVNSAAHKWGDKPYDKSIKPSENMSVAMFALGEGFHNYHHTFPWDYKTAEFGNNKLNFTTAFINFFAKIGWAYDMKTVSEDIVKNRVKRTGDGSHHLWGWGDENQPKEEIEAAIRINPKDD

SEQ ID NO: 23 - DNA, 25 bp, synthetic construct
  misc_feature 1..25: note = Artificial
  source 1..25: mol_type = other DNA
  modified_base 8: mod_base = OTHER; note = Uracil
cgtgcgatacagaccgggttggcgg

SEQ ID NO: 24 - DNA, 55 bp, synthetic construct
  misc_feature 1..55: note = Artificial
  source 1..55: mol_type = other DNA
  modified_base 8: mod_base = OTHER; note = Uracil
cgtgcgatatggtctcagcgggaagactttacgacgcaactaacatgaatgaata

SEQ ID NO: 25 - DNA, 1371 bp, Helicoverpa armigera
  source 1..1371: mol_type = other DNA
atggttgtcttgacctccaaagaaactaagccatctgttgctgaattttacgctggtaagtctgttttcattactggtggtactggtttcttgggtaagatcttcattgaaaagttgttgtactcctgcccagatatcggtaatatctacatgttgatcagagaaaagaagggtttgtccgtttccgaaagaatcaagcaatttttggatgaccctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcattcctcttacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtatgtttttacaaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcacctctcattcttgggttatgaaggctgatagagtcagagaattattcgcttctttgtctccagcagataagtacttgtttccatgtgatccaaccgatattaactggacccattacattcaagattactgctggggtgttagacacttcttggaaaaaaagactaccaacaagtaa

SEQ ID NO: 26 - DNA, 1362 bp, Heliothis subflexa
  source 1..1362: mol_type = other DNA
atggttgtcttgacctccaaagaaactaagccatctgttgctgaattttacgctggtaagtctgttttcattactggtggtactggtttcttgggtaaggttttcattgaaaagttgttgtactcctgcccagatatcggtaatatctacatgttgatcagagaaaagaagggtttgtccgtttccgaaagaatcaagcactttttggatgatcctttgttcaccagattgaaagaaaaaagaccagccgacttggaaaagatcgttttgattccaggtgatattactgctccagatttgggtattacctccgaaaacgaaaagatgttgatcgaaaaggtcagtgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagttcaccaatatgttaaggacggtatctctgaagaagaaactgaaaaaatcttgaacggtagaccaaacacttacactttcactaaggctttgaccgaacatttggttgctgaaaatcaagcttacgttccaaccattatcgttagaccatcagttgttgctgccattaaggatgaacctattaagggttggttgggtaattggtatggtgctacaggtttgactgtttttactgctaagggtttgaacagagttatctacggtcactcttctaacatcgttgatttgatcccagttgattacgttgccaacttggttattgctgctggtgctaaatcttctaagtctactgaattgaaggtctacaactgctgttcttctgcttgtaacccaattactatcggtaagttgatgtccatgtttgctgaagatgctatcaagcaaaagtcttacgctatgccattgccaggttggtacatttttactaagtacaagtggttggtcttgttgttgaccattttgttccaagttattccagcctacattaccgacttgtacagacatttgattggtaagaacccaagatatatcaagttgcaatccttggtcaatcaaaccagatcctccattgatttcttcaccaaccattcttgggttatgaaggctgatagagtcagagaattattcgcttctttgtctccagcagataagtacttgtttccatgtgatccagtcaacatcaattggagacaatatatccaagattactgctggggtgttagacatttcttggaaaaaaagacttaa

SEQ ID NO: 27 - DNA, 1362 bp, Heliothis virescens
  source 1..1362: mol_type = other DNA
atggttgtcttgacctccaaagaaactaagccatctgttgctgaattttacgctggtaagtctgttttcattactggtggtactggtttcttgggtaaggttttcattgaaaagttgttgtactcctgcccagatatcgttaacatctacatgttgatcagagaaaagaagggtttgtccgtttccgaaagaatcaagcaatttttggatgaccctttgttcaccagattgaaggacaaaagaccagctgatttggaaaagatcgttttgattccaggtgatattaccgctccagatttgggtattactgctgctaacgaaaagatgttgatcgaaaaggtttccgtcattattcattctgctgctaccgttaagttcaacgaaccattgccaactgcttggaagattaacgttgaaggtactagaatgatgttggccttgtctagaagaatgaagagaatcgaagttttcatccatatctctaccgcttacactaacaccaacagagaagttgttgacgaaatcttgtatccagctccagctgatattgatcaagtttaccaatacgtcaaagaaggtatctccgaagaagataccgaaaaaatcttgaacggtagaccaaacacttacactttcactaaggctttgaccgaacatttggttgctgaaaatcaagcttacgttccaaccattatcgttagaccatcagttgttgctgccattaaggatgaaccattgaaaggttggttgggtaattggtttggtgctacaggtttgactgtttttactgctaagggtttgaacagagttatctacggtcattccaactacatcgttgatttgatcccagttgattacgttgccaacttggttattgctgctggtgctaaatctaacacctcttctgaattgaaggtctacaactgttgttcctcatcatgtaacccagttaagatcggtactttgatgtctatgtttgctgatgatgccatcaagcaaaagtcttatgctatgccattgccaggttggtacatttttactaagtacaagtggttggtcttgttgttgaccttcttgttccaagttattccagcctacattaccgatttgtcaagacacttggttggtaagagtccaagatatatcaagttgcaatccttggtcaatcaaaccagatcctccattgatttcttcaccaatcattcttgggttatgaaggccgatagagtcagagaattatacgcttctttgtctccagcagataagtacttgtttccatgtgatccagttaacatcaactggacccaatacttgcaagattactgttggggtgttagaaacttcttggaaaaaaagacttaa

SEQ ID NO: 28 - skipped

SEQ ID NO: 29 - DNA, 1350 bp, Yponomeuta rorellus
  source 1..1350: mol_type = other DNA
atggtccaattgaaagaagattccgttgctgctttttacgccgaaaagtctattttcattactggtggtactggtttcttgggtaaggttttgattgaaaagttgttgtactcctgcaaggccgttgatcaaatctacgttttgatcagaaagaagaaggaccaaaccccatctgaaagaattgcccaattattggaatccgaattattctccagattgagaaaggatgatccatccgctttgaaaaaggttgttccagttgttggtgatttgaccatgcctaatttgggtttgtctgctgctgttgaagatttgattgtctctaaggttaccgttatcttccatgttgctgctactgttaagttcaacgaaagaatgaagaacgccttggttaacaacgttgaagctactagagaagtcatcaacttgtgccatagattggaaaaggttgatgccttcattcatgtttctaccgcttactctaataccgatcaaaaggtcgttgaagaaagagtttatccaccaccagctccattgtctgaagtttatgcttttgttaagaactacggtgacgacatggatatcatccaaaatttgttgaacggtagaccaaacacttacacctacacaaaagctttggctgaagatatcgtcttgaaagaacatggtggtattccaaccgctattatcagaccatctatcgttttgtccgttttaaaagaacctatcccaggttggttggataattggaatggtccaactggtttgttgcatgcttcttcacaaggtgttcattgctctatgttgggttctggttctaatgttgccgatttgattccagttgatatcgtcactaacttgatgatcgttgttgcttctagatgcagaaagtctaatggtttgaaggtctacaactcttgttctggtactaccaacccaattacttaccaagctttcaccaagatgttcttggattcctgtatttctagaggttggaacaaggttccattccctttgttgatttttgtcaagtgggctttcttgaacagagtcttgaagttcttgttggttatcgtcccattcttcttgatcgatgtctacttgagatttttcggtaagccaaactacatgagaatgatcacctacaccaagaaagctgaagatttgatgaccttcttcacctctcatgaatggcaattcaaggatggtaatgtcagagacttgatcaacatgatgtctccagaagatagaaagatcttctactgcgatcctgaagaaattcaatggaagccatacttcgatgattactgcgttggtgtctttaaatacttgttgaagagaaaggtttaa

SEQ ID NO: 30 - DNA, 1389 bp, Ostrinia nubilalis (E-strain)
  source 1..1389: mol_type = other DNA
atgtctgctaacactatggaaaccgatgaacaattcacttacaactccccaatcgtcaatttctactctggtaagtctgttttcgttactggtgctactggttttttgggtactgttttggttgaaaagttgttgttctcttgcaagggtatcaacaacatctacattttgatcaagcaaaccgaagatttgaccatcgaagccagaattttgaactacttgaactccaaggctttccacagagttaagaacactaatccagaattgatgaagaaaatcatcccaatctgcggtaacttggaagataagaacttgggtatctccgattctgatatgaagaccttgttggaagaagtctccatcgtttttcatgttgctgccaagttgttattcaagatgtctttgactgctgccgttaacattaacactaagccaaccgaacaattgattgccatctgtaagaagatgagaagaaacccaatcttcatctacgtttcttccgcttactctaacgtcaacgaacaaattattgacgaaaaggtctacaacactggtgttccattggaaactatctacgatactttggataccgaaaacaccagaattaccgatatcttcttggacaaaagaccaaacacttacacctactctaaagctttggctgaagttgtcgtcgaaaaagaatttgatgaatccgctgctatcgtcagaccatctattatcgtttcttctatcagagaacctatcccaggttggttgtctggttctcatggttttccaagagttgttggtgctgcttgtaaaggtttgttgttgagatggcatggtgatggtactgttgtttgtgatttgatcccagttgatcatgttgccaacttgattattgctgctgcttgggaatctaacgaaagaagattgatgggtaacaagggtgtcaaggtttacaactgttgttcctcattgagaaatccaatcgatgttatcaccgttgttaagacctgcatcaagtacagaaagtacttcggtactagaaccatgtctattttcaccccaagattcatcatgaagaagaactactttatctacaagttattatacttcacctaccacaccattccagccgctattattgatggttttttctggttgactggtagaaccccaattatgttgaaaaccttggataagttgggtaagatctcctccgttttggaatacttcactcatcaccaattcattttcttggactccaacgtcagaggtttgttaagaagaatggaaggtactgacagacaaaccttcaacttcgatgttaccgaaattgaatgggaaccatacttgcaaaacttcgttagaggtattgccaacaactacgactactctatgtga

SEQ ID NO: 31 - DNA, 1377 bp, Agrotis segetum
  source 1..1377: mol_type = other DNA
atgcccgtgctgacctcgcgagaggacgagaagctgtctgtgcccgagttctacgccggcaagtctatcttcgtgaccggcggcaccggattcctcggcaaggtgttcattgagaagctgctctactgctgccccgacatcgacaagatctacatgctgatccgagagaagaagaacctgtctatcgacgagcgaatgtctaagttcctggacgaccctctgttctctcgactgaaggaagaacgacccggcgacctcgagaagatcgtgctgatccccggcgacatcaccgctcctaacctgggcctgtctgccgagaacgaacgaatcctgctcgagaaggtgtccgtgatcatcaactctgccgccaccgtgaagttcaacgagcccctgcctatcgcctggaagatcaacgtcgagggcacccgaatgctgctggccctgtctcgacgaatgaagcgaatcgaggtgtttatccacatctctaccgcctactctaacgcctcttctgaccgaatcgtggtggacgagattctgtaccccgctcctgccgacatggaccaggtgtaccagctcgtgaaggacggcgtgaccgaggaagagactgagcgactgctgaacggactgcccaacacctacaccttcaccaaggctctgaccgagcacctggtggccgagcaccagacctacgtgcccaccatcatcattcgaccctccgtggtggcctctatcaaggacgagcccatccgaggctggctgtgcaactggttcggcgccaccggcatctctgtgttcaccgccaagggcctgaaccgagtgctgctcggaaaggcctctaacatcgtggacgtgatccccgtggactacgtggccaacctggtgatcgtggctggcgccaagtctggcggccagaagtctgacgagctgaagatctataactgctgttcttctgactgcaaccccgtgactctgaagaagatcatcaaggaattcaccgaggacaccatcaagaacaagtctcacatcatgcctctgcctggctggttcgtgttcaccaagtacaagtggctgctgaccctcctgaccatcatcttccagatgctgcccatgtacctggccgacgtgtaccgagtcctgaccggcaagattccccggtacatgaagctgcaccacctggtcattcagacccgactgggaatcgacttcttcacctctcactcttgggtgatgaagaccgaccgagtgcgagagctgttcggctctctgtctctggccgagaagcacatgttcccttgcgacccctcttccatcgactggaccgactacctgcagtcttactgctacggcgtgcgacgattcctggaaaagaagaagtag

SEQ ID NO: 32 - DNA, 1009 bp, Marinobacter algicola
  source 1..1009: mol_type = other DNA
atgtgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatggtgagtttcagaggcagcagcaattgccacgggctttgagcacacggccgggtgtggtcccattcccatcgacacaagacgccacgtcatccgaccagcactttttgcagtactaaccgcaggccacccagcagcagcagaacggtgcatccgcttcgggcgttctggagcagcttagaggcaagcatgtcttgattaccggtactacaggatttctgggaaaggtggttctggagaagctgatccgaaccgtgcctgacatcggtggtattcatctgctgattagaggcaacaagagacatcctgctgccagagaaagattcttgaacgaaatcgcctcttcctctgtgttcgagcggcttagacatgacgacaacgaagcctttgagactttcctggaggagcgtgtgcactgcatcaccggagaagtgaccgagtcgagatttggccttactcctgagcggttccgagcccttgctggccaagtggatgccttcatcaattccgccgcctctgttaacttcagagaggagctggacaaggcactcaagatcaacaccctgtgtctggagaacgtggctgctctggccgaacttaactccgctatggcagtgatccaagtttccacctgttacgtgaacggcaagaactctggacagatcaccgagtccgttatcaagcccgctggcgaatccatccccagatccacagatggctactacgagatcgaggagctggtccaccttctgcaagacaagatc

SEQ ID NO: 33 - DNA, 1407 bp, Chilo suppressalis
  source 1..1407: mol_type = other DNA
atggaaccctacaagtctgacgaggaaatcaagtctaagctgttctctcccgtggtgaacttctacaccggcaagtctgtgttcattaccggcgctaccggcttcctgggcaccgtgctgctcgagaagctgatgttcacctgtgctcacaacatcaagaacatctacatcctgatcaagcccaccgacggccagtctatcgacgagaagatgtctaagttcttcgactctcgagccttcgagcgactgcgagagcacaaccccaacttccgatctaagatcattcccctgaccggcgacatcaccaagaagtctatcggcctgtctgagaacgacatcttcattctgcgaaaggaagtgtctgtcgttttccactctgccgccgacacctctttccagctgtctctgtctgaggccatcatcatcaacaccaaggccaccgaggaactgctgaagatctgcaaggacatgcaccagctgaaggctttcgtgtacgtgtctaccgcctactctaactgcaaccgacctatcattgacgagaaggtgtaccctaccgacgtgtctctcgagactgtgtacgagctgctggaatactctaagtctgacaagctgatcgagttcctgttcaacggacgacccaacgcctacacctactccaaggctctgtcggaggaactggtgcagaactacggcaacatcatcccctctatcatcattcgaccctccatcatcacctcttctatcaaggaaccctatcctggctggctgtctggctggaacggcctgaaccaggtgatcctgtctggcatgaagggctacctccgatgctggttcgccgacgactcttgtatcgccgacactatccccgtggactacaccgccaacgtgatgatcgtgtctgcctgggacgcccacgagcgacgactgaagaacgacaagcagctcaaggtgttcaactgctgctctggcctgcagaaccccatcgacaccggaaacatgatgtctatctgcctcgagcacaacaagaagcacgagaaggacaagtccaaggccaacaccatgttcatcatccgaaagaacttttacgtgtacttcttctacttcctggtgctgcatctgatccccgctctgattatcgacgtgttctactttctgctgggccgagagatgctgatgtgtaccaacctgaagaagatcaagcgattctcttcgatcctgaaggtgttctgcttcaaccagtttctgttcatcgacaagaacgtccgacgactgcaccaggctctgaacgagactgacaaggtgctgttcgacttcgacgtgcgaaacatccagtggcgagtgtacctgaaggacttcgtgatcgccctgaaggaatacgacaagacctctcgaaccgtgaagatttag

SEQ ID NO: 34 - DNA, 1365 bp, Spodoptera littoralis
  source 1..1365: mol_type = other DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggacaagcgacccggcgacctgggaaagatcgtgctgatccccggcgacatcaccgtgcctggcctgggcatctctgaggaaaacgagactatcctgaccgagaaggtgtctgtggtgatccactctgccgccaccgtgaagttcaacgagcccctggccaccgcctggaacgtgaacgtcgagggcacccgaatgatcatggccctgtctcgacgaatgaagcgaatcgaggtgtttatccacatctctaccgcctacaccaacaccaaccgagccgtgatcgacgaggtgctgtaccctcctcctgccgacatcaacgacgtgcaccagcacgtgaagaacggcgtgaccgaggaagagactgagaagatcctgaacggacgacccaacacctacaccttcaccaaggctctgaccgagcacctggtggccgagaaccagtcttacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccactctaaccacgtcgtggatctgattcccgtggactacgtggccaacctggtgatcgtggctggcgccaagacctaccactccaacgaggtgaccatctacaactcttgttcttcttcttgcaaccccatcaccatgaagcgactggtgggcctgttcattgactacaccgtcaagcacaagtcttacgtgatgcccctgcctggctggtacgtgtactctaactacaagtggctggtgttcctggtgaccgtgatcttccaggtgatccccgcctacctgggcgacatcggccgacgactgctgggcaagaaccctcggtactacaagctgcagaacctggtcgctcagacccaagaggccgtgcacttcttcacctctcacacctgggagattaagtctaagcgaacctctgagctgttctcttctctgtccctgaccgaccagcgaatgttcccctgcgacgccaaccgaatcgactggaccgactacatcaccgactactgctctggcgtgcgacagttcctcgagaagatcaagtag

SEQ ID NO: 35 - DNA, 1365 bp, Spodoptera exigua
  source 1..1365: mol_type = other DNA
atggtggtgctgacctctaaggaaaagtctaacatgtctgtggccgacttctacgccggcaagtctgtgttcattaccggcggcaccggattcctcggcaaggtgttcatcgagaagctgctgtactcttgccccgacatcgacaagatctacatgctgatccgagagaagaagggccagtctatccgagagcgactgaccaagatcgtggacgaccctctgttcaaccgactgaaggaaaagcgacccggcgacctggacaagattgtgctgatccccggcgacgtgaccgtgcctggcctgggcatctctgacgagaacgaggccatcctgatcgacaaggtgtctgtggtgatccactctgccgccaccgtgaagttcaacgagcccctcgagactgcctggaacgtgaacgtcgagggcacccgaatgatcatggccctgtctcgaaagatgaagcgaatcgagatcttcatccacatctctaccgcctacaccaacaccaaccgagccgtggtggacgaggtgctgtaccctcctcctgccgacatcaacgaggtgcaccagtacgtgaagaacggcatcaccgaggaagagactgagaagatcctgaacggacgacccaacacctacaccttcaccaaggctctgaccgagcacctggtggccgagaaccaggcctacatgcccaccatcatcgtgcgaccctctatcgtgggcgccatcaaggacgaccccatccgaggctggctggccaactggtacggcgccaccggcctgtccgtgttcaccgccaagggcctgaaccgagtgatctacggccagtcctctcacgtggtggatctgattcccgtggactacgtggccaacctggtgatcgtggctggcgccaagacctaccgatctaacgaggtgactatctacaactcttgttcttcttcttgcaaccccatcaccatggaacgactggtgggcctgttcattgacgacaccgtcaagcacaactcttacgtgatgcccctgcctggctggtacgtgtactctaactaccgatggctggtgtacctggtgaccatcatcttccagatgatccccgcctacctggccgacatcggccgacgactgctgggcaagaaccctcggtactacaagctgcagtctctggtggctcagacccaagaggccgtgcacttcttcacctctcacacctgggagatcaagtctaagcgaacctctgagctgttcgcctctctgtctcataccgaccagcgaatcttcccatgcgacgccaagaagatcgactggaccgactacatcaccgactactgctctggcgtgcgacagttcctggaaaagaagaagtag

SEQ ID NO: 36 - DNA, 1380 bp, Spodoptera exigua
  source 1..1380: mol_type = other DNA
atgacctaccgacagatcaacgagttcgacgccgagaagttcaccgccgctgctgtgcccacctcttacgtgtctgtgcccgacttctacgccggcaagtctatcttcattaccggcggcaccggattcctcggcaaggtgttcctcgagaagctgctgtactcttgcaaggacgtcgagactgtgtacatcctgatccgagagaagaagggcaagacccctcagcagcgagtcgaggacctgttcaacaagcccatcttctcgcgactgaagcaaaaggactctcagtgcatgaagaaggtgaccgccatcatcggcgacctgtctgagcccggcctgggcatctctaaggacgacgaggaactgctgctgcaaaaggtgtctgtggtgttccacgtggccgccaacgtgcagttctacaaggaattcaaggaaatcatcaacaccaacgtcggaggcaccaagtacgtgctgcagctgtgccagcgaatcaaggacatcaaggccttcgtgcacatctctaccgcctactgccacaccgaccagaaggtgctggaagaacgaatctaccctcctcctgccgaactgtctgaggtgctgaagttcctgcagcagccccagcacgacaagaagcagatcaaggaactgtttaagaagcagcccaactcttacaccttcgccaaggctctggccgagacttacattgccgagaactgcggacgagtgcccaccatcatcattcgaccctctattatctctgcctctctgaaggaacccctgcctggctgggtcgactcttggaacggcgccaccggcctgatcaccgcctcttacaacggcgctaaccgagtgctgctcggcgaaggcggcaacttcctggacctgattcctgtggacttcgtggccaacctggccatcgtggccgctgccaagtgcacctcttcgctgaaggtgtacaactgctgttcttctggctgcaaccctctgactctgaagcagctggtgtctcacatgaacaacgtgggcttcgacaagaacgtgtctatcatctttaccaacaacaaggcctctctgtctaccctgacattcttcctgcagaccactccttcttttaccgccgacatgttcctgcgagtgaccggaaagtctccccgatatatgaagatccagtctaagctgaccattgctcgaaacgccctgaacttcttcacctgtcactcttgggtgatgaaggccgacaactctcgacgactgtacgcttctctgtctctgcacgaccgacacactttcccttgcgaccccaccgacatcgactggaagaagtacatcaccatctacatcgagggaatcaaccagttcctgatgaagaagcgatcttag

SEQ ID NO: 37 - DNA, 1353 bp, Spodoptera frugiperda
  source 1..1353: mol_type = other DNA
atgtctaaggccaactgctctaacgtggcctcgttctacgccggcaagtctgtgttcatcaccggcggcaccggcttcgtgggcaagaccctgatcgagaagctgctgtactcttgctctggcatcgacaagatctacgtgctggtgcgagacaagtacggcaagcaggcctctgagcgactggcccgagtgacctctactcccgtgttcgaccgactcaagacctctaagtctgaggaactgaagaagatcgtcgtcatctctggcgacatcacccgagagaacctgggcctgcaagaggacgtgctcaagaccctcgagaacgaggtgtctgtggtgttccacctggccgccaccgtggccttcaagctgcccctgaaggacgccatgcgaatcaacgtgaacggcaccgagaacgtgatcgagctgtgccgacgaatgaagaagctgcaggccttcgtccacgtgtctaccgccttcaccaactctgaccgaaaggaaatcaacgagatggtgtaccccatgcctatcaacctggaagaggcccgacacgtggcctacacctactctcacgacgacgtgatcgtgtctgccttcatgggaaagaagcccaacacctacaccttctccaaggccttcgccgagcagcaggtcatgaagcagtgcgaggacctgcctaccgccatcattcgaccctctatcgtgatgtcctctgtgaaggaaccctgtcctggctggatcgacacctggaacggctctaccggcctgttcgtgggaatgcccgctggcatcctgcgagtggtgctcggacgaggatctaacgtgaccgacctggtgcctgtggacatcgtgaccaacctgatcgtggtggccgctaccgagtgccagaagtctaaggaaatcaaggtctacaactgctgcaccggcacttctaaccccatcacctgtgacgaggctaccaccatctctcgaaaggtggccctgaagtactctctgaacgagctgccctggcctttcctgatgtttaccccttctgtgttcctgtataccctgatcaccttcctgctgcagatcgtgcccgcctacctgatcgacctgtggtgcatcctgaccggccgaaaggctacccagatcaagctgcagcgacgacttaagaaggtggtcgacgccgtcaagttctttctgctgaacgagtggaagttctctgacgacaacattcgagatctgcttaagaccatgtctcccgtggacaaggaaatcttcaacttcgacgtcaagaccatcaactgggagacttgcatcgaggactacatcctgggagcccgaaagtacctgctgaagtctgagaagtag

SEQ ID NO: 38 - DNA, 1407 bp, Plutella xylostella
  source 1..1407: mol_type = other DNA
atgaagctgggcaccttcggcaacatggccgccaacggcggctctgtgtgcgacttctacgccaactctaccgtgttcattaccggcggcaccggattcctcggcaaggtgctgatcgagaagctgctgcgatcttgccgaaagatcgacaagatctacgtcctgctgcgaggcaccaccaacaagaccgctcaagagcgactcgaggacctgtctcacgaggtcctgttcgagaaggtgcgacaggacgaccccaacatcatgaagaagctgatccccgtcaacggcgacctgactctgcccaacctgggcatgaacaacgaggactactctctgatcgtgaagaacgtgtctatcgtgttccacgccgctgcctctatccgattcaacctgaagatcaaggaagccctgcactgcaacgtcgagggcaccaagaaggtgatcgagctttgccacaaggcccgatgcatcaaggccttcgtgcacatctctaccgcctactctaacaccgacaagaccttcgtgaaggaagaggtgtaccccgctcctgagaacctggataagatctatgtgatcatcggccagaacggcgacgacaccgaggccgacgacctgacctcttctcgacccaacacctacacctacgccaaggccgtggccgagtacgaggtgctgcgaaaccacggatctgtgcccaccgccatcattcgaccctctatcgtgacccctatctggcgagatcccactcctggctggctggacaagtggtctggcggcacccctatcatcttcggcctgtgcaagggcctgctccgatgcctggacacccagccttctaacgtgatcgacttcatccccgccgacattgccgtgaacctgtccatcgtggccgctgccaagtgccacgtggacacccctaagatccgagtgtacaactgctgctctggccctacaaaccccatgaccttcaagatgctgtctgactactgccacgagaactctatcaagcacggcctgcactggttcccttatcctaagatcttcctgtctaccaaccgagtgttcctgaagattgtgcgactggtgctgcagttcctgcctctgttcctggccgacatgtggctgaagattcgaggcaagcagccccgattcgtgaagatgctggaaaagctgaccaaggtggaaaagaccctgcacttcttcatcaagaaccagtggcagttcgagaaccacaacgttgaggccctgatctctaccctgtctccttcggaccgagtgcagttcccttgcgacccctctgagatctgctggaagacccacttcgaggattactgcctgggcatcgacaagtacctgatgaacacctctgtgaagtctgccaacatcaactctggcatgtacaagtag

SEQ ID NO: 39 - DNA, 1608 bp, Plodia interpunctella
  source 1..1608: mol_type = other DNA
atgcgactggtgccctctgctctggacgtggtgctggccgccgagcgacgactggtccccatccacgaggctaccctgaagggcgactctgaggtgcagaagttctacgccggctctgtggtcctggtgaccggcggctctggcttcctgggcaagcagctgatcgagaagctgttccgaacctgcgacgtgaagaagctgtacatcctgctgcgagccaagaagggcaagtctatcggcgagcgactggactacattctgcgaaaccccgtgtacgacaccctgcgagagactcgaccctggttcgccgaccgaatcgaggccgtcgagggcgacattgcctcttaccgactgggcgtcgccgaccatatgtgggaccgactggtggacgaggtgaccgtggtgttccacggcgctgccaccatcaacttcgctgaccctctgcagctggccaccaacatcaacgtgaagggcaccctcgagatgctcgagttcggcaaggcctgcaagaacctccgatcttacgtgcacatctctaccgcctacaaccaggctctgaagaacaacgtgtcttgcgacatcctggaagagttccccaaggctcccctgtctcctaccactctggtggacctggccaaccgaaccgagactaaggtgctggacgagatgttccacaagtacctggcacctgagtaccccaacacctacgcctacaccaaggccgtggccgaagaggccgtgcgaaccatggccggcgacctgcctatctgcgtggtgcgaccctctatcgtgatccccgcctacactgagccctctgccggctgggtcgacaagaactctatctacggcgcctctggcatcatcatgggcctcggcctgggcgtgattcacaccctgatctctgaccccgacatcaagatcgacatcatccccgtggacatcgtgaacaacgccgtgatcgtggccggatgggagactgcccgacgacgagagcagggcgagactgacaccaagatctacaccgtgggatcttctaaccgaaaccacatcacctggcgatttctgaccaacgtgttcgacaacgagggccgaaagcacctgtcctctcaggccgtgtggtacgccttcgccgtgcagaccaagtctaagcacctctacctggctctggcctggctgctgcacttcatccccggctacatcgtggacggcgtgctgatgatccgaggcaagaagccccaggtttctaagatctaccgactgctgaacactatggccaccatcttctcgttcttcaccctccgaggctggaacttcaaggacgacaacctgctgcagctgtacaagtctctgtctaagaccgaccagcagatcttcaacatggacattgctaccctggacatgaaggaagtgatctacctctggcacattggcctgcgacgattctacctgaaggacaagctgaacgaccaagagaagtctatgaagaagcagttcttcctgcgaatcatcacctacaccttcattcctatctacctgtacttcctgttcaagatctcttgcttcgtgttctacggcctgtactacctgatcttctcttacctgatttga

SEQ ID NO: 40 - DNA, 1632 bp, Amyelois transitella
  source 1..1632: mol_type = other DNA
atggccgccaagtgcgagatcggcgtgtctcagttcttcgccggcaagcacgtgttcatcaccggcgccaccggcttcatgggcaaggtgctggtcgagcgactgctgtctacctgcgccgacgtgggccgaatctacctgctgatgcgatctaagaaggaccactctcctgagcagcgactgcaagagctgaaggcctctcaggccttcgacaacctgcgacagtctcagccctctcagctcgagaagctgcgaatcgtggccggcgacatcatgctgcccggcctgggcatctctcaagagggcctcgaggacctgcaagaggtgtctgtggtgttccactctgccgccaccgtgaagttcaacgaggccctgaagatcgccatcgagcagaacctgatgtctgtgatccgactgctcgagatctgcgaccgactgcctcacatgcaggctttcgtgcacgtgtctaccgcctactctaaccccgagctgtccaccgtcgaggaacgagtgtaccctcctcctgtgcctctggactacctgctcgacctggccgccgaggactctcccctgctggacaacgtcgagtctttcatctctcccaagcctaactcttacaccttcaccaaggccatggctgaggccgccgtgcagcgacacggctgccagtactaccccattgccatcgtgcgacccaccatcgtgatctcttcgctgaagcacccctttccaggctggctcgagaacgccaacggacccaccggcgtgattttcggagccggccgaggcctgctgcgagtgttccgatgccgacagtctgccctggccgacctgctgcctgtggacatcggcatcgacaccctgatcgccgctgcctgggaagtcgcctctgaccgacctccttcgcctcgagtgtacaactgctctatctctgagaaccccaccacctggggcgacctgaagcgagccatcaacctgcacacccgatctcagccccagtgtggcgccgtgtggtatcccggactgcataccgccgagaacggcttcctgtacaagatcctcgaggtgctgctgcagaccactcctctgtacatcggcgagatcctggtgcgaatcctgggctactctcacaagcgactgaacctgatccgagtgtctacccagatgaagaacatgcagaacgccatgaagtactttgccctgcgagagtggcgattccagtctgacaacgtgcgacgactgcgagagcgaatgtctgacgtggacaagcgaatctacaacctggatccttgctctatcgtgtgggacgagcactactccaactacgtgaagggcgtgcgactgtacctcctccgagagaaggaactggaccagcctgccgccaagaagcgactcaagaagctctactacctgcactacggcctgtctgtgttcatgccctttctgctgttccgagccttcgtccgacgaaagcaccgacagaacattctcgaggtcctgttcgccatcctgtctatcttcaacgccatcttccgacgatcgacccagaacatcctgtacatctacaaggtgtctctgaccttcctgaacgacaacgtgttcctgaacaaggtgtttaacgagtcctcttag

SEQ ID NO: 41 - DNA, 1389 bp, Ostrinia zea
  source 1..1389: mol_type = other DNA
atggccgccaacaccatcgaaaccgacgagcagttcaccgacaactctcccatcgtgaacttctactctggcaagtctgtgttcgtgaccggcgctaccggcttcgtgggcaccgtgctggtcgagaagctgctgttctcttgcaagggcatcaacaacatctacatcctgatcaagcagaccgaggacctgaccatcgaggcccgaatcctgaactacctgaactctaagcccttccaccgagtgaagaacactaaccccgagctgctgaagaagatcatccccatctgcggcaacctcgaggacaagaacctgggcatctctgactctgacatgaagaccctgctggaagaggtgtctatcgtgttccacgtggccgccaagctgctcttcaagatgaacctgaccgccgctgtgaacatcaacaccaagcctaccgagcagctgatcgccatctgcaagaagatgcgacgaaaccccatcttcatctacgtgtcctctgcctactctaacgtgaacgagcagatcatcgacgagaaggtgtactctacccgagtgcccctcgagactgtgtacgacaccctggacatcgagaacacccgaattaccgacatcttcctggacaagcgacccaacacctacacctactccaaggctctggccgaggtggtcgtcgagaaggaattcaacgagtctgccgccatcgtgcgaccctctatcattctgtcttctattcgagagcccattcctggctggctgtctggctctcacggcttccctcgagtggtcggagccgcctgcaagggcctgctgctccgatggcacggcgacggcaccgtggtgtgcgacgtgatccccgtggaccacgtcgccaacctgtctattgccgtggcctgggagtctaacgagcgacgactcatgggcaacaagggcgtgaaggtctacaactgctgctcttctctgcgaaaccctatcgacgtgtctaccgtgctgaacacctgtctgaagtaccgaaagtacttcggcacccgaaccatgtctatcttcacccctcgattcattatgaagaagaactacttcctgtacaagctgctgtacttcacctaccacactatccccgctgctattatcgacggcttcttctggctgaccggacgaacccctatcatgctcaagaccctggacaagctccgaaagctgtcctctgtgctcgagtactttacccaccaccagtttctgtttctggactccaacgtgcgaggactgctgcgacgaatggaaggcaccgaccgacagaccttcaacttcgacgtcaccgagatcgagtgggagccctacctgcagaacttcgtgcgaggcattgccaacaactacgacaactctatgtag

SEQ ID NO: 42 - DNA, 1362 bp, Trichoplusia ni
  source 1..1362: mol_type = other DNA
atgatcgccgtgacctctcacgagaacgaggcctctatcgtggacttctacgagggcaagtctgtgttcattaccggcggcaccggattcctcggcaaggtgttcatcgagaagctgctgtactgctgtcccggcctggtgaagatctacatgctgatccgagagaagaagggcctgaccatcaaggaacgaattgaccgattcgtggacgactctctgttcgaccgactcaagacccagcgacctaaggacctggacaagatcgtgctgatccccggcgacatcactgccccttctctgggcatctctcaagagcaagagaacatcctgatcgaagaggtgtctgtgatcatccactctgccgccaccatcaagttcaacgcccctctggaagaggcctggtctatcaacgtggacggcacccgacgaatcctggacctgtgccgaaacatgaagaaggtcgaggttttcctgcacatctctaccgcctacaccaacatcaaccgaaacgtgatcgaggaagtgctctaccctcctccagccgacattaaggaactggaaaagctggacaagcacaacgtgaccgagaagcagaccatgaagctgctcaacggcttccccaacacctacaccttcaccaagtctctgaccgagcacctggtggccgagaactgcgcctacatgcccaccatcatcgtgcgaccctctgtggtggccgccgtgctcgaggaccccatcaagggctggctggaaaactggtacggcgccaccggcatctccgtgttcaccgccaagggactgaaccgagtgttcctgggccacaagaacattattgtggacgtgattcccgccgactacgtggccaacctggtgatcattgccggcgctcgaaacaacaagtccaaggaactcaagatctataactgctgttcttctggctgcaaccctctgaccatcggcaacctgctgggcgccttcatcgacgacgccatccgacacaagacctacgctatgcccctgcctggctggttcatcttcacccgataccgactggtggctaccatcgtgaccttcatcttccaggtggtgcccgcctacctggccgacctgtaccgacgaattatcggcaagaagcccaagtacaccaagctgctctacctggtggtgcagacccgagtggccctggacttcttctcctctaactcttgggtgatgaaggccgaccgaatgcgagagctgtacgcctctctgtctaacaacgacaagcgaatgttcccttgcgaccccactcacatcaactggcccgagtacctgtctgactacggcgctggcgtgcgacgattcctcgagaagcgaaactag

SEQ ID NO: 43 - DNA, 1386 bp, Trichoplusia ni
  source 1..1386: mol_type = other DNA
atgttcacctccaccaacaacatccgaaccgaggacgacgtgaacgccgagcacgtcgagtctatcgccgacttctacgccggcaagtctgtgttcattaccggcgtgaccggcttcctcggcaaggtgtacctcgagaagctgctgtacacctgtaaggacatcgacaaggtgttcatcctgatccgaaacaagaagggcgacgacgtcggcaagcgaattgagaagctcctggactcttctgtgttctctcgactgaagtctcagcgaccccaggacatcaagaagatcgtccccgtctacggcgacatggacaagtctgagctgggcctgtcctctgaggaccaagagcgactgatcaacgaggtgtctgtggtgttccacgtggccgcctctgtgaacctggacgccgacctgcgatcttctatggctaccaacttcttcggcacctcttacgtgctgaagctgtgccaccgaatgaagaagatcaaggttttcgtctacgtgtctaccgcctactgcaactctaccctgaaggtgctggaagagaaggtctaccccgtgcaagaggaactggaagaggtcctgaagtacctggaagaaccccacctggaccgagatcgagtgaagaagctgttgaacggacgacccaacgcctacgctctgtctaaggccctggccgagcactacgtggcccagaaccacggtgacatccccatcattttcattcgaccctctatcgtgatctcttcgtctaaggaacccgtgcctggctgggtcgactctttccagggcgccaccgctctgatcaccgccgtcctgaagggcgtgaaccgagtgatctacggcaacaagaacaacgtgctggacctgattcctgtggactacgtgtccaacctgtctatcgtggccgctgctcgagtcaagtcctctaaggaagtgaccgtctacaactgctgcacctcttctatcaagcccatcactctgaaggaactggcctctcagatcacccgacgaaaggtcgaggacaagcagcgattcctgttcctgcctaccgtgttcttctctacccactggctggtggtgatcattctgaccttcgtgatgcagatcatgccctcgtttattgccgaccagttcctgtacttcaccggaaacaagcctatgtacatgaagatccagtctaaggtgatcaacggccgaaacgtcctgaactactacactaacaactcttgggtgttcaagtccgtggcctctcgacgactgctggacaccctgtctctgtctgacaagcgactgttcccttgctacccctctgacctgaactgggacaagtactttaagacctacttcgacggaatcgagaacttcctgctgaagaagtag

SEQ ID NO: 44 - DNA, 1353 bp, Chrysodeixis includens
  source 1..1353: mol_type = other DNA
atgtcctcgctgaagggccccaaggtggcctcgttctacaccggcaagtctgtgttcattaccggcggaaccggcttcctgggcaagaccctgatcgagaagctgctgtactcttgctctggcatcgacaagctgtacatcctggtgcgagacaagcgaggctacgccgctcacgagcgactggcccgaatcaccaacactcccgccttcgaccgactgaagggcacccgaaacgaggacctgaagaagatcaccgtcatcaccggcgacattgctcgacccaacctgggactgcccgacgacatcctgaagcgactcgaggacgaggtgtctgtggtgttccacctggccgccaccgtggccttcaacctgagactgaaggacgccatgggcatcaacatcaacggcaccgaagaggtggtgaagctgtgccaccgaatgaagaagctgcaggccttcgtccacgtgtctaccgccttctctaactctgaccgattcgagatcgaagagaaggtgtaccccatgcctgtgactcttgaggacgcccgacacgtggccgagatgtactctcacgacgacgaggtgattacccagttcctgggaaagaagcccaacacctacaccttttctaaggccctggctgaggacctggtgatgaagcagtgtcaggacctgcctgccgccatcgtgcgaccctctatcgtgatgtcctctatcaaggaaccctgtcctggctggatcgacacctggtctggctctaccggcctgttcgtgggaatgcccgctggcgtgctgaaggtggtcaagggcaagggctctaacgtgaccgacctggtgcctgtggatatcgtgaccaacttcctgatcgtggtcgccaccgagtgccaaaagaccaacgagatcaaggtgtacaactgctgcaccggcacttctaaccccatcacctctaactacgcctctgctatcgcccgacgagtgaccctgcgatactctctgaacgagctgccctggcctttcctggtgttcacctcttctctgattgtgtacaccctgattaccttcatgctgcagactatccccgccttcctgattgacctgtggtgcgtgctgaccggcaagaaggctacccagctgaagctccagtctcgactcaagaagaacatcgaggtcgtcaagttcttcctgctgaacgagtggaagttctgcgacgagaacgtgcgaaacctgttcaagtctctgtctcccgaggacaaggaaaccttcaacttcgacgtgaagtctatcaactgggagaagtccatcgaggactacattctgggagcccgaaagtacctgctgaagtacgagaagtag

SEQ ID NO: 45 - DNA, 1386 bp, Chrysodeixis includens
  source 1..1386: mol_type = other DNA
atgttcgcctctaccgacggcggacgatctgacgaggaaacccctcagcagcacgagtctatcgccgacttctacgccggcaagtctgtgttcattaccggcgtgaccggcttcctcggcaaggcctacctcgagaagctgctgtactcttgcaaggacatcgacaaggtgttcgtgctgatccgaaacaagcgaggcgacgacgtgggcaagcgaattgagaagctcctggactcctctgtgttctctcgactgcgatctcaccgacctgaggacctgaagaagatcacccctgtctacggcgacattgacaagtctgagctgggcctgtctctggccgaccaagagcgactgatcaaggaagtgtctgtcgttttccacgtggccgcctctgtgaacctggacgccgacctcaagacctctctggtgaccaactacttcggcaccactcacgtgctgaagctgtgccaccgaatgaagaagctgaaggttttcgtctacgtgtctaccgcctactgcaacaccaccgtgaaggtgctggaagagaaggtgtaccctctgcctgtcgagctggacgaggtggtgaagatcatggaacagccccacctggactctaaccgagtcaagaagttgctgaacggacgacccaacacctacgctctgtctaaggccctggccgagcactacattgccgagaaccacggtgacatccccgtgattttcattcgaccctctatcgtgacctcttctgctcgagagcccgctcctggctgggccgactctttccagggcgccaccgctctgatcaccgcctgctggaagcgagtgaaccgagtgatctacggcaacggcgacaacatcatcgatctgatccccgtggactacgtgtccaacctgtctatcgtggccgctgccaaggtgaagtctaccaccgagatcgccgtgtacaactcttgcacctcttctatcaagcccatgactctgaagggaatcgccgagcagatcgagcgagtcaacaccgagaacaagcagggctctatcttcctgcctactattttcttcacctcctcttggctgttcgtgatcatgctgaccttcgtgctgcagctgctcccctcttttattgccgacctgttcctgtacatcaccggcaacaagcctatgtacatgaagatccagtctaaggtgctgcacggccgacacatcctgaactactacaccaacgactcttgggtgttcaagtctgactcttctcagcgactgcaggactctctgtctcccgccgacaagcgactgttcccctgctgtccctctgacatcaagtgggacgagtactttaacacctacttttgcggcatcgacaactacctgctcaagcgaaagtag

SEQ ID NO: 46 - protein, 455 aa, Helicoverpa armigera
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHRYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKSYE

SEQ ID NO: 47 - protein, 453 aa, Heliothis subflexa
MVVLTSKETKPSVAEFYAGKSVFITGGTGFLGKVFIEKLLYSCPDIGNIYMLIREKKGLSVSERIKHFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSNIVDLIPVDYVANLVIAAGAKSSKSTELKVYNCCSSACNPITIGKLMSMFAEDAIKQKSYAMPLPGWYIFTKYKWLVLLLTILFQVIPAYITDLYRHLIGKNPRYIKLQSLVNQTRSSIDFFTNHSWVMKADRVRELFASLSPADKYLFPCDPVNINWRQYIQDYCWGVRHFLEKKT

SEQ ID NO: 48 - protein, 453 aa, Heliothis virescens
MVVLTSKETKPSVAEFYAGKSVFITGGTGFLGKVFIEKLLYSCPDIVNIYMLIREKKGLSVSERIKQFLDDPLFTRLKDKRPADLEKIVLIPGDITAPDLGITAANEKMLIEKVSVIIHSAATVKFNEPLPTAWKINVEGTRMMLALSRRMKRIEVFIHISTAYTNTNREVVDEILYPAPADIDQVYQYVKEGISEEDTEKILNGRPNTYTFTKALTEHLVAENQAYVPTIIVRPSVVAAIKDEPLKGWLGNWFGATGLTVFTAKGLNRVIYGHSNYIVDLIPVDYVANLVIAAGAKSNTSSELKVYNCCSSSCNPVKIGTLMSMFADDAIKQKSYAMPLPGWYIFTKYKWLVLLLTFLFQVIPAYITDLSRHLVGKSPRYIKLQSLVNQTRSSIDFFTNHSWVMKADRVRELYASLSPADKYLFPCDPVNINWTQYLQDYCWGVRNFLEKKT

SEQ ID NO: 49 - protein, 456 aa, Helicoverpa assulta
MVVLTSKETKPSVAEFYAGKSVFITGGTGFLGKIFIEKLLYSCPDIGNIYMLIREKKGLSVSERIKQFLDDPLFTRLKEKRPADLEKIVLIPGDITAPDLGITSENEKMLIEKVSVIIHSAATVKFNEPLPTAWKINVEGTRMMLALSRRMKRIEVFIHISTAYTNTNREVVDEILYPAPADIDQVHQYVKDGISEEETEKILNGRPNTYTFTKALTEHLVAENQAYVPTIIVRPSVVAAIKDEPIKGWLGNWYGATGLTVFTAKGLNRVIYGHSSYIVDLIPVDYVANLVIAAGAKSSKSTELKVYNCCSSACNPITIGKLMSMFAEDAIKQKSYAMPLPGWYVFTKYKWLVLLLTILFQVIPAYITDLYRHLIGKNPRYIKLQSLVNQTRSSIDFFTSHSWVMKADRVRELFASLSPADKYLFPCDPTDINWTHYIQDYCWGVRHFLEKKTTNK

SEQ ID NO: 50 - protein, 449 aa, Yponomeuta rorellus
MVQLKEDSVAAFYAEKSIFITGGTGFLGKVLIEKLLYSCKAVDQIYVLIRKKKDQTPSERIAQLLESELFSRLRKDDPSALKKVVPVVGDLTMPNLGLSAAVEDLIVSKVTVIFHVAATVKFNERMKNALVNNVEATREVINLCHRLEKVDAFIHVSTAYSNTDQKVVEERVYPPPAPLSEVYAFVKNYGDDMDIIQNLLNGRPNTYTYTKALAEDIVLKEHGGIPTAIIRPSIVLSVLKEPIPGWLDNWNGPTGLLHASSQGVHCSMLGSGSNVADLIPVDIVTNLMIVVASRCRKSNGLKVYNSCSGTTNPITYQAFTKMFLDSCISRGWNKVPFPLLIFVKWAFLNRVLKFLLVIVPFFLIDVYLRFFGKPNYMRMITYTKKAEDLMTFFTSHEWQFKDGNVRDLINMMSPEDRKIFYCDPEEIQWKPYFDDYCVGVFKYLLKRKV

SEQ ID NO: 51 - protein, 462 aa, Ostrinia nubilalis (E-strain)
MSANTMETDEQFTYNSPIVNFYSGKSVFVTGATGFLGTVLVEKLLFSCKGINNIYILIKQTEDLTIEARILNYLNSKAFHRVKNTNPELMKKIIPICGNLEDKNLGISDSDMKTLLEEVSIVFHVAAKLLFKMSLTAAVNINTKPTEQLIAICKKMRRNPIFIYVSSAYSNVNEQIIDEKVYNTGVPLETIYDTLDTENTRITDIFLDKRPNTYTYSKALAEVVVEKEFDESAAIVRPSIIVSSIREPIPGWLSGSHGFPRVVGAACKGLLLRWHGDGTVVCDLIPVDHVANLIIAAAWESNERRLMGNKGVKVYNCCSSLRNPIDVITVVKTCIKYRKYFGTRTMSIFTPRFIMKKNYFIYKLLYFTYHTIPAAIIDGFFWLTGRTPIMLKTLDKLGKISSVLEYFTHHQFIFLDSNVRGLLRRMEGTDRQTFNFDVTEIEWEPYLQNFVRGIANNYDYSM

SEQ ID NO: 52 - protein, 458 aa, Agrotis segetum
MPVLTSREDEKLSVPEFYAGKSIFVTGGTGFLGKVFIEKLLYCCPDIDKIYMLIREKKNLSIDERMSKFLDDPLFSRLKEERPGDLEKIVLIPGDITAPNLGLSAENERILLEKVSVIINSAATVKFNEPLPIAWKINVEGTRMLLALSRRMKRIEVFIHISTAYSNASSDRIVVDEILYPAPADMDQVYQLVKDGVTEEETERLLNGLPNTYTFTKALTEHLVAEHQTYVPTIIIRPSVVASIKDEPIRGWLCNWFGATGISVFTAKGLNRVLLGKASNIVDVIPVDYVANLVIVAGAKSGGQKSDELKIYNCCSSDCNPVTLKKIIKEFTEDTIKNKSHIMPLPGWFVFTKYKWLLTLLTIIFQMLPMYLADVYRVLTGKIPRYMKLHHLVIQTRLGIDFFTSHSWVMKTDRVRELFGSLSLAEKHMFPCDPSSIDWTDYLQSYCYGVRRFLEKKK

SEQ ID NO: 53 - protein, 512 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 54 - protein, 468 aa, Chilo suppressalis
MEPYKSDEEIKSKLFSPVVNFYTGKSVFITGATGFLGTVLLEKLMFTCAHNIKNIYILIKPTDGQSIDEKMSKFFDSRAFERLREHNPNFRSKIIPLTGDITKKSIGLSENDIFILRKEVSVVFHSAADTSFQLSLSEAIIINTKATEELLKICKDMHQLKAFVYVSTAYSNCNRPIIDEKVYPTDVSLETVYELLEYSKSDKLIEFLFNGRPNAYTYSKALSEELVQNYGNIIPSIIIRPSIITSSIKEPYPGWLSGWNGLNQVILSGMKGYLRCWFADDSCIADTIPVDYTANVMIVSAWDAHERRLKNDKQLKVFNCCSGLQNPIDTGNMMSICLEHNKKHEKDKSKANTMFIIRKNFYVYFFYFLVLHLIPALIIDVFYFLLGREMLMCTNLKKIKRFSSILKVFCFNQFLFIDKNVRRLHQALNETDKVLFDFDVRNIQWRVYLKDFVIALKEYDKTSRTVKI

SEQ ID NO: 55 - protein, 454 aa, Spodoptera littoralis
MVVLTSKEKSNMSVADFYAGKSVFITGGTGFLGKVFIEKLLYSCPDIDKIYMLIREKKGQSIRERLTKIVDDPLFNRLKDKRPGDLGKIVLIPGDITVPGLGISEENETILTEKVSVVIHSAATVKFNEPLATAWNVNVEGTRMIMALSRRMKRIEVFIHISTAYTNTNRAVIDEVLYPPPADINDVHQHVKNGVTEEETEKILNGRPNTYTFTKALTEHLVAENQSYMPTIIVRPSIVGAIKDDPIRGWLANWYGATGLSVFTAKGLNRVIYGHSNHVVDLIPVDYVANLVIVAGAKTYHSNEVTIYNSCSSSCNPITMKRLVGLFIDYTVKHKSYVMPLPGWYVYSNYKWLVFLVTVIFQVIPAYLGDIGRRLLGKNPRYYKLQNLVAQTQEAVHFFTSHTWEIKSKRTSELFSSLSLTDQRMFPCDANRIDWTDYITDYCSGVRQFLEKIK

SEQ ID NO: 56 - protein, 454 aa, Spodoptera exigua
MVVLTSKEKSNMSVADFYAGKSVFITGGTGFLGKVFIEKLLYSCPDIDKIYMLIREKKGQSIRERLTKIVDDPLFNRLKEKRPGDLDKIVLIPGDVTVPGLGISDENEAILIDKVSVVIHSAATVKFNEPLETAWNVNVEGTRMIMALSRKMKRIEIFIHISTAYTNTNRAVVDEVLYPPPADINEVHQYVKNGITEEETEKILNGRPNTYTFTKALTEHLVAENQAYMPTIIVRPSIVGAIKDDPIRGWLANWYGATGLSVFTAKGLNRVIYGQSSHVVDLIPVDYVANLVIVAGAKTYRSNEVTIYNSCSSSCNPITMERLVGLFIDDTVKHNSYVMPLPGWYVYSNYRWLVYLVTIIFQMIPAYLADIGRRLLGKNPRYYKLQSLVAQTQEAVHFFTSHTWEIKSKRTSELFASLSHTDQRIFPCDAKKIDWTDYITDYCSGVRQFLEKKK

SEQ ID NO: 57 - protein, 459 aa, Spodoptera exigua
MTYRQINEFDAEKFTAAAVPTSYVSVPDFYAGKSIFITGGTGFLGKVFLEKLLYSCKDVETVYILIREKKGKTPQQRVEDLFNKPIFSRLKQKDSQCMKKVTAIIGDLSEPGLGISKDDEELLLQKVSVVFHVAANVQFYKEFKEIINTNVGGTKYVLQLCQRIKDIKAFVHISTAYCHTDQKVLEERIYPPPAELSEVLKFLQQPQHDKKQIKELFKKQPNSYTFAKALAETYIAENCGRVPTIIIRPSIISASLKEPLPGWVDSWNGATGLITASYNGANRVLLGEGGNFLDLIPVDFVANLAIVAAAKCTSSLKVYNCCSSGCNPLTLKQLVSHMNNVGFDKNVSIIFTNNKASLSTLTFFLQTTPSFTADMFLRVTGKSPRYMKIQSKLTIARNALNFFTCHSWVMKADNSRRLYASLSLHDRHTFPCDPTDIDWKKYITIYIEGINQFLMKKRS

SEQ ID NO: 58 - protein, 450 aa, Spodoptera frugiperda
MSKANCSNVASFYAGKSVFITGGTGFVGKTLIEKLLYSCSGIDKIYVLVRDKYGKQASERLARVTSTPVFDRLKTSKSEELKKIVVISGDITRENLGLQEDVLKTLENEVSVVFHLAATVAFKLPLKDAMRINVNGTENVIELCRRMKKLQAFVHVSTAFTNSDRKEINEMVYPMPINLEEARHVAYTYSHDDVIVSAFMGKKPNTYTFSKAFAEQQVMKQCEDLPTAIIRPSIVMSSVKEPCPGWIDTWNGSTGLFVGMPAGILRVVLGRGSNVTDLVPVDIVTNLIVVAATECQKSKEIKVYNCCTGTSNPITCDEATTISRKVALKYSLNELPWPFLMFTPSVFLYTLITFLLQIVPAYLIDLWCILTGRKATQIKLQRRLKKVVDAVKFFLLNEWKFSDDNIRDLLKTMSPVDKEIFNFDVKTINWETCIEDYILGARKYLLKSEK

SEQ ID NO: 59 - protein, 468 aa, Plutella xylostella
MKLGTFGNMAANGGSVCDFYANSTVFITGGTGFLGKVLIEKLLRSCRKIDKIYVLLRGTTNKTAQERLEDLSHEVLFEKVRQDDPNIMKKLIPVNGDLTLPNLGMNNEDYSLIVKNVSIVFHAAASIRFNLKIKEALHCNVEGTKKVIELCHKARCIKAFVHISTAYSNTDKTFVKEEVYPAPENLDKIYVIIGQNGDDTEADDLTSSRPNTYTYAKAVAEYEVLRNHGSVPTAIIRPSIVTPIWRDPTPGWLDKWSGGTPIIFGLCKGLLRCLDTQPSNVIDFIPADIAVNLSIVAAAKCHVDTPKIRVYNCCSGPTNPMTFKMLSDYCHENSIKHGLHWFPYPKIFLSTNRVFLKIVRLVLQFLPLFLADMWLKIRGKQPRFVKMLEKLTKVEKTLHFFIKNQWQFENHNVEALISTLSPSDRVQFPCDPSEICWKTHFEDYCLGIDKYLMNTSVKSANINSGMYK

SEQ ID NO: 60 - protein, 535 aa, Plodia interpunctella
MRLVPSALDVVLAAERRLVPIHEATLKGDSEVQKFYAGSVVLVTGGSGFLGKQLIEKLFRTCDVKKLYILLRAKKGKSIGERLDYILRNPVYDTLRETRPWFADRIEAVEGDIASYRLGVADHMWDRLVDEVTVVFHGAATINFADPLQLATNINVKGTLEMLEFGKACKNLRSYVHISTAYNQALKNNVSCDILEEFPKAPLSPTTLVDLANRTETKVLDEMFHKYLAPEYPNTYAYTKAVAEEAVRTMAGDLPICVVRPSIVIPAYTEPSAGWVDKNSIYGASGIIMGLGLGVIHTLISDPDIKIDIIPVDIVNNAVIVAGWETARRREQGETDTKIYTVGSSNRNHITWRFLTNVFDNEGRKHLSSQAVWYAFAVQTKSKHLYLALAWLLHFIPGYIVDGVLMIRGKKPQVSKIYRLLNTMATIFSFFTLRGWNFKDDNLLQLYKSLSKTDQQIFNMDIATLDMKEVIYLWHIGLRRFYLKDKLNDQEKSMKKQFFLRIITYTFIPIYLYFLFKISCFVFYGLYYLIFSYLI

SEQ ID NO: 61 - protein, 543 aa, Amyelois transitella
MAAKCEIGVSQFFAGKHVFITGATGFMGKVLVERLLSTCADVGRIYLLMRSKKDHSPEQRLQELKASQAFDNLRQSQPSQLEKLRIVAGDIMLPGLGISQEGLEDLQEVSVVFHSAATVKFNEALKIAIEQNLMSVIRLLEICDRLPHMQAFVHVSTAYSNPELSTVEERVYPPPVPLDYLLDLAAEDSPLLDNVESFISPKPNSYTFTKAMAEAAVQRHGCQYYPIAIVRPTIVISSLKHPFPGWLENANGPTGVIFGAGRGLLRVFRCRQSALADLLPVDIGIDTLIAAAWEVASDRPPSPRVYNCSISENPTTWGDLKRAINLHTRSQPQCGAVWYPGLHTAENGFLYKILEVLLQTTPLYIGEILVRILGYSHKRLNLIRVSTQMKNMQNAMKYFALREWRFQSDNVRRLRERMSDVDKRIYNLDPCSIVWDEHYSNYVKGVRLYLLREKELDQPAAKKRLKKLYYLHYGLSVFMPFLLFRAFVRRKHRQNILEVLFAILSIFNAIFRRSTQNILYIYKVSLTFLNDNVFLNKVFNESS

SEQ ID NO: 62 - protein, 462 aa, Ostrinia zea
MAANTIETDEQFTDNSPIVNFYSGKSVFVTGATGFVGTVLVEKLLFSCKGINNIYILIKQTEDLTIEARILNYLNSKPFHRVKNTNPELLKKIIPICGNLEDKNLGISDSDMKTLLEEVSIVFHVAAKLLFKMNLTAAVNINTKPTEQLIAICKKMRRNPIFIYVSSAYSNVNEQIIDEKVYSTRVPLETVYDTLDIENTRITDIFLDKRPNTYTYSKALAEVVVEKEFNESAAIVRPSIILSSIREPIPGWLSGSHGFPRVVGAACKGLLLRWHGDGTVVCDVIPVDHVANLSIAVAWESNERRLMGNKGVKVYNCCSSLRNPIDVSTVLNTCLKYRKYFGTRTMSIFTPRFIMKKNYFLYKLLYFTYHTIPAAIIDGFFWLTGRTPIMLKTLDKLRKLSSVLEYFTHHQFLFLDSNVRGLLRRMEGTDRQTFNFDVTEIEWEPYLQNFVRGIANNYDNSM

SEQ ID NO: 63 - protein, 453 aa, Trichoplusia ni
MIAVTSHENEASIVDFYEGKSVFITGGTGFLGKVFIEKLLYCCPGLVKIYMLIREKKGLTIKERIDRFVDDSLFDRLKTQRPKDLDKIVLIPGDITAPSLGISQEQENILIEEVSVIIHSAATIKFNAPLEEAWSINVDGTRRILDLCRNMKKVEVFLHISTAYTNINRNVIEEVLYPPPADIKELEKLDKHNVTEKQTMKLLNGFPNTYTFTKSLTEHLVAENCAYMPTIIVRPSVVAAVLEDPIKGWLENWYGATGISVFTAKGLNRVFLGHKNIIVDVIPADYVANLVIIAGARNNKSKELKIYNCCSSGCNPLTIGNLLGAFIDDAIRHKTYAMPLPGWFIFTRYRLVATIVTFIFQVVPAYLADLYRRIIGKKPKYTKLLYLVVQTRVALDFFSSNSWVMKADRMRELYASLSNNDKRMFPCDPTHINWPEYLSDYGAGVRRFLEKRN

SEQ ID NO: 64 - protein, 461 aa, Trichoplusia ni
MFTSTNNIRTEDDVNAEHVESIADFYAGKSVFITGVTGFLGKVYLEKLLYTCKDIDKVFILIRNKKGDDVGKRIEKLLDSSVFSRLKSQRPQDIKKIVPVYGDMDKSELGLSSEDQERLINEVSVVFHVAASVNLDADLRSSMATNFFGTSYVLKLCHRMKKIKVFVYVSTAYCNSTLKVLEEKVYPVQEELEEVLKYLEEPHLDRDRVKKLLNGRPNAYALSKALAEHYVAQNHGDIPIIFIRPSIVISSSKEPVPGWVDSFQGATALITAVLKGVNRVIYGNKNNVLDLIPVDYVSNLSIVAAARVKSSKEVTVYNCCTSSIKPITLKELASQITRRKVEDKQRFLFLPTVFFSTHWLVVIILTFVMQIMPSFIADQFLYFTGNKPMYMKIQSKVINGRNVLNYYTNNSWVFKSVASRRLLDTLSLSDKRLFPCYPSDLNWDKYFKTYFDGIENFLLKK

SEQ ID NO: 65 - protein, 450 aa, Chrysodeixis includens
MSSLKGPKVASFYTGKSVFITGGTGFLGKTLIEKLLYSCSGIDKLYILVRDKRGYAAHERLARITNTPAFDRLKGTRNEDLKKITVITGDIARPNLGLPDDILKRLEDEVSVVFHLAATVAFNLRLKDAMGININGTEEVVKLCHRMKKLQAFVHVSTAFSNSDRFEIEEKVYPMPVTLEDARHVAEMYSHDDEVITQFLGKKPNTYTFSKALAEDLVMKQCQDLPAAIVRPSIVMSSIKEPCPGWIDTWSGSTGLFVGMPAGVLKVVKGKGSNVTDLVPVDIVTNFLIVVATECQKTNEIKVYNCCTGTSNPITSNYASAIARRVTLRYSLNELPWPFLVFTSSLIVYTLITFMLQTIPAFLIDLWCVLTGKKATQLKLQSRLKKNIEVVKFFLLNEWKFCDENVRNLFKSLSPEDKETFNFDVKSINWEKSIEDYILGARKYLLKYEK

SEQ ID NO: 66 - protein, 461 aa, Chrysodeixis includens
MFASTDGGRSDEETPQQHESIADFYAGKSVFITGVTGFLGKAYLEKLLYSCKDIDKVFVLIRNKRGDDVGKRIEKLLDSSVFSRLRSHRPEDLKKITPVYGDIDKSELGLSLADQERLIKEVSVVFHVAASVNLDADLKTSLVTNYFGTTHVLKLCHRMKKLKVFVYVSTAYCNTTVKVLEEKVYPLPVELDEVVKIMEQPHLDSNRVKKLLNGRPNTYALSKALAEHYIAENHGDIPVIFIRPSIVTSSAREPAPGWADSFQGATALITACWKRVNRVIYGNGDNIIDLIPVDYVSNLSIVAAAKVKSTTEIAVYNSCTSSIKPMTLKGIAEQIERVNTENKQGSIFLPTIFFTSSWLFVIMLTFVLQLLPSFIADLFLYITGNKPMYMKIQSKVLHGRHILNYYTNDSWVFKSDSSQRLQDSLSPADKRLFPCCPSDIKWDEYFNTYFCGIDNYLLKRK

SEQ ID NO: 67 - DNA, 54 bp, synthetic construct
  misc_feature 1..54: note = Artificial
  source 1..54: mol_type = other DNA
  modified_base 8: mod_base = OTHER; note = Uracil
cacgcgatatggtctcacagagaagacttcagacgatttgtcttagaggaacgc

SEQ ID NO: 68 - protein, 519 aa, synthetic construct
  REGION 1..519: note = Artificial
MSTFDWESIVPATPLDQIPGDIQRLRKGFRSGKTLDLNYRLDQIRNLHYVLRDNVEAIKDAVYKDLGRPKHETDLCEVGFLWGEFNNVVANLKKWAADEDVKTNLQYSISSPKIRKRPLGNVLIISPWNYPFMLTVSPLIGALAAGNTVAVKFSEMAPHTSKIVGDLCTKALDPDVFQAIQGGVPVVTKTLEQKFDKIMYTGNHTVGKIIATAANKYLTPVILELGGKSPVFVTKNCKNIKLAAKRALWGKVVNAGQTCVAPDYVIVEPEVEQEFIDACKYWINEFYSGKIDQYNPDFAKIATPNHWNRLTSMLSKSKGEIITGGNTDEKTRFIAPTVVAKVPDNDSLMEDEIFGPLLPILTARSVEEGIKYVHENHDTPLAMYVFTDKASEGDYIQSQINSGGLIFNDTLIHVGCVQAPFGGVGMSGYGAYHGEDSFLAFTHRQTYLNQPKLLEPLQDVRYAPYTKTKRSMVKNLLLVGPIFPRTGSVYPNVLIRIFRKIWFWVLIVAIGAAGAKALL

SEQ ID NO: 69 - protein, 377 aa, synthetic construct
  REGION 1..377: note = Artificial
MWGSSHAFAGESDLTLQLHTRSNMSDNTTIKKPIRPKPIRTERLPYAGAAEIIRANQKDHYFESVLEQHLVTFLQKWKGVRFIHQYKEELETASKFAYLGLCTLVGSKTLGEEYTNLMYTIRDRTALPGVVRRFGYVLSNTLFPYLFVRYMGKLRAKLMREYPHLVEYDEDEPVPSPETWKERVIKTFVNKFDKFTALEGFTAIHLAIFYVYGSYYQLSKRIWGMRYVFGHRLDKNEPRIGYEMLGLLIFARFATSFVQTGREYLGALLEKSVEKEAGEKEDEKEAVVPKKKSSIPFIEDTEGETEDKIDLEDPRQLKFIPEASRACTLCLSYISAPACTPCGHFFCWDCISEWVREKPECPLCRQGVREQNLLPIR

SEQ ID NO: 70 - protein, 609 aa, synthetic construct
  REGION 1..609: note = Artificial
MSDDKHTFDFIIVGGGTAGPTLARRLADAWISGKKLKVLLLESGPSSEGVDDIRCPGNWVNTIHSEYDWSYEVDEPYLSTDGEERRLCGIPRGHCLGGSSCLNTSFVIRGTRGDFDRIEEETGAKGWGWDDLFPYFRKHECYVPQGSAHEPKLIDFDTYDYKKFHGDSGPIKVQPYDYAPISKKFSESLASFGYPYNPEIFVNGGAPQGWGHVVRSTSNGVRSTGYDALVHAPKNLDIVTGHAVTKILFEKIGGKQTAVGVETYNRAAEEAGPTYKARYEVVVCCGSYASPQLLMVSGVGPKKELEEVGVKDIILDSPYVGKNLQDHLICGIFVEIKEPGYTRDHQFFDDEGLDKSTEEWKTKRTGFFSNPPQGIFSYGRIDNLLKDDPVWKEACEKQKALNPRRDPMGNDPSQPHFEIWNAELYIELEMTQAPDEGQSVMTVIGEILPPRSKGYVKLLSPDPMENPEIVHNYLQDPVDARVFAAIMKHAADVATNGAGTKDLVKARWPPESKPFEEMSIEEWETYVRDKSHTCFHPCGTVKLGGANDKEAVVDERLRVKGVDGLRVADVSVLPRVPNGHTQAFAYAVGEKAADLILADIAGKDLRPRI

SEQ ID NO: 71 - protein, 727 aa, synthetic construct
  REGION 1..727: note = Artificial
MSETDHLLAAEPVAEYPQYTPWPNSRKSVDTEFSATSWIYDLVLWIFTACFDIFFREIRPRGAFRIPRKGPVLFVAAPHANQFVDPVILMNQVKQEAGRRISFLVAEKSMRRAAVGRMARSMNSIPVVRAQDNAKKGEGKIYVDAEDPTKIHGIGTQFTKQCEVRGLVVCSSSVGSIDVAEIVSDTLLIARKEFKGPKAKEALKESNGGITYKYADYVNQATVYRSVFDKLHHGGCVGIFPEGGSHDRTELLPLKAGVAIMALGALAEDPSCGVRIVPCGLNYFHAHKFRSRAVVEFGSPIAIPPDLVEKYKAGGEAKREAVKTVLDITAAGLKSVTVQVQDFDTLMLIQAIRRLYRPPGKKIPLPMVVELNRRLVYAYNHYKDDPRIEEMKQEIRKYNKFLQAMGLKDHQVEKARISKIEILGRLLYRSIKLVFLSIGCLPGLLLFSPIFIISKSISKTKAKEALKASSVKIKANDVVATWKVLVAMGLTPVLYILYSLVGSVVIRKLDLISWFPTILLPGLVLSIIITTSYAALAMGEAGMDIFKSLRPLALALNPSTKNSLLKLQNERKRLVLKSSELVTSLGPELFPDFPENSILQGSDKFEDEENYENEKRSHSRSTSATSLSAMSEGDGDELVREVRKGASYFPVSTISEDEDQAISRVGSEASLADIPLFGMSRSQSGASLSEASTHGSSTGADAEEAKTEVTRRIALAMEEKRREQDEE

SEQ ID NO: 72 - protein, 326 aa, Amyelois transitella
MVPNKGSSDVLSEHSEPQFTKLIAPQAGPRKYKIVYRNLLTFGYWHLSAVYGLYLCFTCAKWATILFAFFLYVIAEIGITGGAHRLWAHRTYKAKLPLEILLLIMNSIAFQDTAFTWARDHRLHHKYSDTDADPHNATRGFFYSHVGWLLVKKHPEVKARGKYLSLDDLKNNPLLKFQKKYAILVIGTLCFLMPTFVPVYFWGEGISTAWNINLLRYVMNLNMTFLVNSAAHIFGNKPYDKSIASVQNISVSLATFGEGFHNYHHTYPWDYRAAELGNNRLNMTTAFIDFFAWIGWAYDLKSVPQEAIAKRCAKTGDGTDMWGRKR

SEQ ID NO: 73 - DNA, 981 bp, Amyelois transitella
  source 1..981: mol_type = other DNA
atggtgcccaacaagggttcttccgacgtcctgtctgagcactccgagccccagttcaccaagctgattgctccccaggctggcccccgaaagtacaagatcgtgtaccgaaacctgctgaccttcggatactggcacctgtctgccgtctacggtctgtacctgtgtttcacctgcgccaagtgggctaccattctgttcgccttcttcctgtacgtgatcgctgagatcggcattaccggcggagcccaccgactgtgggctcaccgaacctacaaggccaagctgcccctggagatcctgctgctgattatgaactctatcgctttccaggacaccgccttcacctgggctcgagatcaccgactgcaccacaagtactctgacaccgacgctgaccctcacaacgctacccgaggtttcttctactcccacgtgggctggctgctggtcaagaagcaccccgaggtgaaggcccggggaaagtacctgtccctggacgacctgaagaacaaccccctgctgaagttccagaagaagtacgctatcctggtcattggcaccctgtgtttcctgatgcccaccttcgtgcccgtctacttctggggtgagggcatttctaccgcctggaacatcaacctgctgcgatacgtgatgaacctgaacatgaccttcctggtcaactccgccgctcacattttcggcaacaagccctacgacaagtctattgcctccgtgcagaacatctctgtctccctggctaccttcggagagggtttccacaactaccaccacacctacccttgggactaccgagctgctgagctgggcaacaaccgactgaacatgaccaccgccttcattgacttcttcgcctggatcggatgggcttacgacctgaagtccgtcccccaggaagccatcgctaagcgatgcgctaagaccggcgacggaaccgacatgtggggacgaaagcgatag

SEQ ID NO: 74 - protein, 338 aa, Spodoptera exigua
MAQTIQTTTILEQKEEKTVTLLVPQAGKRKFEFVYQNLITFAYWHIAGLYGLYLCFTSAKWATILFSFILFVIAEIGITAGAHRLWSHKSYKVKLPLEILLMVMNSIAFQNTVIDWVRDHRLHHKYSDTDADPHNASRGFFYSHIGWLFVRKHPEVKKRGKELDMSDIYNNPVLRFQRKYAVPFIGAVCFGLPTLIPVYCWGESWTNAWHITMLRYIMNLNATFLVNSAAHIYGKRPYDKKILPAQNIGVSIATFGEGFHNYHHVFPWDYRAAELGNNGLNLTTKFIDFFAWIGWAYDLKTVSKEMIKQRSKRTGDGTNLWGLEDKDTPENLKNIKGE

SEQ ID NO: 75 - DNA, 1017 bp, Spodoptera exigua
  source 1..1017: mol_type = other DNA
atggctcagaccatccagaccaccaccatcctcgagcagaaggaggagaagaccgtgaccctgctggtcccccaggctggtaagagaaagttcgagttcgtctatcagaacctgattacttttgcctactggcatatcgctggcctgtacggtctttacctgtgcttcacctccgccaagtgggccaccattctgttctccttcatcctgttcgtcatcgccgagattggaatcaccgccggagctcaccgactgtggtcccacaagtcttacaaggtcaagctcccccttgagatccttctgatggtcatgaactccattgccttccagaataccgtcattgactgggtccgagaccaccggcttcaccataagtactccgacaccgatgccgaccctcataacgcctcccgaggcttcttctactcccacatcggctggctgttcgtgcgaaagcaccccgaggtcaagaagcgaggtaaggagctggacatgtccgacatctacaataaccccgtcctgagattccagcggaagtacgccgttcctttcattggcgctgtctgcttcggcctgcccacccttatccccgtctattgctggggcgagtcctggaccaacgcctggcacatcaccatgctgcgatacatcatgaacctcaatgccaccttcctggtcaactctgccgcccacatctacggaaagcgaccctacgacaagaagatccttcccgcccagaacatcggcgtctccattgccacctttggtgagggtttccacaactaccaccacgtgttcccttgggattaccgagctgccgagctgggcaacaacggtctgaacctgaccaccaagttcattgacttcttcgcttggatcggatgggcctacgacctcaagacggtctccaaggagatgatcaagcagcgatccaagcgaactggcgacggaactaacctgtggggcctggaggacaaggatactcctgagaacctgaagaacatcaagggcgagtaa

SEQ ID NO: 76 - protein, 338 aa, Spodoptera litura
MAQTIQTTTILEQKEEKTVTLLVPQAGKRKFEIVYFNLVSFAYWHIAGLYGLYLCFTSAKWATILFSFFLFVVAEVGVTAGAHRLWSHKTYKAKLPLQILLMVMNSLAFQNTAIDWVRDHRLHHKYSDTDADPHNASRGFFYSHIGWLFVRKHPDVKKRGKEIDISDIYNNPVLRFQKKYAIPFIGAVCFALPTLIPVYGWGETWTNAWHVAMLRYIMNLNVTFLVNSAAHIYGKRPYDKKILPSQNIAVSIATFGEGFHNYHHVFPWDYRAAELGNNCLNFTTKFIDFFAWIGWAYDLKTVSKEMIKQRSKRTGDGTNLWGLEDVDTPEDLKNTKGE

SEQ ID NO: 77 - DNA, 1017 bp, Spodoptera litura
  source 1..1017: mol_type = other DNA
atggctcagaccatccagaccaccaccatcctcgagcagaaggaggagaagaccgtgaccctgctggtccctcaggctggtaagagaaagttcgagatcgtctatttcaacctggtctcttttgcctactggcatatcgctggcctgtacggtctttacctgtgcttcacctccgccaagtgggccaccattctgttctccttcttcctgttcgtcgtcgccgaggttggagttaccgccggagctcaccgactgtggtcccacaagacttacaaggccaagctcccccttcagatccttctgatggtcatgaactccctggccttccagaataccgccattgactgggtccgagaccaccggcttcaccataagtactccgacaccgatgccgaccctcataacgcctcccgaggcttcttctactcccacatcggctggctgttcgtgcgaaagcaccccgatgtcaagaagcgaggtaaggagattgacatctccgacatctacaataaccccgtcctgagattccagaagaagtacgccatccctttcattggcgctgtctgcttcgccctgcccacccttatccccgtctatggttggggcgagacctggaccaacgcctggcacgtcgccatgctgcgatacatcatgaacctcaatgttaccttcctggtcaactctgccgcccacatctacggaaagcgaccctacgacaagaagatccttccctctcagaacatcgccgtctccattgccacctttggtgagggtttccacaactaccaccacgtgttcccttgggattaccgagctgccgagctgggcaacaactgcctgaacttcaccaccaagttcattgacttcttcgcttggatcggatgggcctacgacctcaagacggtctccaaggagatgatcaagcagcgatccaagcgaactggcgacggaactaacctgtggggcctggaggacgtcgatactcctgaggacctgaagaacaccaagggcgagtaa

SEQ ID NO: 78 - protein, 338 aa, Helicoverpa zea
MAQSYQSTTVLSEEKELTLQHLVPQASPRKYQIVYPNLITFGYWHIAGLYGLYLCFTSAKWATILFSYILFVLAEIGITAGAHRLWAHKTYKAKLPLEILLMVFNSIAFQNSAIDWVRDHRLHHKYSDTDADPHNASRGFFYSHVGWLLVRKHPEVKKRGKELNMSDIYNNPVLRFQKKYAIPFIGAVCFALPTMIPVYFWGETWSNAWHITMLRYIMNLNVTFLVNSAAHIWGNKPYDAKILPAQNVAVSVATGGEGFHNYHHVFPWDYRAAELGNNSLNLTTKFIDLFAAIGWAYDLKTVSEDMIKQRIKRTGDGTDLWGHEQNCDEVWDVKDKSS

SEQ ID NO: 79 - DNA, 1017 bp, Helicoverpa zea
  source 1..1017: mol_type = other DNA
atggcccagtcttaccagtctaccaccgtgctgtctgaggaaaaggaactgaccctgcagcacctggtgcctcaggcttcgccccgaaagtaccagatcgtgtaccccaacctgatcaccttcggctactggcacattgccggcctgtacggactgtacctgtgcttcacctctgccaagtgggccaccatcctgttctcttacattctgttcgtgctggccgagatcggcatcaccgctggcgcccaccgactgtgggctcacaagacctacaaggccaagctgcccctcgagatcctgctgatggtgttcaactctatcgccttccagaactctgccatcgactgggtgcgagatcaccgactgcaccacaagtactctgacaccgacgctgaccctcacaacgcctctcgaggcttcttctactctcacgtcggctggctgctggtgcgaaagcaccccgaggtgaagaagcgaggcaaggaactcaacatgtctgacatctacaacaaccccgtgctgcgattccagaagaagtacgctatccccttcatcggcgccgtgtgcttcgctctgcccactatgatccccgtgtacttctggggcgagacttggtctaacgcctggcacatcaccatgctgcggtacatcatgaacctgaacgtgaccttcctggtgaactctgctgcccacatctggggcaacaagccctacgacgccaagattctgcccgctcagaacgtggccgtgtctgtggccaccggcggagagggcttccacaactaccaccacgtgttcccttgggactaccgagccgccgagctgggcaacaactctctgaacctgaccaccaagttcatcgacctgttcgccgccatcggctgggcctacgacctcaagaccgtgtccgaggacatgatcaagcagcgaatcaagcgaaccggcgacggcaccgacctctggggccacgagcagaactgcgacgaggtgtgggacgtgaaggacaagtcctcttaa

SEQ ID NO: 80 - protein, 344 aa, Diatraea saccharalis
MAPNLIKNEGTFDMDIKPDELQLPKFSNIKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFANIGWAYDLKTVSNEMARARAMRTGDGKDLWGWDEKDVTDKDKEHTGILNGKTD

SEQ ID NO: 81 - DNA, 1035 bp, Diatraea saccharalis
  source 1..1035: mol_type = other DNA
atggctcccaacctgatcaagaacgagggcaccttcgacatggacatcaagcccgacgaactgcagctgcccaagttctctaacattaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgccaacatcggctgggcctacgacctcaagaccgtgtctaacgagatggcccgagccagagccatgcgaaccggcgacggcaaggacctgtggggctgggacgagaaggacgtgaccgacaaggataaggaacacaccggcatcctgaacggcaagaccgactag

SEQ ID NO: 82 - protein, 352 aa, Diatraea saccharalis
MAPNATDVNGVLFEDDAATPDMALPNLPVQKADNYPKKFVWRNIIAFAYLHLAAIYGGYLFLFSAKWQTDIFAYILYVMSGLGITAGAHRLWAHKSYKAKWPLRLILVFFNTLAFQDSAIDWSRDHRMHHKYSETDADPHNATRGFFFSHIGWLLVRKHPELKKKGKGLDLSDLYADPILCFQKKYYLILMPLCCFVMPTVIPVYFWGETWTNAFFVAALFRYTFILNVTWLVNSAAHKWGHKPYDKNIKPSENLSVAIFALGEGFHNYHHTFPWDYKTAELGNNRLNFTTNFINFFAKIGWAYDMKTVSDEIVQKRVNRTGDGSHHLWGWGDKDQPKEEVDAAIRINPKDD

SEQ ID NO: 83 - DNA, 1059 bp, Diatraea saccharalis
  source 1..1059: mol_type = other DNA
atggctcccaacgccaccgacgtgaacggcgtgctgttcgaggacgacgccgctactcccgacatggctctgcccaacctgcctgtgcagaaggccgacaactaccccaagaagttcgtctggcgaaacatcattgccttcgcctacctgcacctggccgccatctacggcggctacctgttcctgttctctgccaagtggcagaccgacattttcgcctacatcctgtacgtgatgtctggcctgggcatcaccgctggcgcccaccgactgtgggctcacaagtcttacaaggctaagtggcccctgcgactgatcctggtgttcttcaacaccctggccttccaggactctgccatcgactggtcccgagatcaccgaatgcaccacaagtactctgagactgacgctgaccctcacaacgctacccgaggcttcttcttctctcacatcggctggctgctggtgcgaaagcaccccgagcttaagaagaagggcaagggcctcgacctgtctgacctgtacgctgatcccatcctgtgcttccagaagaagtactacctgattctgatgcccctgtgctgcttcgtcatgcccaccgtgattcccgtgtacttctggggcgagacttggaccaacgccttcttcgtggccgctctgttccgatacaccttcattctgaacgtgacctggctggtgaactctgctgcccacaagtggggacacaagccctacgacaagaacatcaagccctctgagaacctgtctgtggccatcttcgccctcggcgagggattccacaactaccaccacacattcccttgggactacaagaccgccgagctgggcaacaaccgactgaacttcactaccaacttcatcaactttttcgccaagatcggctgggcctacgacatgaagaccgtgtctgacgagatcgtgcagaagcgagtcaaccgaaccggcgacggctctcaccacctgtggggctggggcgacaaggaccagcctaaggaagaggtggacgccgccatccgaatcaaccccaaggacgactag

SEQ ID NO: 84 - protein, 679 aa, Cydia pomonella
MADSPPEDNTPTRNGSLKISLPTVLCTPESSENSDILCKTKTFISSESKLSLPSNLGTPTSPLRLSSPLSLPTSPTPYMKITQPQPEKTVAATQMNSPPTTIVQQTGPKQPLVNTGSATGNPRNKCALQPGHSLMDWIRLGNSGKDLTGVGGRMRPVSPDELAAHNTRNDAWLAIRGRVYNITHYLPYHPGGPEELMRGAGIDATQLFDKVHPWVNYDSLLAKCLVGPLRLDRPDAEELFGPSTPSPKSENRLREPSKAQELVRKSMENLANCITPVRKKMSAKSEENIKGSPPSKIMQSLIQSSDLPVSISRRAAASPVKTSEKSDIAPSPLRFDWIQTSTKLTLSIYTGPLANPGGCARIIEGILFVEVATNGWVRTLKLVPEAKLLESLQLRVFAESGKIEVTAHKAEPGVWKTCGEAMAGAASRVSTPRSVSCRVARMLRVSHDTVLLSLAAPAQALVAPLGHHVRVHRTISDVECVRSYTPVGEGWGPDAERDSAIHLAVKGYDTGALSPYLTALQPGDEVTVSGPYGNFQLHTLKGVKEIYFVAAGTGITPMLGLLKFMLPRSNPRCERIHLLFFNKTEEDILFRENFEEIAREDDRFTVTHVLSDAGPSWSGHKGRVTTNLLTQVIGKSFLKCRDQCTHFACVCGPTEFTHTAIDLLKKLDMKDSCIHAFMG

SEQ ID NO: 85 - DNA, 2040 bp, Cydia pomonella
  source 1..2040: mol_type = other DNA
atggccgactctccacctgaggacaacacccctactcgaaacggctctctgaagatctctctgcccaccgtgctgtgcacccctgagtcctctgagaactctgacatcctgtgcaagaccaagaccttcatctcttctgagtctaagctgtctctgccctctaacctgggaacccctacctctcctctgcgactctcttctcccctgtcgctgcccacctctcctacaccttacatgaagatcacccagcctcagcctgaaaagaccgtggccgccactcagatgaactccccacctaccaccatcgtgcagcagaccggacctaagcagcccctggtgaacaccggctctgccactggcaaccctcgaaacaagtgcgctctgcagcccggccactctctgatggactggatccgactgggcaactctggcaaggacctgaccggcgtcggcggacgaatgcgacccgtgtctcccgacgagctggccgctcacaacacccgaaacgacgcctggctggccatccgaggccgagtgtacaacatcacccactacctgccttaccatcctggcggacccgaggaactgatgcgaggcgccggaatcgacgctacccagctgttcgacaaggtgcacccttgggtgaactacgactctctgctggccaagtgcctggtgggacccctgcgactggaccgacctgacgctgaggaactgttcggcccctctactccctcgcctaagtctgagaaccgactgcgagagccctctaaggcccaagagctggtgcgaaagtctatggaaaacctggccaactgcatcacccctgtgcgaaagaagatgtctgccaagtctgaggaaaacatcaagggatctccaccttctaagatcatgcagtctctgattcagtcctccgacctgcctgtgtctatttctcgacgagccgccgcttctcccgtcaagacctccgagaagtctgatatcgccccttcgcctctgcgattcgactggattcagacctctaccaagctgaccctgtctatctacaccggacctctggctaaccccggtggctgcgcccgaatcatcgagggcatcctgttcgtcgaggtcgccaccaacggctgggtgcgaaccctgaagctggtgcccgaggccaagctgctcgagtctctgcagctgcgagtgttcgccgagtctggcaagatcgaggtgactgcccacaaggctgagcccggcgtgtggaagacctgcggcgaggccatggctggcgctgcctctcgagtgtctacccctcgatctgtgtcttgccgagtggcccgaatgctgcgagtctcccacgacaccgtcctgctgtccctggctgctcccgctcaggccctggtggctcccctgggccaccacgtgcgagtgcaccgaaccatctctgacgtcgagtgcgtgcgatcttacacccctgtcggcgaaggctggggacccgacgccgagcgagactctgccatccacctggccgtgaagggctacgacaccggcgctctgtctccctacctgactgccctgcagcctggcgacgaggtgaccgtgtctggcccctacggcaacttccagctgcacaccctgaagggcgtgaaggaaatctacttcgtcgccgctggcaccggaatcacccctatgctgggcctgctgaagttcatgctgccccgatctaaccctcgatgcgagcgaatccacctcctgttcttcaacaagaccgaagaggacattctgttccgagagaacttcgaggaaatcgcccgagaggacgaccgattcaccgtgactcacgtgctgtctgacgctggcccctcttggtctggccacaagggacgagtgaccaccaacctgctgacccaggtgatcggcaagtctttcctgaagtgccgagatcagtgcacccacttcgcctgcgtgtgcggccccaccgagtttacccacaccgccatcgacctgctcaagaagctggacatgaaggactcttgcatccacgccttcatgggctag

SEQ ID NO: 86 - DNA, 39 bp, Synthetic
  source 1..39: mol_type = other DNA
tatataactgtctagaaataaagagtatcatctttcaaa

SEQ ID NO: 87 - DNA, 1422 bp, Diatraea saccharalis
  source 1..1422: mol_type = other DNA
atggaagagatcgagaacgtcaacgagatcctggtgaagtctcagctgaactctcccgtggtgaacttctactctggccgatctgtgttcattaccggctgtaccggcttcctgggcaccgtgctgctcgagaagctgctgttcacctgtggacacaacctgaagcacgtgtacatcctgatcaagcagaaggacgaccagaacatcgacgagaaggtgtctaacttcttcaactctcgagtgttccagcgactgaacaagcacaaccccaacttccgagccaagatcatccccatctctggcgacctgtctaagaagtgcatcggcatcaacgagaacaacctgtctctgctgcgaaaggaagtgtctgtcgttttccactctgccgccgacatctctttcgacatgtctgtgggcgacgccgtgaacatcaacaccaaggccaccgagaagctcctcaagatctgcaaggaaatgcaccagctgaaggctttcgtgtacgtgtctaccgcctactctaactgcaaccgaaacgtgattgatgagaaggtctacccctctgacgtgcccctcgagactctgtacgaggtcctcgagcactgcaagtctgagcgactgacccagtacctgctgaacggacgacccaacgcctactcgtactctaaggccctgtctgaggaactgatccagaactacgccaactctgtgccctctatcatcattcgaccttctattatcatctcttctgtgaaggaccccatgcctggctggctcggcggctggaacggcctgtctcgagtgattcacgccggcatgaacggcgacatgaagtgctggatcgctgaccctctgtgcaccaccgacatcatccccgtggactacaccgccaacatcatgatcgtgtctgcctgggaggccggtgagaagctgaagtctcgacaccagctcaaggtgtacaactgctgctctggcctgcagaaccccatcaactctggcaccatcgtgaaggactgcctcgagtacaacgaggaatacgagaaggacaagtctaaggtgaacaagatcttcatcatgcggaacaacttctacgtgtacctgatgttcctgttcctgcacatcattcccgccttcgtgatcgacgtgttctacttcctgaccggccgaaagatgatcatgctgaagcgactcaagaagatcaagctgctctctaagatgttcaagtctttctgcgtgaaggaatttctgttcatcgacaacaacgtgcgaaagctgtacgagtctctgaacgagtctgagaagatcctgttcgacttcaacgtgaagaacctgcagtggaaggaatatatgaagtctttcgtcattgccatgaaggaatactccaaggaaactaagatctccaagaagcagaacaagattgagtag

SEQ ID NO: 88 - protein, 473 aa, Diatraea saccharalis
MEEIENVNEILVKSQLNSPVVNFYSGRSVFITGCTGFLGTVLLEKLLFTCGHNLKHVYILIKQKDDQNIDEKVSNFFNSRVFQRLNKHNPNFRAKIIPISGDLSKKCIGINENNLSLLRKEVSVVFHSAADISFDMSVGDAVNINTKATEKLLKICKEMHQLKAFVYVSTAYSNCNRNVIDEKVYPSDVPLETLYEVLEHCKSERLTQYLLNGRPNAYSYSKALSEELIQNYANSVPSIIIRPSIIISSVKDPMPGWLGGWNGLSRVIHAGMNGDMKCWIADPLCTTDIIPVDYTANIMIVSAWEAGEKLKSRHQLKVYNCCSGLQNPINSGTIVKDCLEYNEEYEKDKSKVNKIFIMRNNFYVYLMFLFLHIIPAFVIDVFYFLTGRKMIMLKRLKKIKLLSKMFKSFCVKEFLFIDNNVRKLYESLNESEKILFDFNVKNLQWKEYMKSFVIAMKEYSKETKISKKQNKIE

SEQ ID NO: 89 - DNA, 1035 bp, Diatraea saccharalis
  source 1..1035: mol_type = other DNA
atggctcccaacctgatcaagaacgagggcaccttcgacgtggacatcaagcccgacgaactgcagctgcccaagctgtctaacattaagcccaagatcctgcacgtgaacctgatctacttcctgtactggcacctggctgctccctacggcctgtacctgtgctgcacctctgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccagaagaagtacgctatccccgttttcgcctctttctgctacattctgcccactctgatccccatgtacctctggggcgagtctttcaacaactcttggcacgtcaacctgctgcggtacatcatgaacctgaacgtgaccctgctggtgaactctgccgctcaccgatggggctacaagacctacgaaaagaacatcaaccccactcagaacgtgtacgtgtctttcgtgaccctcggcgagggcttccacaactaccaccacatctttccatgggattacaagaccgctgagctgggaaacaacaagctgaacgtcactacctggttcatcaactttttcgcctggatcggctgggcctacgacctcaagaccgtgtctaacgagatggcccgagccagagccatgcgaaccggcgacggcaaggacctgtggggctgggacgagaaggacgtgaccgacaaggataaggaacacaccggcatcctgaacggcaagaccgactag

SEQ ID NO: 90 - protein, 344 aa, Diatraea saccharalis
MAPNLIKNEGTFDVDIKPDELQLPKLSNIKPKILHVNLIYFLYWHLAAPYGLYLCCTSAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAIPVFASFCYILPTLIPMYLWGESFNNSWHVNLLRYIMNLNVTLLVNSAAHRWGYKTYEKNINPTQNVYVSFVTLGEGFHNYHHIFPWDYKTAELGNNKLNVTTWFINFFAWIGWAYDLKTVSNEMARARAMRTGDGKDLWGWDEKDVTDKDKEHTGILNGKTD

SEQ ID NO: 91 - DNA, 1035 bp, Diatraea saccharalis
  source 1..1035: mol_type = other DNA
atggctcccaacctgatcaagaacgagggcaccttcgacatggacatcaagcccgacgaactgcagctgcccaagttctctaacattaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgcctggatcggctgggcctacgacctcaagaccgtgtctaacgagatggcccgagccagagccatgcgaaccggcgacggcaaggacctgtggggctgggacgagaaggacgtgaccgacaaggataaggaacacaccggcatcctgaacggcaagaccgactag

SEQ ID NO: 92 - protein, 344 aa, Diatraea saccharalis
MAPNLIKNEGTFDMDIKPDELQLPKFSNIKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFAWIGWAYDLKTVSNEMARARAMRTGDGKDLWGWDEKDVTDKDKEHTGILNGKTD

SEQ ID NO: 93 - DNA, 1035 bp, Diatraea saccharalis
  source 1..1035: mol_type = other DNA
atggctcccaacctgatcaagaacgagggcaccttcgacgtggacatcaagcccgacgaactgcagctgcccaagctgtctaacattaagcccaagatcctgcacgtgaacctgatctacttcctgtactggcacctggctgctccctacggcctgtacctgtgctgcacctctgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccagaagaagtacgctatccccgttttcgcctctttctgctacattctgcccactctgatccccatgtacctctggggcgagtctttcaacaactcttggcacgtcaacctgctgcggtacatcatgaacctgaacgtgaccctgctggtgaactctgccgctcaccgatggggctacaagacctacgaaaagaacatcaaccccactcagaacgtgtacgtgtctttcgtgaccctcggcgagggcttccacaactaccaccacatctttccatgggattacaagaccgctgagctgggaaacaacaagctgaacgtcactacctggttcatcaactttttcgccaacatcggctgggcctacgacctcaagaccgtgtctaacgagatggcccgagccagagccatgcgaaccggcgacggcaaggacctgtggggctgggacgagaaggacgtgaccgacaaggataaggaacacaccggcatcctgaacggcaagaccgactag

SEQ ID NO: 94 - DNA, 1557 bp, Tyta alba
  source 1..1557: mol_type = other DNA
atggtgtctatccccgagtactacgagggcaagaacatcctgctgaccggcgccaccggcttcatgggcaaggtgctgctcgagaagctgctgcgatcttgccccaaggtgaaggccgtgtacgtgctggtgcgacacaaggccggacagacccctgaggctcgaatcgaggaaatcaccaactgcaagctgttcgaccgactgcgagatgagcagcccgacttcaaggccaagatcatcgtgatcacctctgagctgacccagcctgagctggacctgtctgagcccatcaaggaaaagctgatcgagcgaatcaacatcatcttccactgcgccgccaccgtgcgattcaacgagactctccgagatgccgtgcagctgaacgtgaccgctactcagcagctcctgttcctggctcagcgaatgaagaacctggaagtgttcatgcacgtgtctaccgcctacgcctactgcaaccgaaagcagatcgaagagatcgtgtaccctcctccagtggaccccaagaagctgattgactctctcgagtggatggacgacggcctggtgaacgacatcacccctaagctcatcggcgaccgacctaacacctacacttacaccaaggctctggccgagtacgtggtgcagcaagagggcgccaagctgaacaccgccatcattcgaccctctatcgtgggcgcctcttggaaggaaccctttcctggctggatcgacaacttcaacggcccctctggcctgttcattgccgccggaaagggcatcctgcgaaccatgcgagcctctaactctgccgtggccgacctggtgcctgtggacgtggtggtgaacaccactctggccgctgcctggtactctggcgtgaaccgacctcgaaacgtgatgatctacaactgcaccaccggcggcactaaccccttccactggggcgaagtgggctaccacatcaacctgaacttcaagatcaaccctctcgagaacgccgtgcgacaccccaactgttctctgcagtctaaccctctgctccatcagtactggaccgccgtgtctcacaccatgcctgcctttctgctggacctcctgctgcgactgaccggacacaagccctggatgatgaagaccatcactcgactgcacaaggccatgatgctcctcgagtacttcacctccaactcttggatctggaacaccgagaacatgaccatgctgatgaaccagctgaaccccgaggacaagaagaccttcaacttcgacgtgcgacagctgcactgggctgagtacatggaaaactactgcatgggcaccaagaagtacgtcctgaacgaggaaatgtctggactgcccgctgccagaaagcacctgaacaagctgcgaaacatccgatacggcttcaacaccgtgctggtcatcctgatctggcgaatcttcattgcccgatctcagatggcccgaaacatctggtacttcgtggtgtctctgtgctacaagttcctgtcttacttccgagcctcttctaccatgcgatactctaagctgtag

SEQ ID NO: 95 - protein, 518 aa, Tyta alba
MVSIPEYYEGKNILLTGATGFMGKVLLEKLLRSCPKVKAVYVLVRHKAGQTPEARIEEITNCKLFDRLRDEQPDFKAKIIVITSELTQPELDLSEPIKEKLIERINIIFHCAATVRFNETLRDAVQLNVTATQQLLFLAQRMKNLEVFMHVSTAYAYCNRKQIEEIVYPPPVDPKKLIDSLEWMDDGLVNDITPKLIGDRPNTYTYTKALAEYVVQQEGAKLNTAIIRPSIVGASWKEPFPGWIDNFNGPSGLFIAAGKGILRTMRASNSAVADLVPVDVVVNTTLAAAWYSGVNRPRNVMIYNCTTGGTNPFHWGEVGYHINLNFKINPLENAVRHPNCSLQSNPLLHQYWTAVSHTMPAFLLDLLLRLTGHKPWMMKTITRLHKAMMLLEYFTSNSWIWNTENMTMLMNQLNPEDKKTFNFDVRQLHWAEYMENYCMGTKKYVLNEEMSGLPAARKHLNKLRNIRYGFNTVLVILIWRIFIARSQMARNIWYFVVSLCYKFLSYFRASSTMRYSKL

SEQ ID NO: 96 - protein, 345 aa, synthetic construct
MAPNSIQNDEGLGVEVQPESLTPPKFSNIKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFAWLGWAYDLKTVSNEIVRARAMRTGDGKDLWGWNDKDITDMDKEHTDILYSKTED

SEQ ID NO: 97 - DNA, 1038 bp, synthetic construct
  source 1..1038: mol_type = other DNA
atggctcccaactctatccagaacgacgaaggcctgggcgtcgaggtgcagcccgagtctctgacccctcccaagttctctaacattaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgcctggctcggctgggcctacgacctcaagaccgtgtctaacgagatcgtgcgagcccgagccatgcgaaccggcgacggcaaggacctgtggggctggaacgacaaggacatcaccgacatggacaaggaacacactgacatcctgtactctaagaccgaggactag

SEQ ID NO: 98 - protein, 338 aa, synthetic construct
MAQTIQTTTILEQKEEKTVTLLVPKFSNIKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFAWIGWAYDLKTVSKEMIKQRSKRTGDGTNLWGLEDKDTPENLKNIKGE

SEQ ID NO: 99 - DNA, 1017 bp, synthetic construct
  source 1..1017: mol_type = other DNA
atggctcagaccatccagaccaccaccatcctcgagcagaaggaggagaagaccgtgaccctgctggtccccaagttctctaacattaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgcctggatcggatgggcctacgacctcaagacggtctccaaggagatgatcaagcagcgatccaagcgaactggcgacggaactaacctgtggggcctggaggacaaggatactcctgagaacctgaagaacatcaagggcgagtaa

SEQ ID NO: 100 - protein, 344 aa, synthetic construct
MVPNKGSSVVLTENAEPQFKKLVAPKFSNIKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFANIGWAYDLKTVPQEAVEKRMARTGDGTDLWGYGEKKPVKDVKDNSDETYYDF

SEQ ID NO: 101 - DNA, 1035 bp, synthetic construct
  source 1..1035: mol_type = other DNA
atggtgcccaacaagggatcttctgtggtgctgaccgagaacgctgagccccagttcaagaagctggtcgctcccaagttctctaacattaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgccaacatcggctgggcctacgacctcaagaccgtgcctcaagaggccgtcgagaagcgaatggcccgaaccggcgacggcaccgacctgtggggctacggcgaaaagaagcccgtcaaggacgttaaggacaactctgacgagacttactacgacttttag

SEQ ID NO: 102 - protein, 339 aa, synthetic construct
MAPNTKTSESLLKTPEDLKSSEDTKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFAWIGWAYDLKTAPDDIIRKRVERTGDGTNMWGWGDKDMSKDDRESVKILGEKFL

SEQ ID NO: 103 - DNA, 1020 bp, synthetic construct
  source 1..1020: mol_type = other DNA
atggctcccaacaccaagacctctgagtctctgctcaagacccctgaggacctgaagtcctctgaggacaccaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgcctggatcggctgggcctacgacctcaagaccgctcctgacgacatcatccgaaagcgagtcgagcgaaccggcgacggcaccaacatgtggggctggggagacaaggacatgtctaaggacgaccgagagtctgtgaagatcctgggcgagaagttcctgtaa

SEQ ID NO: 104 - protein, 326 aa, synthetic construct
MVPNKGSSDVLSEHSEPQFTKLIAPKFSNIKPKILHVNLIYFLYWHLAAPYGLYLCCTTAKWATIIFGFVMFLVAELGITAGAHRLWSHKSYKAKLPLQIILMLFNSIAFQNSIIHWARDHRLHHKYSDTEADPYNSSRGFFYSHFGWLITEKNKEVLNRAKSIDASDLYNNPVLQFQKKYAVPVFASLCFILPTLIPMYLWGESFNNSWHVNLLRYIMNLNVTLLVNSAAHRWGYKPYEKNINPTQNVYVSFVTLGEGFHNYHHIFPWDYKTAELGNNKLNVTTWFINFFAWIGWAYDLKSVPQEAIAKRCAKTGDGTDMWGRKR

SEQ ID NO: 105 - DNA, 981 bp, synthetic construct
  source 1..981: mol_type = other DNA
atggtgcccaacaagggttcttccgacgtcctgtctgagcactccgagccccagttcaccaagctgattgctcccaagttctctaacattaagcccaagatcctgcacgtgaacctgatctacttcctgtactggcacctggctgctccctacggcctgtacctgtgctgcaccaccgccaagtgggccaccatcatcttcggcttcgtgatgttcctggtggccgagctgggcatcaccgctggcgcccaccgactgtggtcccacaagtcttacaaggccaagctgcccctgcagatcatcctgatgctgttcaactctatcgccttccagaactctatcatccactgggcccgagatcaccgactgcaccacaagtactctgacaccgaggctgacccctacaactcttctcgaggcttcttctactctcacttcggctggctgatcaccgagaagaacaaggaagtgctgaaccgagccaagtctatcgacgcctctgacctgtacaacaaccccgtcctgcagttccaaaagaagtacgctgtccccgttttcgcctctctgtgcttcattctgcccactctgatccccatgtacctctggggcgagtctttcaacaactcttggcacgtcaacctgctgcggtacatcatgaacctgaacgtgaccctgctggtgaactctgccgctcaccgatggggctacaagccctacgaaaagaacatcaaccccactcagaacgtgtacgtgtctttcgtgaccctcggcgagggcttccacaactaccaccacatctttccatgggactacaagaccgctgagctgggaaacaacaagctgaacgtcactacctggttcatcaactttttcgcctggatcggatgggcttacgacctgaagtccgtcccccaggaagccatcgctaagcgatgcgctaagaccggcgacggaaccgacatgtggggacgaaagcgatag

SEQ ID NO: 106 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

(19) United States
(12) Patent Application Publication
(10) Pub. No.: US 2026/0256139 A1
(43) Pub. Date: Sep. 3, 2026

(54) BIOPESTICIDE COMPOSITION

(71) Applicant: FMC AGRICULTURAL SOLUTIONS A/S, Harboøre (DK)

(72) Inventors: Dan LUO, Copenhagen (DK); Carina HOLKENBRINK, Copenhagen (DK); Irina BORODINA, Copenhagen (DK)

(21) Appl. No.: 18/878,734

(22) PCT Filed: Jul. 3, 2023
(86) PCT No.: PCT/EP2023/068179

§ 371 Date: Dec. 24, 2024

(30) Foreign Application Priority Data
Jul. 4, 2022 (EP) 22182882.5
Feb. 13, 2023 (EP) 23156368.5
Apr. 3, 2023 (EP) 23166261.0

(51) Int. Cl. A01N 35/04; A01N 25/22; A01P 7/04; C12N 9/02; C12N 15/52; C12P 7/04; C12P 7/24; C12P 7/62

(52) U.S. Cl. CPC A01N 35/04; A01N 25/22; A01P 7/04; C12N 9/0008; C12N 9/0071; C12N 15/52; C12P 7/04; C12P 7/24; C12P 7/62; C12Y 102/01084; C12Y 114/19001; C12Y 114/19005

(57) ABSTRACT

The present disclosure relates to a biopesticide composition comprising a target compound selected from (Z,E)-9,11-hexadecadienal, and optionally one or more compounds selected from (Z)-9-hexadecenal, (Z)-11-hexadecenal and/or hexadecanal, in combination with one or more carriers, agents, additives, adjuvants and/or excipients.

### FIELD

The present disclosure describes a biobased biopesticide compositions comprising sugarcane borer Diatraea saccharalis mating pheromone components and methods for the production of such biopesticide composition. Also described herein are engineered cells and enzymes they express as well as methods for the application of such biopesticide compositions for the control of pests.

### BACKGROUND

Integrated Pest Management (IPM) is playing an increasing role for both increasing the crop yield and for minimizing environmental impact and enabling organic food production. IPM employs alternative pest control methods, such as using pheromones for pest insect mating disruption or mass trapping, or attraction of beneficial insects etc.

Pheromones constitute a group of diverse chemical compounds that insects (like other organisms) use to communicate between individuals of the same species in various contexts, including mate attraction, alarm, trail marking and aggregation. Insect pheromones associated with long-range mate finding are already used in agriculture and forestry applications for monitoring and control of pests, as a safe and environmentally friendly alternative to pesticides. The biological production of pheromones for use in pest control is advantageous over chemical synthesis in respect to price, specificity, and environmental impact.

Pheromones and pheromone precursors can be produced by genetically engineered cell factories modified to include pathways expressing enzymes necessary for converting cellular precursor metabolites into the desired pheromones and pheromone precursors, such described in WO2021078452 and WO2021123128.

Known pheromones include fatty acyl alcohols, aldehydes and acetates having one or more double bonds at specific positions of the carbon backbone having specific Z or/and E orientation. The sugarcane borer D. saccharalis is a major pest of sugarcane in the Central and Southern America. Its major mating pheromone component is (Z,E)-9,11-hexadecadienal (Svatos, et al., 2001). The minor pheromone components are (Z)-11-hexadecenal, (Z)-9-hexadecenal, and hexadecanal (Kalinova, Kindl, Hovorka, Hoskovec, & Svatos, 2005) (Da Silva, et al., 2021).

Zhao et al (2004) relates to sex pheromones (Z)-5-dodecenol and (Z,E)-5,7-dodecadienol of the moth Dendrolimus punctatus and studies the formation of compounds produced when topically applying labelled fatty acids to the moth gland. The paper further speculates that the pathway for D. punctatus sex pheromones includes formation of (Z,E)-9,11-hexadecadienoyl-CoA intermediate compounds, but no such compound is in fact detected in any of the reported experiments, although searched for. Lienard et al (2010) described yeast cells expressing a desaturase from D. punctatus named Dpu_APSQ. The authors speculate that the desaturase Dpu_APSQ introduces a E11-desaturation into (Z)-9-hexadecenoic acid natively produced in yeast producing the double-unsaturated C16-fatty acid (Z,E)-9,11-hexadecadienoic acid. However, the present inventors have found that the Dpu_APSQ does not produce (Z,E)-9,11-hexadecadienoic acid, but another unknown double unsaturated hexadecadienoic acid eluting slightly later than the (Z,E)-9,11-hexadecadienoic acid.

Accordingly, until now a bio-based method for producing D. saccharalis pheromones has not been available and accordingly, there is a need for identifying enzymes that can carry out biosynthesis of pheromone precursor and developing recombinant strains and methods for the production of such pheromones.

### SUMMARY

Until the present disclosure no enzyme has been available for expression in a genetically modified host cell for introducing E11 double bond in (Z)-9-hexadecenoyl-CoA (Z9-16:CoA)—a functionality which is essential to the production of the major mating pheromone component of D. saccharalis. However, disclosed herein is a novel enzyme which surprisingly catalyses the E11 desaturation in a (Z)-9-hexadecenoyl-CoA substrate and in the presence of such (Z)-9-hexadecenoyl-CoA substrate produces (Z,E)-9,11-hexadecadienoyl-CoA, which is a precursor for D. saccharalis pheromone (Z,E)-9,11-hexadecadienal (Z9, E11-16:Ald). Further, the present disclosure provides a Δ9-desaturase capable of introducing a Z9 double bond in (E)-11-hexadecenoyl-CoA to provide (Z,E)-9,11-hexadecadienoyl-CoA. Accordingly, the present inventors succeeded in preparing for the first time biobased biopesticide compositions comprising D. saccharalis pheromone components formulated for the use to control pests such as D. saccharalis. Accordingly, in a first aspect provided for herein is a biopesticide composition comprising (Z,E)-9,11-hexadecadienal, and optionally one or more compounds selected from (Z)-9-hexadecenal, (Z)-11-hexadecenal and/or hexadecanal, in combination with one or more carriers, agents, additives, adjuvants and/or excipients.

In a further aspect described herein is a method for controlling a pest comprising distributing the composition described herein in a habitat for the pest and allowing the target compound to control the pest.

In a still further aspect described herein is a method for producing the biopesticide composition of this disclosure comprising
(I) culturing a genetically engineered yeast cell producing hexadecanoyl-CoA and expressing
   a) a Δ9 desaturase catalyzing the formation of a double bond in a Z configuration in position 9 in the hexadecanoyl-CoA and thereby producing a (Z)-9-hexadecenoyl-CoA;
   b) an E11 desaturase catalyzing the formation of a double bond in a E configuration in position 11 in the (Z)-9-hexadecenoyl-CoA and thereby producing a (Z,E)-9,11-hexadecandienoyl-CoA;
   c) an alcohol-forming fatty acyl-CoA reductase (FAR) converting the (Z,E)-9,11-hexadecandienoyl-CoA into (Z,E)-9,11-hexadecandien-1-ol;
(II) enzymatically or chemically converting the (Z,E)-9,11-hexadecandien-1-ol to (Z,E)-9,11-hexadecandienal; and
(III) optionally recovering and/or isolating the (Z,E)-9,11-hexadecandienal and optionally one or more precursors thereof.

In a still further aspect described herein is a genetically engineered yeast cell producing (Z,E)-9,11-hexadecadienoyl-CoA and (Z,E)-9,11-hexadecadien-1-ol, said cell producing hexadecanoyl-CoA and expressing
a. a Δ9 desaturase catalyzing the formation of a double bond in a Z configuration in position 9 in the hexadecanoyl-CoA thereby producing a (Z)-9-hexadecenoyl-CoA;
b. an E11 desaturase catalyzing the formation of a double bond in a E configuration in position 11 in the (Z)-9-hexadecenoyl-CoA thereby producing a (Z,E)-9,11-hexadecadienoyl-CoA; and
c. an alcohol-forming fatty acyl-CoA reductase (FAR) converting the (Z,E)-9,11-hexadecadienyl-CoA into (Z,E)-9,11-hexadecadien-1-ol.

In a still further aspect described herein is a cell culture comprising the genetically engineered microbial cell described herein and a growth medium.

In a still further aspect described herein is an E11 desaturase having an amino acid sequence comprised in the E11 desaturase of SEQ ID NO: 1.

### DRAWINGS AND FIGURES

FIG. 1 shows the method for producing the major and minor pheromone components of D. saccharalis—(Z,E)-9,11-hexadecadienal. The method comprises biosynthesis of (Z,E)-9,11-hexadecadien-1-ol and subsequent chemical oxidation into the corresponding aldehyde (Z,E)-9,11-hexadecadienal. Due to the side biochemical reactions, the minor components of the D. saccharalis can be produced in the same process as shown.

FIG. 2 shows GC-MS analyses of FAME extracts from Yarrowia lipolytica strains expressing D. saccharalis and D. punctatus desaturases. (A) Total ion chromatogram of FAME extracts from strains expressing Ds12389 D. saccharalis desaturase (the dashed line) were compared with extracts from the control strain transformed with an empty vector (the dotted line) and authentic standard of (Z,E)-9,11-hexadecadienoic acid methyl ester (Z9, E11-16:Me) (the solid line). (B) Total ion chromatogram of FAME extracts from strains expressing Dpu_APSQ (the dotted dash line) were compared with extracts from the control strain transformed with an empty vector (the dot line) and authentic standard of Z9, E11-16:Me (the solid line).

FIG. 3 shows mass spectra of detected products from GC-MS chromatograms. (A) the product eluted at 12.954 min from the FAME extracts of Y. lipolytica strains expressing Ds12389. (B) authentic standard of Z9, E11-16:Me.

FIG. 4 shows representative GC chromatograms of (A) extracts of strains ST12118 (cultivated in medium supplemented with Z9, E11-16:Me), expressing fatty acyl-CoA reductase HarFAR (also referred to as FAR1 herein), and Z9,E11-16:OH analytical standard. Mass spectra of (B) extract of ST12118 and (C) Z9, E11-16:OH analytical standard at retention time 13.6-13.7 min.

FIG. 5 shows representative GC chromatograms of (A) extracts of strains ST13046, ST13042, ST13043 and Z9,E11-16:Me analytical standard. Mass spectra of (B) extract of ST13046, (C) extract of ST13042, (D) extract of ST13043 and (E) Z9,E11-16:Me analytical standard at retention time 11.08 min.

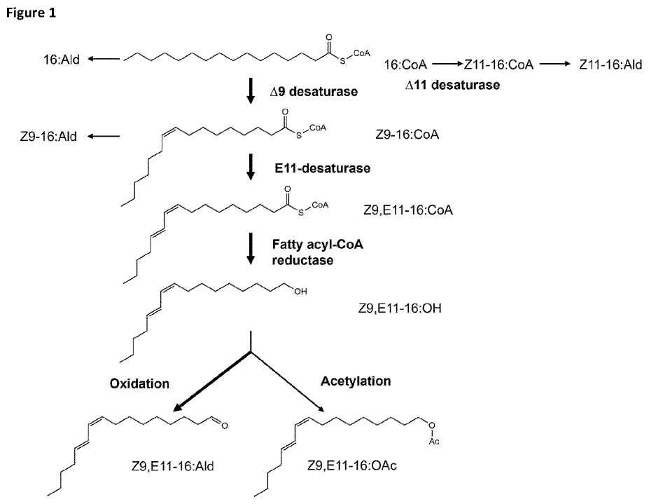

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILS

### Definitions

Throughout this disclosure where reference is made to a pheromone component or a precursor for example (Z,E)-9,11-hexadecadienal referring to the fatty aldehyde having a carbon chain of 16, having an aldehyde at C1, having a Z configured double bond at C9 and an E configured double bond at C11, alternative terms such as Z9,E11-16:Ald or (Z9, E11)-hexadecadienal may be used interchangeably. Similar nomenclature may be used about other pathway compounds such as corresponding fatty acids, CoA derivatives, alcohols, acids or acetates.

The term “saturated” refers to a compound which is devoid of double or triple carbon-carbon bonds.

The term “desaturated” as used herein interchangeably with the term “unsaturated” about compounds refers to the compound containing one or more double or triple carbon-carbon bonds, preferably a double carbon-carbon bond. The following nomenclature is used herein throughout: a Δi desaturated compound, where i is an integer, refers to a compound having a double or triple carbon-carbon bond in position i of the carbon chain. The carbon chain length is thus at least equal to i. For example, a Δ12 desaturated compound refers to a compound having a double or triple carbon-carbon bond in position 12 and having a carbon chain length of 13 or more. The double or triple bond can be in an E configuration or in a Z configuration. Thus, an Ei or a Zi desaturated compound will refer to a compound having a double carbon-carbon bond in an E configuration or in a Z configuration, respectively, in position i of the carbon chain, which has a total length at least equal to i. For example, an E11 fatty alcohol has a desaturation in position 11 in an E configuration and has a carbon chain length of 12 or more.

The term “Δ11 desaturase” as used herein refers to a desaturase enzyme which catalyzes the introduction of a double bond in position between C11 and C12 in a saturated or desaturated fatty acyl compound such as a fatty acyl coenzyme A (fatty acyl-CoA) having a carbon chain of at least 12 carbons.

The term “E11 desaturase” as used herein refers to a Δ11 desaturase enzyme which catalyzes the introduction of a double bond in a E configuration in position 11 between C11 and C12 in a saturated or desaturated fatty acyl compound such as a fatty acyl coenzyme A (fatty acyl-CoA) having a carbon chain of at least 12 carbons.

The term “Δ9 desaturase” as used herein refers to a desaturase enzyme which catalyzes the introduction of a double bond in position between C9 and C10 in a saturated or desaturated fatty acyl compound such as a fatty acyl coenzyme A (fatty acyl-CoA) having a carbon chain of at least 10 carbons.

The term “Z9 desaturase” as used herein refers to a Δ9 desaturase enzyme which catalyzes the introduction of a double bond in a Z configuration in position between C9 and C10 in a saturated or desaturated fatty acyl compound such as a fatty acyl coenzyme A (fatty acyl-CoA) having a carbon chain of at least 10 carbons.

The term “biopesticide” as used herein refers to a contraction of ‘biological pesticide’ and refers to several types of pest management interventions: through predatory, parasitic, or chemical relationships. In the EU, biopesticides have been defined as “a form of pesticide based on microorganisms or natural products”. In the US, they are defined by the EPA as “including naturally occurring substances that control pests (biochemical pesticides), microorganisms that control pests (microbial pesticides), and pesticidal substances produced by plants containing added genetic material (plant-incorporated protectants) or PIPs”. The present disclosure relates more particularly to biopesticides comprising natural products or naturally occurring substances. In the present context, these are manufactured by cultivating and concentrating naturally occurring organisms and/or their metabolites including bacteria and other microbes, fungi, nematodes, proteins, etc. These compounds are considered to be important components of integrated pest management (IPM) programs and have received much practical attention as substitutes to synthetic chemical plant protection products (PPPs). The Manual of Biocontrol Agents (2009: formerly the Biopesticide Manual) gives a review of the available biological insecticide (and other biology-based control) products.

The term “biobased” as used herein is used to characterize biobased products wherein:
(I) the total carbon content of the product is at least 30%, and
(II) the carbon content of a renewable raw material (biobased) is at least 20%.

As recognized by the Circular Bio-based Europe Joint Undertaking (CBE Joint Undertaking) established in 2021, developing biobased materials is essential if the EU is to reach its climate targets as set out in the European Green Deal. The present disclosure provides a methodology for efficiently providing fatty alcohols and fatty aldehydes having a high content of biobased carbon (%).

Both fossil and renewable raw materials consist mainly of carbon (C). Carbon occurs in several isotopes. Isotope ¹⁴C is radioactive and occurs naturally in all living organisms (plants, animals, etc) in a fixed relative concentration which is nearly identical to the relative ¹⁴C concentration in the atmosphere. At this concentration, the radioactivity level of ¹⁴C is 100%. Once an organism is no longer living, this concentration, and thus the radioactivity rate, decays with a half-life of approximately 5700 years. The radioactive ¹⁴C level of an unknown substance can therefore help determine how old the carbon contained in the substance is.

“Young” carbon (0 to 10 years) derived from renewable raw materials, such as plants or animals, has a relative isotope ¹⁴C concentration which is nearly identical to the relative ¹⁴C concentration in the atmosphere and the radioactive ¹⁴C level of such young carbon is thus about 100%.

“Old” carbon (millions of years) derived from synthetic or fossil (petrochemical) sources is greatly depleted from isotope ¹⁴C as the age of such synthetic and fossil sources far exceeds the half-life of isotope ¹⁴C which is approximately 5700 years. Hence, carbon derived from synthetic or fossil sources has a relative isotope ¹⁴C concentration around 0% and the radioactive ¹⁴C level of such old carbon is thus about 0%.

In one embodiment the term “radioactive ¹⁴C level” refer to the total radioactive ¹⁴C level of a given substance, product, or composition, as defined above.

The isotope ¹⁴C method may be used to determine the concentration of young (renewable) materials in comparison with the concentration of old (fossil) resources. The carbon content of a renewable raw material is referred to as the “biobased carbon content”. The carbon content of a renewable raw material or the “biobased carbon content” may be determined as described below.

When measuring the biobased carbon content, the result may be reported as “% biobased carbon”. This indicates the percentage carbon from “natural” (plant or animal by-product) sources versus “synthetic” or “fossil” (petrochemical) sources. For reference, 100% biobased carbon indicates that a material is entirely sourced from plants or animal by-products and 0% biobased carbon indicates that a material did not contain any carbon from plants or animal by-products. A value in between represents a mixture of natural and fossil sources.

Example: If a product has a radioactive ¹⁴C level of 80%, it means that the product consists of 80% renewable and 20% fossil carbon (C). In other words, the product is 80% biobased.

The analytical measurement may be cited as “percent modern carbon (pMC)”. This is the percentage of ¹⁴C measured in the sample relative to a modern reference standard (NIST 4990C). The % Biobased Carbon content is calculated from pMC by applying a small adjustment factor for ¹⁴C in carbon dioxide in air today. It is important to note that all internationally recognized standards using ¹⁴C assume that the plant or biomass feedstocks were obtained from natural environments. pMC may be analysed by a standard test method, such as “ASTM D6866”.

The term “fatty acyl compounds” as used herein refers to fatty compounds having a long aliphatic chain, i.e. an aliphatic chain typically having between 12 and 28 carbon atoms, such as 13, 14, 15, 16, 17, 18, 19, 20, 21, 22, 23, 24, 25, 26, 27 or 28 carbon atoms. Most naturally occurring fatty acids are unbranched. They can be saturated, or desaturated. Fatty acyl compounds can include various functional end groups.

The term “Fatty acyl-CoA” as used herein interchangeably with “fatty acyl-CoA ester” refers to compounds of general formula R—CO—SCoA, where R is a fatty carbon chain having a carbon chain length of 12 to 28 carbon atoms, such as 13, 14, 15, 16, 17, 18, 19, 20, 21, 22, 23, 24, 25, 26, 27 or 28 carbon atoms. The fatty carbon chain is joined to the —SH group of CoA by a thioester bond. Fatty acyl-CoAs can be saturated or desaturated, depending on whether the fatty acid which it is derived from is saturated or desaturated.

The term “fatty alcohol” as used herein refers to an alcohol having a carbon chain length of 13 to 28 carbon atoms, such as 13, 14, 15, 16, 17, 18, 19, 20, 21, 22, 23, 24, 25, 26, 27 or 28 carbon atoms. Fatty alcohols can be saturated or desaturated.

The term “fatty alcohol acetate” as used herein refers to an acetate having a fatty carbon chain, i.e. an aliphatic chain between 13 and 28 carbon atoms, such as 13, 14, 15, 16, 17, 18, 19, 20, 21, 22, 23, 24, 25, 26, 27 or 28 carbon atoms. Fatty acyl acetates can be saturated or desaturated.

The term “fatty aldehyde” as used herein refers to an aldehyde having a carbon chain length of 13 to 28 carbon atoms, such as 13, 14, 15, 16, 17, 18, 19, 20, 21, 22, 23, 24, 25, 26, 27 or 28 carbon atoms. Fatty aldehydes can be saturated or desaturated.

The term “functional variant” as used herein refers to functional variants of an enzyme, which retain at least some of the activity of the parent enzyme. Thus, functional variants of desaturases, or other pathway enzymes catalyze similar reactions as their parent enzymes, although the efficiency and specificity of reaction may be different, e.g. the efficiency is decreased or increased compared to the parent enzyme.

The term “heterologous” or “recombinant” or “genetically modified” and their grammatical equivalents as used herein interchangeably about nucleotides, polypeptides and cells refers to entities “derived from a different species or cell”. For example, a heterologous or recombinant polynucleotide gene is a gene in a host cell not naturally containing that gene, i.e. the gene is from a different species or cell type than the host cell. A heterologous or recombinant polypeptide is a polypeptide produced in a host cell not naturally containing the polypeptide, i.e. the polypeptide is from a different species or cell type than the host cell. Where the terms as used herein about host cells, they refer to host cells comprising and expressing heterologous or recombinant polynucleotides

The term “% identity” is used herein about the relatedness between two amino acid sequences or between two nucleotide sequences usings standard alignment software known in the art, and applying settings as instructed for the software, including gaps, to achieve the maximum percent identity/similarity/homology, and if necessary considering any conservative substitutions according to the NCIUB rules (hftp://www.chem.qmul.ac.uk/iubmb/misc/naseq.html; NC-IUB, Eur J Biochem (1985)) as part of the sequence identity. 5′ or 3′ extensions nor insertions (for nucleic acids) or N′ or C′ extensions nor insertions (for polypeptides) usually result in a reduction of identity, similarity or homology using such standard software.

The term “degeneracy” as used herein about genetic code reflects that a number of different variant nucleotide sequences can encode the same polypeptide, since there is more than one nucleotide triplet that serves as codon for a given amino acid. Thus, codons in the coding sequence for a given polypeptide can be modified such that optimal expression in a particular host is obtained, using appropriate codon bias tables for a particular host cell.

The term “pest” as used herein refers to an organism, in particular an animal such as an insect considered harmful to humans or animals or to cultivated crops, in particular within agriculture or livestock production. A pest is any living organism which is invasive or prolific, harmful, troublesome, noxious, destructive, to either crops or animals, human or human concerns, livestock, human structures, wild ecosystems etc. Particularly used herein pest is used about the sugarcane borer moth D. saccharalis and any of its preceding life stages (larvae).

The term “pheromone” as used herein is used about naturally occurring signaling compounds used in nature for chemical communication between individuals of a species. Lepidopteran pheromones for example are designated by an unbranched aliphatic chain (between 9 and 18 carbons, such as 9, 10, 11, 12, 13, 14, 15, 16, 17 or 18 carbon atoms) ending in an alcohol, aldehyde or acetate functional group and containing up to 3 double bonds in the aliphatic backbone. Thus, desaturated fatty alcohols, desaturated fatty aldehydes and desaturated fatty alcohol acetates are typically comprised in pheromones. Pheromone compositions may be produced chemically or biochemically, for example as described herein. Pheromones thus comprise desaturated fatty alcohols, desaturated fatty aldehydes and/or desaturated fatty alcohol acetates, such as can be obtained by the methods and cells described herein.

The titer of a compound as used herein refers herein to the produced concentration of a compound. When the compound is produced by a cell, the term refers to the total concentration produced by the cell, i.e. the total amount of the compound divided by the volume of the culture medium. This means that, particularly for volatile compounds, the titer includes the portion of the compound which may have evaporated from the culture medium, and it is thus determined by collecting the produced compound from the fermentation broth and from potential off-gas from the fermenter.

The term “pathway” or “biosynthetic pathway” or “metabolic pathway” as used herein interchangeably refers to one or more enzymes acting in concert in a live cell to convert one or more substrate precursors into a chemical product. A pathway may include one enzyme or multiple enzymes acting in sequence or in combination. A pathway including only one enzyme may also herein be referred to as “bioconversion” in particular relevant for embodiments where a host cell is fed with a precursor or substrate exogenously to be converted by the enzyme into a desired end product. Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds (co-factor and/or co-enzymes) which may or may not be considered part of the pathway.

The term “in vivo”, as used herein refers to within a living cell or organism, including, for example animal, a plant or a microorganism.

The term “in vitro” as used herein refers to outside a living cell or organism, including, without limitation, for example, in a microwell plate, a tube, a flask, a beaker, a tank, a reactor and the like.

The term “substrate” or “precursor” as used herein refers to any compound that can be converted into a different compound. For clarity, substrates and/or precursors include both compounds generated in situ by an enzymatic reaction in a cell or exogenously provided compounds, such as exogenously provided organic molecules which the host cell can metabolize into a desired compound.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide to be expressed in the host cell. Host cell encompasses any progeny of a parent cell including those that are not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence” and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of the host cells described herein. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source; a nitrogen source; a phosphate source; vitamins; trace elements; salts; amino acids; nucleobases; and the like.

Term “endogenous” or “native” as used herein refers to a gene or a polypeptide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but are not limited to compounds of the disclosure for which the ratio of the compounds relative to other constituents with which they are associated in nature is increased or decreased. In an important embodiment the amount of compound is increased relative to other constituents with which the compound is associated in nature. In an embodiment the compound of the disclosure may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

### Δ9 Desaturases

The present disclosure further provides a Δ9 desaturase comprising an amino acid sequence which is the desaturase comprised in SEQ ID NO: 14, 15, 16, 17, 18, 19, 20, 21, 22, or 82 or is a functional variant at least 50% identical to the desaturase comprised in SEQ ID NO: 14, 15, 16, 17, 18, 19, 20, 21, 22, or 82. In some embodiments the Δ9 desaturase is from 50% to 100% identical to the Δ9 desaturase comprised in SEQ ID NO: 14, 15, 16, 17, 18, 19, 20, 21, 22, or 82, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. The Δ9 desaturase of the disclosure refers to a desaturase enzyme which catalyzes the introduction of a double bond in position between C9 and C10 in a saturated or desaturated fatty acyl compound such as a fatty acyl coenzyme A (fatty acyl-CoA) having a carbon chain of at least 10 carbon atoms.

### Δ9 Genes

A further aspect provides for a polynucleotide sequence (Δ9 desaturase gene) encoding a Δ9 desaturase said polynucleotide sequence being at least 50% identical to the Δ9 desaturase coding sequence comprised in SEQ NO:83. In some embodiments the Δ9 desaturase gene is from 50% to 100% identical to the Δ9 desaturase gene comprised in SEQ ID NO: 83, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In preferred embodiments, the Δ9 desaturase gene encodes the Δ9 desaturase of SEQ IN NO: 82 or the described functional variants. On other embodiments the Δ9 desaturase gene is codon optimized for heterologous expression in a microorganism, in particular a yeast.

### E11 Desaturases

One aspect provides for an E11 fatty acyl-CoA desaturase (E11 desaturase) comprising an amino acid sequence which is the E11 desaturase comprised in SEQ ID NO:1 or is a functional variant at least 50% identical to the E11 desaturase comprised in SEQ ID NO: 1. In some embodiments the E11 desaturase is from 50% to 100% identical to the E11 desaturase comprised in SEQ ID NO: 1, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In more specific embodiments the E11 desaturase catalyzes the introduction of a double bond in a E configuration in position 11 in a (Z9)-9-hexadecenoyl-CoA substrate a (Z,E)-9,11-hexadecadienoyl-CoA having a double bond in position 9 in Z configuration and in position 11 in E configuration.

A further aspect provides for an E11 fatty acyl-CoA desaturase (E11 desaturase) comprising an amino acid sequence which is the E11 desaturase comprised in SEQ ID NO:1 or 80 or is a functional variant at least 50% identical to the E11 desaturase comprised in SEQ ID NO: 1 or 80. In some embodiments the E11 desaturase is from 50% to 100% identical to the E11 desaturase comprised in SEQ ID NO: 1 or 80, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In more specific embodiments the E11 desaturase catalyzes the introduction of a double bond in a E configuration in position 11 in a (Z9)-9-hexadecenoyl-CoA substrate to provide (Z,E)-9,11-hexadecadienoyl-CoA having a double bond in position 9 in Z configuration and in position 11 in E configuration.

A further aspect provides for an E11 fatty acyl-CoA desaturase (E11 desaturase) comprising an amino acid sequence which is the E11 desaturase comprised in SEQ ID NO:80 or is a functional variant at least 50% identical to the E11 desaturase comprised in SEQ ID NO: 80. In some embodiments the E11 desaturase is from 50% to 100% identical to the E11 desaturase comprised in SEQ ID NO: 80, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In more specific embodiments the E11 desaturase catalyzes the introduction of a double bond in a E configuration in position 11 in a (Z9)-9-hexadecenoyl-CoA substrate to provide (Z,E)-9,11-hexadecadienoyl-CoA having a double bond in position 9 in Z configuration and in position 11 in E configuration.

In some embodiments, an E11 fatty acyl-CoA desaturase (E11 desaturase) is provided comprising an amino acid sequence having at least 50% identity to the E11 desaturase comprised in SEQ ID NO: 1, SEQ ID NO: 80, SEQ ID NO: 90, or SEQ ID NO: 92, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical.

In some embodiments, an E11 fatty acyl-CoA desaturase (E11 desaturase) is provided comprising an amino acid sequence having at least 50% identity to the E11 desaturase comprised in SEQ ID NO: 1, SEQ ID NO: 80, SEQ ID NO: 90, SEQ ID NO: 92, SEQ ID NO: 96, SEQ ID NO: 98, SEQ ID NO: 100, SEQ ID NO: 102, or SEQ ID NO: 104, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical.

### E11 Genes

A further aspect provides for a polynucleotide sequence (E11 desaturase gene) encoding an E11 desaturase said polynucleotide sequence being at least 50% identical to the E11 desaturase coding sequence comprised in SEQ NO:2. In some embodiments the E11 desaturase gene is from 50% to 100% identical to the E11 desaturase gene comprised in SEQ ID NO: 2 or 93, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In preferred embodiments the E11 desaturase gene encodes the E11 desaturase of SEQ IN NO: 1 or the described functional variants. On other embodiments the E11 desaturase gene is codon optimized for heterologous expression in a microorganism, in particular a yeast.

A further aspect provides for a polynucleotide sequence (E11 desaturase gene) encoding an E11 desaturase said polynucleotide sequence being at least 50% identical to the E11 desaturase coding sequence comprised in SEQ NO:2 or 81. In some embodiments the E11 desaturase gene is from 50% to 100% identical to the E11 desaturase gene comprised in SEQ ID NO: 2, 81 or 93, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In preferred embodiments the E11 desaturase gene encodes the E11 desaturase of SEQ IN NO: 1 or 80 or the described functional variants. On other embodiments the E11 desaturase gene is codon optimized for heterologous expression in a microorganism, in particular a yeast.

A further aspect provides for a polynucleotide sequence (E11 desaturase gene) encoding an E11 desaturase said polynucleotide sequence being at least 50% identical to the E11 desaturase coding sequence comprised in SEQ NO:81. In some embodiments the E11 desaturase gene is from 50% to 100% identical to the E11 desaturase gene comprised in SEQ ID NO: 81, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical. In preferred embodiments the E11 desaturase gene encodes the E11 desaturase of SEQ IN NO: 80 or the described functional variants. On other embodiments the E11 desaturase gene is codon optimized for heterologous expression in a microorganism, in particular a yeast.

In some embodiments, a polynucleotide sequence codon optimized for heterologous expression encoding the E11 desaturase disclosed herein is provided having a DNA sequence comprised in SEQ NO:2, SEQ ID NO: 81, SEQ ID NO: 91, or SEQ ID NO: 93 or a homologue thereof including variations due to the degeneracy of the genetic code. In some embodiments, the E11 desaturase gene is from 50% to 100% identical to the E11 desaturase gene comprised in SEQ NO:2, SEQ ID NO: 81, SEQ ID NO: 91, or SEQ ID NO: 93, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical.

In some embodiments, a polynucleotide sequence codon optimized for heterologous expression encoding the E11 desaturase disclosed herein is provided having a DNA sequence comprised in SEQ NO:2, SEQ ID NO: 81, SEQ ID NO: 91, or SEQ ID NO: 93 or a homologue thereof including variations due to the degeneracy of the genetic code. In some embodiments, the E11 desaturase gene is from 50% to 100% identical to the E11 desaturase gene comprised in SEQ NO:2, SEQ ID NO: 81, SEQ ID NO: 91, SEQ ID NO: 93, SEQ ID NO: 97, SEQ ID NO: 99, SEQ ID NO: 101, SEQ ID NO: 103, or SEQ ID NO: 105, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical.

### Z11 Desaturases

In some embodiments, the present disclosure provides a Z11 desaturase having at least 70% sequence identity to an amino acid sequence comprised in the Z11 desaturase of SEQ ID NO: 72, 74, 76, or 78. In some embodiments, the Z11 desaturase is expressed in the genetically engineered host cell of the present disclosure. In some embodiments, the Z11 desaturase having at least 70% sequence identity to an amino acid sequence comprised in the Z11 desaturase of SEQ ID NO: 72, 74, 76, or 78 is employed in a method disclosed herein. In some embodiments, the Z11 desaturase has from 70% to 100% sequence identity to an amino acid sequence comprised in the Z11 desaturase of SEQ ID NO: 72, 74, 76, or 78, such as from 70% to 80%, such as from 80% to 90%, such as from 90% to 92%, such as from 92% to 94%, such as from 94% to 96%, such as from 96% to 98%, such as from 98% to 99%, such 100% sequence identity.

### Gene Constructs

A further aspect provides for a polynucleotide construct comprising an E11 desaturase gene as described operably linked to one or more control sequences. Such control sequence may be native or heterologous to the E11 desaturase gene. The polynucleotide construct can further be integrated in an expression vector for expression of the E11 desaturase gene in a host cell.

### Genetically Engineered Host Cells

A further aspect provides for a genetically engineered microbial cell producing a (Z,E)-9,11-hexadecadienoyl-CoA said cell heterologously expressing the E11 desaturase of this disclosure, which in the presence of a (Z)-9-hexadecenoyl-CoA substrate introduces a double bond in a E configuration in position 11 in the (Z)-9-hexadecenoyl-CoA substrate and thereby produces the (Z,E)-9,11-hexadecadienoyl-CoA having a double bond in position 11 in E configuration. Further, the present disclosure provides a genetically engineered microbial cell as defined herein expressing a Δ9-desaturase capable of introducing a Z9 double bond in (E)-11-hexadecenoyl-CoA to provide (Z,E)-9,11-hexadecadienoyl-CoA.

The cell provided herein can further comprise an operative biosynthetic pathway converting the (Z,E)-9,11-hexadecadienoyl-CoA into a target compound selected from
a) (Z,E)-9,11-hexadecadien-1-ol;
b) (Z,E)-9,11-hexadecadienal; and/or
c) (Z,E)-9,11-hexadecadienyl acetate;
   said pathway expressing one or more pathway polypeptides selected from:
a) an alcohol-forming fatty acyl-CoA reductase (FAR) converting (Z,E)-9,11-hexadecadienoyl-CoA into (Z,E)-9,11-hexadecadien-1-ol;
b) an acetyltransferase converting (Z,E)-9,11-hexadecadien-1-ol into (Z,E)-9,11-hexadecadienyl acetate;
c) an alcohol dehydrogenase converting (Z,E)-9,11-hexadecadien-1-ol into (Z,E)-9,11-hexadecadienal; and/or
d) a fatty alcohol oxidase converting (Z,E)-9,11-hexadecadien-1-ol into (Z,E)-9,11-hexadecadienal.

In some embodiments, the cell provided herein further comprises an operative biosynthetic pathway converting the (Z,E)-9,11-hexadecadienoyl-CoA into a target compound selected from
a) (Z,E)-9,11-hexadecadien-1-ol;
b) (Z,E)-9,11-hexadecadienal; and/or
c) (Z,E)-9,11-hexadecadienyl acetate;
   said pathway expressing one or more pathway polypeptides selected from:
d) an alcohol-forming fatty acyl-CoA reductase (FAR) converting (Z,E)-9,11-hexadecadienoyl-CoA into (Z,E)-9,11-hexadecadien-1-ol;
e) an acetyltransferase converting (Z,E)-9,11-hexadecadien-1-ol into (Z,E)-9,11-hexadecadienyl acetate;
f) a fatty alcohol oxidase converting (Z,E)-9,11-hexadecadien-1-ol into (Z,E)-9,11-hexadecadienal.

In some embodiments, the cell is provided wherein:
a) the FAR is at least 70% identical to the FAR comprised in SEQ ID NO: 46, 47, 48, 49, 50, 51, 52, 53, 54, 55, 56, 57, 58, 60, 61, 62, 63, 64, 66, 88, or 95;
b) the acetyltransferase is at least 70% identical to the acetyltransferase comprised in SEQ ID NO: 106;
c) the fatty alcohol oxidase is at least 70% identical to the fatty alcohol oxidase comprised in SEQ ID NO: 70.

Where the cell expresses such pathway enzymes the
(I) FAR is preferably at least 70% identical, such as at least 80%, such as at least 90%, such as at least 95%, such as 100% identical to the FAR comprised in SEQ ID NO: 45, 46, 47, 48, 49, 50, 51, 52, 53, 54, 55, 56, 57, 58, 59, 60, 61, 62, 63, 64, or 65;
(II) the acetyltransferase is preferably at least 70% identical, such as at least 80%, such as at least 90%, such as at least 95%, such as 100% identical to the acetyltransferase comprised in SEQ ID NO: 71;
(III) the alcohol dehydrogenase is preferably at least 70% identical, such as at least 80%, such as at least 90%, such as at least 95%, such as 100% identical to the alcohol dehydrogenase comprised in SEQ ID NO: 68; and
(IV) the fatty alcohol oxidase is preferably at least 70% identical, such as at least 80%, such as at least 90%, such as at least 95%, such as 100% identical to the fatty alcohol oxidase comprised in SEQ ID NO: 69 or 70.

In some embodiments,
(I) the FAR is at least 70% identical to the FAR comprised in SEQ ID NO: 46, 47, 48, 49, 50, 51, 52, 53, 54, 55, 56, 57, 58, 60, 61, 62, 63, 64, 66, 88, or 95;
(II) the acetyltransferase is at least 70% identical to the acetyltransferase comprised in SEQ ID NO: 71;
(III) the alcohol dehydrogenase is at least 70% identical to the alcohol dehydrogenase comprised in SEQ ID NO: 68; and
(IV) the fatty alcohol oxidase is at least 70% identical to the fatty alcohol oxidase comprised in SEQ ID NO: 69 or 70.

The genetically modified host cell can further comprise an operative biosynthetic pathway producing the (Z)-9-hexadecenoyl-CoA substrate, in particular a pathway expressing one or more heterologous Δ9 desaturases introducing a double bond in the hexadecenoyl-CoA substrate in a Z configuration in position 9. Such Δ9 desaturases are suitably at least 70% identical, such as at least 80%, such as at least 90%, such as at least 95%, such as 100% identical to the Δ9 desaturase comprised in SEQ ID NO: 14, 15, 16, 17, 18, 19, 20, 21, 22, or 82.

The genetically modified host cell can further comprise an operative biosynthetic pathway producing the (Z)-9-hexadecenoyl-CoA substrate, in particular a pathway expressing one or more heterologous Δ9 desaturases introducing a double bond in the hexadecenoyl-CoA substrate in a Z configuration in position 9. Such Δ9 desaturases are suitably at least 70% identical, such as at least 80%, such as at least 90%, such as at least 95%, such as 100% identical to the Δ9 desaturase comprised in SEQ ID NO: 14, 15, 16, 17, 18, 19, 20, 21, or 22.

In further embodiments the host cell is further modified so that one or more native or endogenous genes are attenuated, disrupted and/or deleted. Other modifications include overexpressing one or more pathway genes or modifications providing an increased amount of a substrate for at least one enzyme of the pathways described herein. The genetically modified host cell can also be further modified to exhibit increased tolerance towards one or more substrates, intermediates, or product molecules from the pathways described herein and/or the host cell can comprise at least two copies of one or more genes in such pathways. In some embodiments, the host cell can comprise at least two copies of one or more genes in the (Z,E)-9,11-hexadecadien-1-ol pathway.

The host cells provided for herein are suitably fungal cells such as yeast cells. Preferred yeasts include those belonging to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.

However, also filamentous fungal cell can be useful for example those selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

In alternative or additional aspects provided for herein is a genetically engineered yeast cell producing (Z,E)-9,11-hexadecadienoyl-CoA and (Z,E)-9,11-hexadecadien-1-ol, said cell producing hexadecanoyl-CoA and expressing
a) a Δ9 desaturase catalyzing the formation of a double bond in a Z configuration (Z) in position 9 in the hexadecanoyl-CoA thereby producing a (Z)-9-hexadecenoyl-CoA;
b) an E11 desaturase catalyzing the formation of a double bond in a E configuration in position 11 in the (Z)-9-hexadecenoyl-CoA thereby producing a (Z,E)-9,11-hexadecadienoyl-CoA; and
c) an alcohol-forming fatty acyl-CoA reductase (FAR) converting the (Z,E)-9,11-hexadecadienoyl-CoA into (Z,E)-9,11-hexadecadien-1-ol.

The yeast cell may in further embodiments express one or more enzymes selected from:
a) a Z11 desaturase catalyzing the formation of a double bond in a Z configuration in position 11 in the hexadecanoyl-CoA thereby producing a (Z)-11-hexadecenoyl-CoA;
b) one or more alcohol-forming fatty acyl-CoA reductases (FAR) converting respectively the hexadecanoyl-CoA into hexadecane-1-ol, the (Z)-9-hexadecenoyl-CoA into (Z)-9-hexadecen-1-ol, and the (Z)-11-hexadecenoyl-CoA into (Z)-11-hexadecen-1-ol.

In some embodiments, a genetically engineered yeast cell is provided producing (Z,E)-9,11-hexadecadienoyl-CoA and (Z,E)-9,11-hexadecadien-1-ol, said yeast cell producing hexadecanoyl-CoA and expressing
(I) an E11 desaturase catalyzing the formation of a double bond in a E configuration in position 11 in the hexadecanoyl-CoA and thereby producing an E-11-hexadecenoyl-CoA;
(II) a Δ9 desaturase catalyzing the formation of a double bond in a Z configuration in position 9 in the (E)-11-hexadecenoyl-CoA and thereby producing a (Z,E)-9,11-hexadecadienoyl-CoA;
(III) an alcohol-forming fatty acyl-CoA reductase (FAR) converting the (Z,E)-9,11-hexadecadienoyl-CoA into (Z,E)-9,11-hexadecadien-1-ol.

As demonstrated in the examples herein, including Example 18, the cells of the present disclosure can produce (Z,E)-9,11-hexadecadienoyl-CoA and (Z,E)-9,11-hexadecadien-1-ol through any order of desaturation i.e. by either first introducing the E-configured double bond at position 11 and then the Z-configured double bond at position 9, or vice versa.

In still further embodiments the
a) E11 desaturase has an amino acid sequence which is at least 50% identical to the E11 desaturase comprised in SEQ ID NO: 1, such as from 50% to 100%, such as from 50% to 60%, such as from 60% to 70%, such as from 70% to 80%, such as from 80% to 90%, such as from 90% to 92%, such as from 92% to 94%, such as from 94% to 96%, such as from 96% to 98%, such as from 98% to 99%, such 100% identical to the E11 desaturase comprised in the E11 desaturase of SEQ ID NO: 1;
b) Δ9 desaturase has an amino acid sequence which is at least 70% identical, such as at least 80%, such as at least 90%, such as at least 95%, such as 100% identical to the Δ9 desaturase comprised in SEQ ID NO: 14, 15, 16, 17, 18, 19, 20, 21, 22, or 82;
c) Z11 desaturase has an amino acid sequence which is at least 70% identical, such as at least 80%, such as at least 90%, such as at least 95%, such as 100% identical to the Z11 desaturase comprised in SEQ ID NO: 72, 74, 76 or 78; and
d) alcohol-forming fatty acyl-CoA reductases (FAR) has an amino acid sequence which is at least 70% identical, such as at least 80%, such as at least 90%, such as at least 95%, such as 100% identical to the FAR comprised in SEQ ID NO: 45, 46, 47, 48, 49, 50, 51, 52, 53, 54, 55, 56, 57, 58, 59, 60, 61, 62, 63, 64, or 65.

In this aspect the yeast cell is preferably of the species Saccharomyces cerevisiae, or Yarrowia lipolytica.

In some embodiments, the yeast cell is provided wherein the
(I) E11 desaturase has at least 70% sequence identity to an amino acid sequence comprised in the E11 desaturase of SEQ ID NO: 1, 80, 90, 92, 96, 98, 100, 102, or 104;
(II) Δ9 desaturase has at least 70% sequence identity to an amino acid sequence comprised in the Δ9 desaturase of SEQ ID NO: 14, 15, 16, 17, 18, 19, 20, 21, 22, or 82;
(III) Z11 desaturase has at least 70% sequence identity to an amino acid sequence comprised in the Z11 desaturase of SEQ ID NO: 72, 74, 76, or 78; and/or
(IV) alcohol-forming fatty acyl-CoA reductases (FAR) has at least 70% sequence identity to an amino acid sequence comprised in the FAR of SEQ ID NO: 46, 47, 48, 49, 50, 51, 52, 53, 54, 55, 56, 57, 58, 59, 60, 61, 62, 63, 64, 65, 66, 88, or 95.

In some embodiments, the yeast cell is provided wherein the
(I) E11 desaturase has an amino acid sequence comprised in the E11 desaturase of SEQ ID NO: 1, 80, 90, 92, 96, 98, 100, 102, or 104;
(II) Δ9 desaturase has an amino acid sequence comprised in the Δ9 desaturase of SEQ ID NO: 14, 15, 16, 17, 18, 19, 20, 21, 22, or 82;
(III) Z11 desaturase has an amino acid sequence comprised in the Z11 desaturase of SEQ ID NO: 72, 74, 76, or 78; and/or
(IV) alcohol-forming fatty acyl-CoA reductases (FAR) has an amino acid sequence comprised in the FAR of SEQ ID NO: 46, 47, 48, 49, 50, 51, 52, 53, 54, 55, 56, 57, 58, 59, 60, 61, 62, 63, 64, 65, 66, 88, or 95.

### Cultures

A further aspect provides for a cell culture, comprising host cells described herein and a growth medium. Suitable growth mediums for eukaryotic and prokaryotic cell are well known in the art.

### Methods of Producing Compounds of the Disclosure.

A further aspect provides for a method for producing a target compound selected from:
(I) (Z,E)-9,11-hexadecadienoyl-CoA;
(II) (Z,E)-9,11-hexadecadien-1-ol;
(III) (Z,E)-9,11-hexadecadienal; and/or
(IV) (Z,E)-9,11-hexadecadienyl acetate;
   said method comprising culturing the cell culture described herein at conditions allowing the cell culture to produce the target compound; and optionally recovering and/or isolating the target compound.

The cell culture can be cultivated in a nutrient medium using methods known in the art at conditions suitable for production of the target compound and/or its precursors and/or for propagating cell count. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium comprises a carbon source (e.g. glucose, maltose, molasses, starch, cellulose, xylan, pectin, lignocellolytic biomass hydrolysate, etc.), a nitrogen source (e. g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

Culturing of the host cell may be performed over a period of about 0.5 to about 30 days. The cultivation process may be a batch process, continuous or fed-batch process, suitably performed at a temperature in the range of 0-100° C. or 10-80° C., for example, from about 20° C. to about 50° C. and/or at a pH, for example, from about 2 to about 10. Preferred fermentation conditions for yeast and filamentous fungi are a temperature in the range of from about 25° C. to about 55° C. and at a pH of from about 3 to about 9. The appropriate conditions are usually selected based on the choice of host cell. Accordingly, in an embodiment the method of the disclosure further comprises one or more elements selected from:
(I) culturing the cell culture in a nutrient medium;
(II) culturing the cell culture under aerobic or anaerobic conditions
(III) culturing the cell culture under agitation;
(IV) culturing the cell culture at a temperature of between 25 to 50° C.;
(V) culturing the cell culture at a pH of between 3-9; and
(VI) culturing the cell culture for between 10 hours to 30 days.

The cell culture of the disclosure may be recovered and or isolated using methods known in the art. For example, the target compound may be recovered from the nutrient medium by conventional procedures including, but not limited to, centrifugation, filtration, spray-drying, or lyophilization. In a particular embodiment the method includes a recovery and/or isolation step comprising separating a liquid phase of the cell or cell culture from a solid phase of the cell or cell culture to obtain a supernatant comprising the target compound and/or subjecting the supernatant to one or more steps selected from:
(I) disrupting the cells of the cell culture to release intracellular target compound into the supernatant;
(II) separating the supernatant from the solid phase of the cell culture, such as by filtration or gravity separation;
(III) contacting the supernatant with one or more adsorbent resins to obtain at least a portion of the produced target compound;
(IV) contacting the supernatant with one or more ion exchange or reversed-phase chromatography columns in order to obtain at least a portion of the target compound;
(V) extracting the target compound; and/or
(VI) precipitating the E11 fatty acyl compound by crystallization or evaporating the solvent of the liquid phase; and optionally isolating the target compound by filtration or gravity separation;
   thereby recovering and/or isolating the target compound.

The method described herein may comprise one or more in vitro steps in the process of producing the target compound. Accordingly, in one embodiment the method further comprises feeding the cell culture with one or more precursor or substrates in the pathway of the target compound. Where the target compound is not the desired end products further steps may be added to the method of the disclosure either chemically or biologically/enzymatically modifying the target compound, such as oxidation and/or acetylation. Accordingly in attractive embodiments the in vitro performed step comprises chemically or enzymatically reducing (Z,E)-9,11-hexadecadienoyl-CoA into (Z,E)-9,11-hexadecadien-1-ol (Z9, E11-16:OH), preferably using a reductase enzyme (FAR). In other attractive embodiments the in vitro performed step comprises chemically or enzymatically reducing a (Z,E)-9,11-hexadecadienoic acid into (Z,E)-9,11-hexadecadien-1-ol. In further attractive embodiments the in vitro performed step comprises chemical or enzymatic oxidation of (Z,E)-9,11-hexadecadien-1-ol into (Z,E)-9,11-hexadecadienal. In still further attractive embodiments the in vitro performed step comprises chemical or enzymatic acetylation of (Z,E)-9,11-hexadecadien-1-ol into (Z,E)-9,11-hexadecadienyl acetate.

The method can further comprise recovering the target compound and mixing it with one or more carriers, agents, additives, adjuvants and/or excipients to produce a biopesticide composition.

In the method the one or more carriers, agents, additives, adjuvants and/or excipients preferably comprises a protective agent preferably comprises a conjugated sulfur, which protects the target compound from being converted into an acid. Such protective agents suitably include compounds selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione shown to have a remarkably high effect in stabilizing fatty aldehydes from oxidation. In some embodiments the method further comprises mixing in at least 10 mg of the protective agent per gram aldehyde and/or alcohol. The one or more carriers, agents, additives, adjuvants and/or excipients may also comprise a carrier facilitating slow release of the target compound, optionally being (i) a polymeric substrate selected from plastic, wax emulsion, oil emulsion, or microcapsules and/or (ii) zeolite.

In preferred embodiments target compound is a (Z,E)-9,11-hexadecadien-1-ol, a (Z,E)-9,11-hexadecadienal or a (Z,E)-9,11-hexadecadienyl acetate (Z9, E11-16:OAc). In an alternative aspect a method is provided for producing a biopesticide composition described herein comprising
(II) culturing a genetically engineered yeast cell producing hexadecanoyl-CoA and expressing
   a) a Δ9 desaturase catalyzing the formation of a double bond in a Z configuration in position 9 in the hexadecanoyl-CoA and thereby producing a (Z)-9-hexadecenoyl-CoA;
   b) an E11 desaturase catalyzing the formation of a double bond in a E configuration in position 11 in the (Z)-9-hexadecenoyl-CoA and thereby producing a (Z,E)-9,11-hexadecadienoyl-CoA;
   c) an alcohol-forming fatty acyl-CoA reductase (FAR) converting the (Z,E)-9,11-hexadecadienoyl-CoA into (Z,E)-9,11-hexadecadien-1-ol;
(IV) enzymatically or chemically converting the (Z,E)-9,11-hexadecadien-1-ol to (Z,E)-9,11-hexadecadienal; and
(V) optionally recovering and/or isolating the (Z,E)-9,11-hexadecadienal and optionally one or more precursors thereof.

In some embodiments in this alternative aspect the genetically engineered yeast cell further expresses one or more enzymes selected from:
a) a Z11 desaturase catalyzing the formation of a double bond in a Z configuration (Z) in position 11 in the hexadecanoyl-CoA and thereby producing a (Z)-11-hexadecenoyl-CoA;
b) one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol;
   and the method further comprises enzymatically or chemically converting the hexadecane-1-ol into hexadecanal, the (Z)-9-hexadecen-1-ol into (Z)-9-hexadecenal and the (Z)-11-hexadecen-1-ol and the (Z)-11-hexadecen-1-ol into (Z)-11-hexadecenal; and optionally recovering and/or isolating the hexadecanal, the (Z)-9-hexadecenal and the (Z)-11-hexadecenal and optionally one or more precursors thereof.

In some embodiments, an alternative method for producing a biopesticide composition is provided comprising the steps:
(I) culturing a genetically engineered yeast cell producing hexadecanoyl-CoA and expressing
   a. an E11 desaturase catalyzing the formation of a double bond in a E configuration in position 11 in the hexadecanoyl-CoA and thereby producing an E-11-hexadecenoyl-CoA;
   b. a Δ9 desaturase catalyzing the formation of a double bond in a Z configuration in position 9 in the (E)-11-hexadecenoyl-CoA and thereby producing a (Z,E)-9,11-hexadecadienoyl-CoA;
   c. an alcohol-forming fatty acyl-CoA reductase (FAR) converting the (Z,E)-9,11-hexadecadienoyl-CoA into (Z,E)-9,11-hexadecadien-1-ol;
(II) enzymatically or chemically converting the (Z,E)-9,11-hexadecadien-1-ol to (Z,E)-9,11-hexadecadienal; and
(III) optionally recovering and/or isolating the (Z,E)-9,11-hexadecadienal and optionally one or more precursors thereof.

The successful use of this alternative method is demonstrated in Example 18, showcasing the possibility of introducing the E11 double bond prior to the Z9 double bond in the hexadecanoyl-CoA.

In some embodiments, the method is provided wherein the genetically engineered yeast cell further expresses one or more enzymes selected from:
a. a Z11 desaturase catalyzing the formation of a double bond in a Z configuration in position 11 in the hexadecanoyl-CoA and thereby producing a (Z)-11-hexadecenoyl-CoA;
b. one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol;
   and the method further comprises enzymatically or chemically converting the hexadecan-1-ol into hexadecanal, the (Z)-9-hexadecen-1-ol into (Z)-9-hexadecenal and the (Z)-11-hexadecen-1-ol and the (Z)-11-hexadecen-1-ol into (Z)-11-hexadecenal; and/or optionally recovering and/or isolating the hexadecanal, the (Z)-9-hexadecenal and the (Z)-11-hexadecenal and optionally one or more precursors thereof.

In other embodiments in this alternative aspect the method further comprises mixing the recovered (Z,E)-9,11-hexadecadienal, (Z)-9-hexadecenal, (Z)-11-hexadecenal, hexadecanal and optionally one or more precursors thereof with one or more carriers, agents, additives, adjuvants and/or excipients to produce the biopesticide composition. These carriers, agents, additives, adjuvants and/or excipients preferably comprise one or more compounds selected from
a) a protective agent comprising a conjugated sulfur compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione, which protects the target compound from being converted into an acid; and/or
b) a carrier facilitating slow release of the (Z,E)-9,11-hexadecadienal, (Z)-9-hexadecenal, (Z)-11-hexadecenal and/or hexadecanal from the mixture, optionally being (i) a polymeric substrate selected from plastic, wax emulsion, oil emulsion, or microcapsules and/or (ii) a zeolite.

### Composition

A further aspect provides for a biopesticide composition comprising a target compound selected from:
a) (Z,E)-9,11-hexadecadien-1-ol (Z9, E11-16:OH);
b) (Z,E)-9,11-hexadecadienal (Z9, E11-16: Ald); and/or
c) (Z,E)-9,11-hexadecadienyl acetate (Z9, E11-16:OAc); and
   one or more carriers, agents, additives, adjuvants and/or excipients.

A further alternative aspect provides for a biopesticide composition comprising a target compound selected from (Z,E)-9,11-hexadecadienal, and optionally one or more compounds selected from (Z)-9-hexadecenal, (Z)-11-hexadecenal and/or hexadecanal, in combination with one or more carriers, agents, additives, adjuvants and/or excipients. The biopesticide composition further comprises in certain embodiments at least trace amounts of one or more compounds selected from hexadecan-1-ol, (Z)-9-hexadecen-1-ol, (Z)-11-hexadecen-1-ol, and (Z,E)-9,11-hexadecadien-1-ol, other metabolites of the cell culture. In other embodiments the biopesticide composition further comprises (Z,E)-9,11-hexadecandienyl acetate. In preferred embodiments, in the biopesticide composition at least the one or more of the hexadecan-1-ol, (Z)-9-hexadecen-1-ol, (Z)-11-hexadecen-1-ol, and (Z,E)-9,11-hexadecadien-1-ol are obtained from culturering genetically engineered host cell described herein and optionally the composition comprises one or more further compounds or metabolites from the cell culture. Such compounds and/or metabolites of the cell culture includes precursors for the target compound as well as compounds selected from trace metals, vitamins, salts, yeast nitrogen base, carbon source, YNB, and/or amino acids of the fermentation. In particular the biopesticide composition comprises a concentration of the target compound of at least 1 mg/kg composition, such as at least 5 mg/kg, such as at least 10 mg/kg, such as at least 20 mg/kg, such as at least 50 mg/kg, such as at least 100 mg/kg, such as at least 500 mg/kg, such as at least 1,000 mg/kg, such as at least 5,000 mg/kg, such as at least 10,000 mg/kg, such as at least 50,000 mg/kg.

The composition can also advantageously include one or more a protective agents comprising a conjugated sulfur, which protects the target compound from from being further converted into an acid. Such protective agents comprise compounds selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione. The composition preferably comprises at least 10 mg of the protective agent per gram of target compound.

The composition further comprises in some embodiments a carrier facilitating slow release of the target compound, optionally being (i) a polymeric substrate selected from plastic, wax emulsion, oil emulsion, or microcapsules and/or (ii) zeolite.

In some embodiments, the biopesticide composition comprises at least 50% biobased carbon, such as at least 55%, such as at least 60%, such as at least 65%, such as at least 70%, such as at least 75%, such as at least 80%, such as at least 85%, such as at least 90%, such as at least 95%, such as at least 99%, such as at least 100%.

In some embodiments, the biopesticide composition comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

In some embodiments, the biopesticide composition comprises from 20% to 100% biobased carbon, such as from 30% to 100% biobased carbon, such as from 40% to 100% biobased carbon, such as from 50% to 100% biobased carbon, such as from 60% to 100% biobased carbon, such as from 70% to 100% biobased carbon, such as from 75% to 100% biobased carbon, such as from 80% to 100% biobased carbon, such as from 85% to 100% biobased carbon, such as from 90% to 100% biobased carbon, such as from 95% to 100% biobased carbon, such as 100% biobased carbon.

In some embodiments, the biopesticide composition comprises no more than 50% fossil-based carbon, such as no more than 45%, such as no more than 40%, such as no more than 35%, such as no more than 30%, such as no more than 25%, such as no more than 20%, such as no more than 15%, such as no more than 10%, such as no more than 5%, such as no more than 1% fossil-based carbon.

In some embodiments, the biopesticide composition comprises 90% biobased carbon, 91% biobased carbon, 92% biobased carbon, 93% biobased carbon, 94% biobased carbon, 95% biobased carbon, 96% biobased carbon, 97% biobased carbon, 98% biobased carbon, 99% biobased carbon, or 100% biobased carbon, for example 94% biobased carbon.

In some embodiments, the biopesticide composition comprises Z9,E11-16:OH which is at least 90% biobased, such as at least 92% biobased carbon, such as at least 94% biobased carbon, such as at least 96% biobased carbon, such as at least 98% biobased carbon, such as 100% biobased carbon, for example 94% biobased.

### Applications

A further aspect provides for a method of controlling or monitoring a pest comprising distributing the composition described herein in a habitat for the pest and allowing the target compound to control the pest. The target compounds described herein are particularly effective again D. saccharalis and according the in preferred embodiments the habitat is a sugar cane field, and the pest is D. saccharalis.

### Sequence Listings

The present application contains a Sequence Listing prepared in Patently included below but also submitted electronically in ST26 format which is hereby incorporated by reference in its entirety.

TABLE A

| SEQ ID NO: 1 | protein | sequence of | E11 Desaturase | from | Diatraea saccharalis |
|---|---|---|---|---|---|
| SEQ ID NO: 2 | DNA | encoding | E11 Desaturase | from | Diatraea saccharalis |
| SEQ ID NO: 3 | protein | sequence of | Desaturase | from | Dendrolimus punctatus |
| SEQ ID NO: 4 | DNA | encoding | Desaturase | from | Dendrolimus punctatus |
| SEQ ID NO: 5 | DNA | encoding | Δ9 Desaturase | from | Drosophila grimshawi |
| SEQ ID NO: 6 | DNA | encoding | Δ9 Desaturase | from | Drosophila virilis |
| SEQ ID NO: 7 | DNA | encoding | Δ9 Desaturase | from | Drosophila |
| | | | | | melanogaster |
| SEQ ID NO: 8 | DNA | encoding | Δ9 Desaturase | from | Yarrowia lipolytica |
| SEQ ID NO: 9 | DNA | encoding | Δ9 Desaturase | from | Tribolium castaneum |
| SEQ ID NO: | DNA | encoding | Δ9 Desaturase | from | Bombus lapidarius |
| 10 | | | | | |
| SEQ ID NO: | DNA | encoding | Δ9 Desaturase | from | Lobesia botrana |
| 11 | | | | | |
| SEQ ID NO: | DNA | encoding | Δ9 Desaturase | from | Saccharomyces |
| 12 | | | | | cerevisiae |
| SEQ ID NO: | DNA | encoding | Δ9 Desaturase | from | Spodoptera litura |
| 13 | | | | | |
| SEQ ID NO: | protein | sequence of | Δ9 Desaturase | from | Drosophila grimshawi |
| 14 | | | | | |
| SEQ ID NO: | protein | sequence of | Δ9 Desaturase | from | Drosophila virilis |
| 15 | | | | | |
| SEQ ID NO: | protein | sequence of | Δ9 Desaturase | from | Drosophila |
| 16 | | | | | melanogaster |
| SEQ ID NO: | protein | sequence of | Δ9 Desaturase | from | Yarrowia lipolytica |
| 17 | | | | | |
| SEQ ID NO: | protein | sequence of | Δ9 Desaturase | from | Tribolium castaneum |
| 18 | | | | | |
| SEQ ID NO: | protein | sequence of | Δ9 Desaturase | from | Bombus lapidarius |
| 19 | | | | | |
| SEQ ID NO: | protein | sequence of | Δ9 Desaturase | from | Lobesia botrana |
| 20 | | | | | |
| SEQ ID NO: | protein | sequence of | Δ9 Desaturase | from | Saccharomyces |
| 21 | | | | | cerevisiae |
| SEQ ID NO: | protein | sequence of | Δ9 Desaturase | from | Spodoptera litura |
| 22 | | | | | |
| SEQ ID NO: | DNA/RNA | sequence of | Primer | from | Artificial |
| 23 | | | | | |
| SEQ ID NO: | DNA/RNA | sequence of | Primer | from | Artificial |
| 24 | | | | | |
| SEQ ID NO: | DNA | encoding | FAR | from | Helicoverpa armigera |
| 25 | | | | | |
| SEQ ID NO: | DNA | encoding | FAR | from | Heliothis subflexa |
| 26 | | | | | |
| SEQ ID NO: | DNA | encoding | FAR | from | Heliothis virescens |
| 27 | | | | | |
| SEQ ID NO: | DNA | encoding | FAR | from | Helicoverpa assulta |
| 28 | | | | | |
| SEQ ID NO: | DNA | encoding | FAR | from | Yponomeuta rorellus |
| 29 | | | | | |
| SEQ ID NO: | DNA | encoding | FAR | from | Ostrinia nubilalis |
| 30 | | | | | |
| SEQ ID NO: | DNA | encoding | FAR | from | Agrotis segetum |
| 31 | | | | | |
| SEQ ID NO: | DNA | encoding | FAR | from | Marinobacter algicola |
| 32 | | | | | |
| SEQ ID NO: | DNA | encoding | FAR | from | Chilo suppressalis |
| 33 | | | | | |
| SEQ ID NO: | DNA | encoding | FAR | from | Spodoptera littoralis |
| 34 | | | | | |
| SEQ ID NO: | DNA | encoding | FAR | from | Spodoptera exigua |
| 35 | | | | | |
| SEQ ID NO: | DNA | encoding | FAR | from | Spodoptera exigua |
| 36 | | | | | |
| SEQ ID NO: | DNA | encoding | FAR | from | Spodoptera frugiperda |
| 37 | | | | | |
| SEQ ID NO: | DNA | encoding | FAR | from | Plutella xylostella |
| 38 | | | | | |
| SEQ ID NO: | DNA | encoding | FAR | from | Plodia interpunctella |
| 39 | | | | | |
| SEQ ID NO: | DNA | encoding | FAR | from | Amyelois transitella |
| 40 | | | | | |
| SEQ ID NO: | DNA | encoding | FAR | from | Ostrinia zea |
| 41 | | | | | |
| SEQ ID NO: | DNA | encoding | FAR | from | Trichoplusia ni |
| 42 | | | | | |
| SEQ ID NO: | DNA | encoding | FAR | from | Trichoplusia ni |
| 43 | | | | | |
| SEQ ID NO: | DNA | encoding | FAR | from | Chrysodeixis includens |
| 44 | | | | | |
| SEQ ID NO: | DNA | encoding | FAR | from | Chrysodeixis includens |
| 45 | | | | | |
| SEQ ID NO: | protein | sequence of | FAR | from | Helicoverpa armigera |
| 46 | | | | | |
| SEQ ID NO: | protein | sequence of | FAR | from | Heliothis subflexa |
| 47 | | | | | |
| SEQ ID NO: | protein | sequence of | FAR | from | Heliothis virescens |
| 48 | | | | | |
| SEQ ID NO: | protein | sequence of | FAR | from | Helicoverpa assulta |
| 49 | | | | | |
| SEQ ID NO: | protein | sequence of | FAR | from | Yponomeuta rorellus |
| 50 | | | | | |
| SEQ ID NO: | protein | sequence of | FAR | from | Ostrinia nubilalis |
| 51 | | | | | |
| SEQ ID NO: | protein | sequence of | FAR | from | Agrotis segetum |
| 52 | | | | | |
| SEQ ID NO: | protein | sequence of | FAR | from | Marinobacter algicola |
| 53 | | | | | |
| SEQ ID NO: | protein | sequence of | FAR | from | Chilo suppressalis |
| 54 | | | | | |
| SEQ ID NO: | protein | sequence of | FAR | from | Spodoptera littoralis |
| 55 | | | | | |
| SEQ ID NO: | protein | sequence of | FAR | from | Spodoptera exigua |
| 56 | | | | | |
| SEQ ID NO: | protein | sequence of | FAR | from | Spodoptera exigua |
| 57 | | | | | |
| SEQ ID NO: | protein | sequence of | FAR | from | Spodoptera frugiperda |
| 58 | | | | | |
| SEQ ID NO: | protein | sequence of | FAR | from | Plutella xylostella |
| 59 | | | | | |
| SEQ ID NO: | protein | sequence of | FAR | from | Plodia interpunctella |
| 60 | | | | | |
| SEQ ID NO: | protein | sequence of | FAR | from | Amyelois transitella |
| 61 | | | | | |
| SEQ ID NO: | protein | sequence of | FAR | from | Ostrinia zea |
| 62 | | | | | |
| SEQ ID NO: | protein | sequence of | FAR | from | Trichoplusia ni |
| 63 | | | | | |
| SEQ ID NO: | protein | sequence of | FAR | from | Trichoplusia ni |
| 64 | | | | | |
| SEQ ID NO: | protein | sequence of | FAR | from | Chrysodeixis includens |
| 65 | | | | | |
| SEQ ID NO: | protein | sequence of | FAR | from | Chrysodeixis includens |
| 66 | | | | | |
| SEQ ID NO: | protein | sequence of | Primer | from | Artificial |
| 67 | | | | | |
| SEQ ID NO: | protein | sequence of | Fatty aldehyde | from | Artificial |
| 68 | | | dehydrogenase | | |
| SEQ ID NO: | protein | sequence of | Fatty alcohol oxidase | from | Artificial |
| 69 | | | | | |
| SEQ ID NO: | protein | sequence of | Fatty alcohol oxidase | from | Artificial |
| 70 | | | | | |
| SEQ ID NO: | protein | sequence of | Glycerol-3-phosphate | from | Artificial |
| 71 | | | O-acyltransferase | | |
| SEQ ID NO: | protein | sequence of | Z11 desaturase | from | Amyelois transitella |
| 72 | | | | | |
| SEQ ID NO: | DNA | encoding | Z11 desaturase | from | Amyelois transitella |
| 73 | | | | | |
| SEQ ID NO: | protein | sequence of | Z11 desaturase | from | Spodoptera exigua |
| 74 | | | | | |
| SEQ ID NO: | DNA | encoding | Z11 desaturase | from | Spodoptera exigua |
| 75 | | | | | |
| SEQ ID NO: | protein | sequence of | Z11 desaturase | from | Spodoptera litura |
| 76 | | | | | |
| SEQ ID NO: | DNA | encoding | Z11 desaturase | from | Spodoptera litura |
| 77 | | | | | |
| SEQ ID NO: | protein | sequence of | Z11 desaturase | from | Helicoverpa zea |
| 78 | | | | | |
| SEQ ID NO: | DNA | encoding | Z11 desaturase | from | Helicoverpa zea |
| 79 | | | | | |
| SEQ ID NO: | protein | sequence of | E11 Desaturase | from | Diatraea saccharalis |
| 80 | | | | | |
| SEQ ID NO: | DNA | encoding | E11 Desaturase | from | Diatraea saccharalis |
| 81 | | | | | |
| SEQ ID NO: | protein | sequence of | Δ9 Desaturase | from | Diatraea saccharalis |
| 82 | | | | | |
| SEQ ID NO: | DNA | encoding | Δ9 Desaturase | from | Diatraea saccharalis |
| 83 | | | | | |
| SEQ ID NO: | protein | sequence of | NAD(P)H cytochrome b5 | from | Cydia pomonella |
| 84 | | | oxidoreductase | | |
| SEQ ID NO: | DNA | encoding | NAD(P)H cytochrome b5 | from | Cydia pomonella |
| 85 | | | oxidoreductase | | |
| SEQ ID NO: | DNA | | terminator | | Synthetic minimal |
| 86 | | | | | terminator scaffold |
| SEQ ID NO: | DNA | encoding | FAR | from | Diatraea saccharalis |
| 87 | | | | | |
| SEQ ID NO: | protein | sequence of | FAR | from | Diatraea saccharalis |
| 88 | | | | | |
| SEQ ID NO: | DNA | encoding | E11 Desaturase | from | Diatraea saccharalis |
| 89 | | | | | |
| SEQ ID NO: | protein | sequence of | E11 Desaturase | from | Diatraea saccharalis |
| 90 | | | | | |
| SEQ ID NO: | DNA | encoding | E11 Desaturase | from | Diatraea saccharalis |
| 91 | | | | | |
| SEQ ID NO: | protein | sequence of | E11 Desaturase | from | Diatraea saccharalis |
| 92 | | | | | |
| SEQ ID NO: | DNA | encoding | E11 Desaturase | from | Diatraea saccharalis |
| 93 | | | | | |
| SEQ ID NO: | DNA | encoding | FAR | from | Tyta alba |
| 94 | | | | | |
| SEQ ID NO: | protein | sequence of | FAR | from | Tyta alba |
| 95 | | | | | |
| SEQ ID NO: | protein | sequence of | E11 Desaturase | from | Synthetic protein |
| 96 | | | | | scaffold |
| SEQ ID NO: | DNA | encoding | E11 Desaturase | from | Synthetic protein |
| 97 | | | | | scaffold |
| SEQ ID NO: | protein | sequence of | E11 Desaturase | from | Synthetic protein |
| 98 | | | | | scaffold |
| SEQ ID NO: | DNA | encoding | E11 Desaturase | from | Synthetic protein |
| 99 | | | | | scaffold |
| SEQ ID NO: | protein | sequence of | E11 Desaturase | from | Synthetic protein |
| 100 | | | | | scaffold |
| SEQ ID NO: | DNA | encoding | E11 Desaturase | from | Synthetic protein |
| 101 | | | | | scaffold |
| SEQ ID NO: | protein | sequence of | E11 Desaturase | from | Synthetic protein |
| 102 | | | | | scaffold |
| SEQ ID NO: | DNA | encoding | E11 Desaturase | from | Synthetic protein |
| 103 | | | | | scaffold |
| SEQ ID NO: | protein | sequence of | E11 Desaturase | from | Synthetic protein |
| 104 | | | | | scaffold |
| SEQ ID NO: | DNA | encoding | E11 Desaturase | from | Synthetic protein |
| 105 | | | | | scaffold |
| SEQ ID NO: | protein | sequence of | acetyl transferase | from | Saccharomyces |
| 106 | | | | | cerevisiae |

### EXAMPLES

### Example 1—Construction of BioBricks and Plasmids

All heterologous genes were synthesized by GeneArt (Life Technologies) in codon-optimized versions for Y. lipolytica. Genes were either amplified by PCR using Phusion U Hot Start DNA Polymerase (ThermoFisher), or obtained by restriction digestions, to obtain the fragments for cloning into yeast expression vectors. The primers are listed in Table 1 and the resulting DNA fragments (BioBricks) are listed in Table 2. The PCR products or restriction digest reaction were separated on a 1%-agarose gel containing Midori Green Advance (Nippon Genetics Europe GmbH). PCR/restriction digest products of the correct size were excised from the gel and purified using the Nucleospin Gel and PCR Clean-up kit (Macherey-Nagel).

Yeast vectors with a USER cassette were linearized with FastDigest SfaAl (ThermoFisher) for 2 hours at 37° C. and then nicked with Nb.Bsml (New England Biolabs) for 1 hour at 65° C. The resulting vectors containing sticky ends were separated by gel electrophoresis, excised from the gel, and gel-purified using the Nucleospin Gel and PCR Clean-up kit (Macherey-Nagel). The DNA fragments were cloned into vectors by USER-cloning as described in (Holkenbrink, et al., 2018) (Jensen, et al., 2014). The USER reaction was transformed into chemically competent Escherichia coli DHα cells and the cells were plated on Lysogeny Broth (LB) agar plates with 100 mg/L ampicillin. The plates were incubated overnight at 37° C. and the resulting colonies were screened by colony PCR. The plasmids were purified from overnight E. coli liquid cultures and the correct cloning was confirmed by sequencing. The constructed vectors are listed in Table 3.

TABLE 1
Primers

| | | NCBI accession | |
|---|---|---|---|
| Primer ID | Template | number | Hybridises in positions |
| PR10852 | Yali0E | NC_006071 | 784691 . . . 784713 |
| PR11110 | pCfB6681* | | 2306 . . . 2336 |
| PR11111 | pCfB6681* | | 5129 . . . 5152 |
| PR14148 | Yali0E | NC_006071 | 784691 . . . 784708 |
| PR15781 | Yali0A | NC_006067 | 2188556 . . . 2188574 |
| PR14620 | Yali0A | NC_006067 | 2188565 . . . 2188546 |
| PR18214 | Yali0C | NC_006069 | 1243743 . . . 1243761 |
| PR23166 | Yali0E | NC_006071 | 2086416 . . . 2086402 |
| PR23167 | Yali0E | NC_006071 | 2085907 . . . 2085919 |
| PR23168 | Yali0E | NC_006071 | 2086480 . . . 2086497 |
| PR23169 | Yali0E | NC_006071 | 2087001 . . . 2086986 |
| PR25094 | | SEQ ID NO: 67 | |
| PR25309 | Yali0C | NC_006069 | 1244237 . . . 1244223 |
| PR25379 | SEQ ID NO: 4 | | 4 . . . 21 |
| PR25380 | SEQ ID NO: 4 | | 945 . . . 963 |
| PR25381 | | SEQ ID NO: 24 | |
| PR26831 | SEQ ID NO: 2 | | 4 . . . 21 |
| PR26832 | SEQ ID NO: 2 | | 1015 . . . 1035 |
| PR27323 | Yali0C | NC_006069 | 1244237 . . . 1244223 |
| PR27324 | Yali0C | NC_006069 | 1243743 . . . 1243761 |

*(Holkenbrink, et al., 2018)

TABLE 2
DNA fragments (BioBricks) obtained by PCR and restriction
digestion using the indicated template and primers

| | | | Forward | Reverse |
|---|---|---|---|---|
| BioBrick ID | Description | Template | primer | primer |
| BB1135 | Vector backbone | pCfB6681* | PR11110 | PR11111 |
| BB1631 | Regions downstream of Y. | pCfB4586* | PR14148 | PR15781 |
| | lipolytica pex20 and lip2 | | | |
| BB8679 | Genomic region upstream of | ST4840** | PR23167 | PR23166 |
| | integration site | | | |
| BB8680 | Genomic region downstream of | ST4840** | PR23168 | PR23169 |
| | integration site | | | |
| BB8955 | Regions downstream of Y. | pBP8662 | PR10852 | PR14620 |
| | lipolytica pex20 and lip2 | | | |
| BB9454 | Region upstream of Y. lipolytica | ST4840** | PR25309 | PR18214 |
| | tef1 | | | |
| BB9506 | Regions downstream of Y. | pBP9002 | PR25381 | PR25094 |
| | lipolytica pex20 and lip2 | | | |
| BB9507 | D. punctatus desaturase | SEQ ID NO: 4 | PR25379 | PR25380 |
| BB10122 | D. saccharalis desaturase | SEQ ID NO: 2 | PR26831 | PR26832 |
| BB10398 | region upstream of Y. lipolytica | ST4840** | PR27323 | PR27324 |
| | tef1 | | | |
| BB10439 | D. saccharalis desaturase | SEQ ID NO: 81 | | |
| BB10594 | D. saccharalis desaturase | SEQ ID NO: 83 | | |

*(Holkenbrink, et al., 2018)
**(Holkenbrink, et al., 2020)

TABLE 3
Vectors

| Plasmid | | Parent | |
|---|---|---|---|
| ID | Resistance | plasmid | BioBricks |
| pBP8662 | | | BB1135, BB8679, BB8680, |
| | | | BB1631 |
| pBP9002 | Nourseothricin | pCfB3405* | BB8955 |
| pBP9698 | Nourseothricin | pCfB3405* | BB9506 |
| pBP9700 | Nourseothricin | pBP9002 | BB9454, BB9507 |
| pBP10539 | Nourseothricin | pBP9698 | BB9454, BB10122 |
| pBP11063 | Nourseothricin | pBP10995 | BB10398, BB10439 |

*(Holkenbrink, et al., 2018)

### Example 2—Construction of Yeast Strains

Yeast strains were constructed by transformation of DNA vectors as described in (Holkenbrink, et al., 2018) (Jensen, et al., 2014). Strains were selected on yeast peptone dextrose (YPD) agar with appropriate antibiotics selection or on synthetic drop-out medium (Sigma-Adrich) lacking a specific amino acid. Correct genotype was confirmed by colony PCR and when needed by sequencing. The resulting strains are listed in Table 4.

Strains marked with “***” were constructed as follows. The indicated genes were amplified with gene-specific primers containing a 5′-overhang of: “ACTTTTTGCAGTACUAACCGCAG” in the forward primer and a 3′-overhang of “CACGCGAU” in the reverse primer. The first “ATG” of the target gene sequence was omitted. These PCR products were cloned together with BB9454 either into integrative vectors or episomal vectors as described in (Holkenbrink, et al., 2018)

Strains marked with “****” were constructed as follows. The indicated genes were cloned together with BB10398 and a synthetic minimal terminator (SEQ ID NO: 86) (ST13043) or Y. lipolytica native LIP2 terminator (ST13149, ST13247) either into integrative vectors or episomal vectors by “Golden-Gate Assembly” as described in (Pryor, J. M., et al., 2020) or by following the User's Manual of NEBridge Golden Gate Assembly Kit (New England BioLabs, Inc.). The first “ATG” of the target gene sequence was omitted. The episomal vector pBP10995 is derived from pCfB3405 (Holkenbrink et al. 2018), adopted for Golden-Gate assembly.

TABLE 4
Yeast strains

| | Overexpressed | | | Parent | |
|---|---|---|---|---|---|
| Name | gene | ID (DNA) | ID (Protein) | Strain | DNA |
| ST6629** | — | | | | |
| ST10444 | | | | ST6629 | pBP9002 |
| ST10746 | Dpu_APSQ | SEQ ID NO: 4 | SEQ ID NO: 3 | ST6629 | pBP9700 |
| ST12028 | Ds12389 | SEQ ID NO: 2 | SEQ ID NO: 1 | ST6629 | pBP10539 |
| ST12118*** | FAR1 | SEQ ID NO: 25 | SEQ ID NO: 46 | ST6629 | |
| ST12119*** | FAR4 | SEQ ID NO: 26 | SEQ ID NO: 47 | ST6629 | |
| ST12120*** | FAR5 | SEQ ID NO: 27 | SEQ ID NO: 48 | ST6629 | |
| ST12121*** | FAR6 | SEQ ID NO: 28 | SEQ ID NO: 49 | ST6629 | |
| ST12122*** | FAR8 | SEQ ID NO: 29 | SEQ ID NO: 50 | ST6629 | |
| ST12123*** | FAR9 | SEQ ID NO: 30 | SEQ ID NO: 51 | ST6629 | |
| ST12125*** | FAR12 | SEQ ID NO: 31 | SEQ ID NO: 52 | ST6629 | |
| ST12126*** | FAR42 | SEQ ID NO: 32 | SEQ ID NO: 53 | ST6629 | |
| ST12129*** | FAR13 | SEQ ID NO: 33 | SEQ ID NO: 54 | ST6629 | |
| ST12131*** | FAR15 | SEQ ID NO: 34 | SEQ ID NO: 55 | ST6629 | |
| ST12132*** | FAR16 | SEQ ID NO: 35 | SEQ ID NO: 56 | ST6629 | |
| ST12133*** | FAR17 | SEQ ID NO: 36 | SEQ ID NO: 57 | ST6629 | |
| ST12138*** | FAR22 | SEQ ID NO: 37 | SEQ ID NO: 58 | ST6629 | |
| ST12140*** | FAR27 | SEQ ID NO: 38 | SEQ ID NO: 59 | ST6629 | |
| ST12141*** | FAR28 | SEQ ID NO: 39 | SEQ ID NO: 60 | ST6629 | |
| ST12146*** | FAR33 | SEQ ID NO: 40 | SEQ ID NO: 61 | ST6629 | |
| ST12151*** | FAR49 | SEQ ID NO: 41 | SEQ ID NO: 62 | ST6629 | |
| ST12154*** | FAR38 | SEQ ID NO: 42 | SEQ ID NO: 63 | ST6629 | |
| ST12156*** | FAR40 | SEQ ID NO: 43 | SEQ ID NO: 64 | ST6629 | |
| ST12159*** | FAR52 | SEQ ID NO: 44 | SEQ ID NO: 65 | ST6629 | |
| ST12160*** | FAR47 | SEQ ID NO: 45 | SEQ ID NO: 66 | ST6629 | |
| ST12834*** | Ncb5or | SEQ ID NO: 85 | SEQ ID NO: 84 | ST6629 | |
| ST13043**** | DsaDes1 | SEQ ID NO: 81 | SEQ ID NO: 80 | ST12834 | pBP11063 |
| ST13042 | Ds12389 | SEQ ID NO: 2 | SEQ ID NO: 1 | ST12834 | pBP10539 |
| ST13046 | — | | | ST12834 | pBP9002 |
| ST13146*** | DsaDes1 | SEQ ID NO: 81 | SEQ ID NO: 80 | ST6629 | |
| ST13147*** | DsaFAR1 | SEQ ID NO: 87 | SEQ ID NO: 88 | ST13146 | |
| ST13149*** | DsaDes7 | SEQ ID NO: 83 | SEQ ID NO: 82 | ST13146 | |
| ST13151 | | | | ST13146 | pBP9002 |
| ST13152*** | FAR1 | SEQ ID NO: 25 | SEQ ID NO: 46 | ST13146 | |
| ST13162*** | FAR16 | SEQ ID NO: 35 | SEQ ID NO: 56 | ST13146 | |
| ST13247**** | DsaDes7 | SEQ ID NO: 83 | SEQ ID NO: 82 | ST6629 | |
| ST13251*** | FAR25 | SEQ ID NO: 94 | SEQ ID NO: 95 | ST13146 | |
| ST13284*** | Desat87 | SEQ ID NO: 97 | SEQ ID NO: 96 | ST6629 | |
| ST13285*** | Desat88 | SEQ ID NO: 99 | SEQ ID NO: 98 | ST6629 | |
| ST13286*** | Desat89 | SEQ ID NO: 101 | SEQ ID NO: 100 | ST6629 | |
| ST13287*** | Desat90 | SEQ ID NO: 103 | SEQ ID NO: 102 | ST6629 | |
| ST13288*** | Desat91 | SEQ ID NO: 105 | SEQ ID NO: 104 | ST6629 | |

**(Holkenbrink, et al., 2020)

### Example 3—Cultivation of Strains and Analysis of Fatty Alcohols and Fatty Acid Methyl Esters (FAME)

Y. lipolytica strains were inoculated from a YPD agar plate (10 g/L yeast extract, 10 g/L peptone, 20 g/L glucose, 15 g/L agar agar) to an initial OD600 of 0.2 into 2.5 mL YPG medium (10 g/L yeast extract, 10 g/L peptone, 40 g/L glycerol) in 24 well-plates (EnzyScreen). The plates were incubated at 28° C., shaken at 300 rpm. After 24 h, the plates were centrifuged for 5 min at 4° C. and 3,000×g. The supernatant was discarded and the cells were resuspended in 1.25 mL production medium per well (50 g/L glycerol, 5 g/L yeast extract, 4 g/L KH₂PO₄, 1.5 g/L MgSO₄, 0.2 g/L NaCl, 0.265 g/L CaCl₂·2H₂O, 2 mL/L trace elements solution: 4.5 g/L CaCl₂·2H₂O, 4.5 g/L ZnSO₄·7H2O, 3 g/L FeSO₄·7H₂O, 1 g/L H₃BO₃, 1 g/L MnCl₂·4H₂O, 0.4 g/L N Na₂MoO₄·2H₂O, 0.3 g/L CoCl₂.6H₂O, 0.1 g/L CuSO₄·5H₂O, 0.1 g/L KI, 15 g/L EDTA). The media was supplemented with antibiotics if necessary. The plate was incubated for 26 hours at 28° C., shaken at 300 rpm.

For analysis of the fatty acids, 1 mL of each vial was harvested by centrifugation for 5 min at 4° C. and 3,000×g. Each pellet was extracted with 1000 μL 1M HCl in Methanol (anhydrous). The samples were vortexed for 20 sec and placed in the 70° C. water bath for 2 h. The samples were vortexed every 30 min for 10 sec. After cooling down of the samples to room temperature, 1000 μL of 1M NaOH in Methanol (anhydrous), 500 μL of NaCl saturated H2O, 990 μL of hexane and 10 μL of 19:Me (10 mg/mL) as internal standard were added. The samples were vortexed and centrifuged for 5 min at 21° C. and 3,000×g. The upper organic phase was analyzed via gas chromatography-mass spectrometry (GC-MS). For analysis of fatty alcohols, 1 mL of each vial was harvested by centrifugation for 5 min at 4° C. and 3,000×g. Each cell pellet was extracted with 1 ml of ethyl acetate:ethanol (84:15) and 10 μL of 19:Me (10 mg/mL) was added as internal standard. The samples were vortexed for 20 sec and incubated for 1 hour at room temperature, followed by 5 min of vortexing. 300 μL of H2O was added to each sample. The samples were vortexed and centrifuged for 5 min at 21° C. and 3,000×g. The upper organic phase was analyzed via gas chromatography-mass spectrometry (GC-MS). GC-MS analyses were performed on an Agilent 7820A GC coupled to a mass selective detector Agilent 5977B. The GC was equipped with an DB Fatwax column (30 m×0.25 mm×0.25 μm), and helium was used as carrier gas. The MS was operated in electron impact mode (70 eV), scanning between m/z 30 and 400, and the injector was configured in split mode 20:1 at 220° C. Oven temperature was set to 80° C. for 1 min, then increased at a rate of 20° C./min to 210° C., followed by a hold at 210° C. for 7 min, and then increased at a rate of 20 C/min to 230° C. Compounds were identified by comparison of retention times and mass spectra of the reference compounds. Data were analyzed by the Agilent Masshunter software.

### Example 4—Production of Z9, E11-16:CoA in the Yeast Y. lipolytica

The newly identified desaturase Ds12389 from D. saccharalis was expressed in Y. lipolytica strain ST6629 (Holkenbrink et al., 2020) to generate strain ST12028. The empty expression vector (pBP9002) was transformed into the same parent strain to give ST10444 as a control strain. Cultivation of both strains and extraction of fatty acids were performed as described in Example 3.

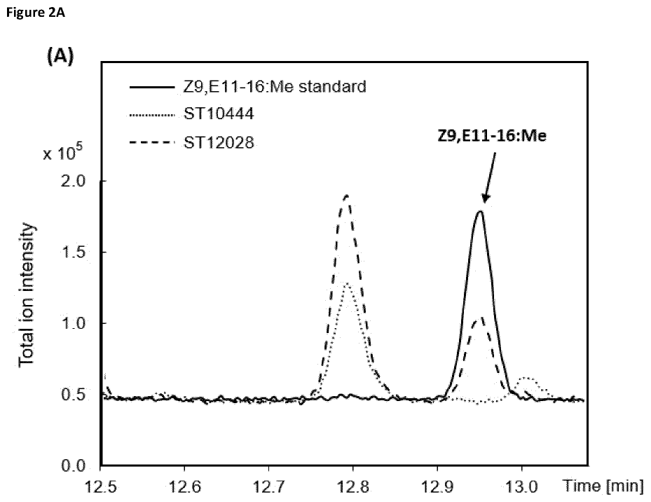

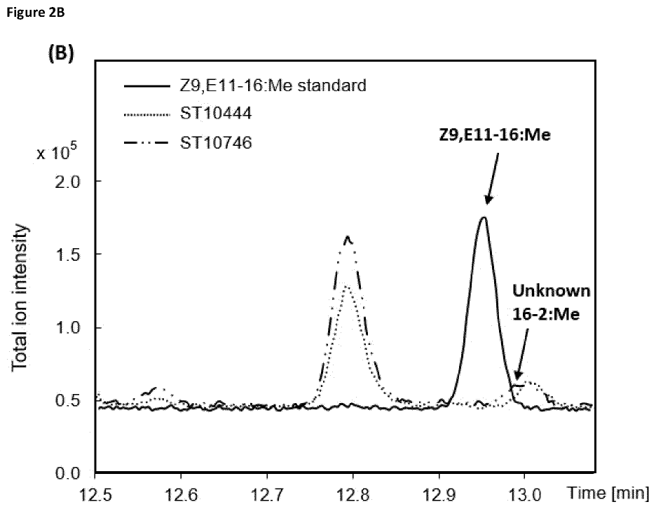

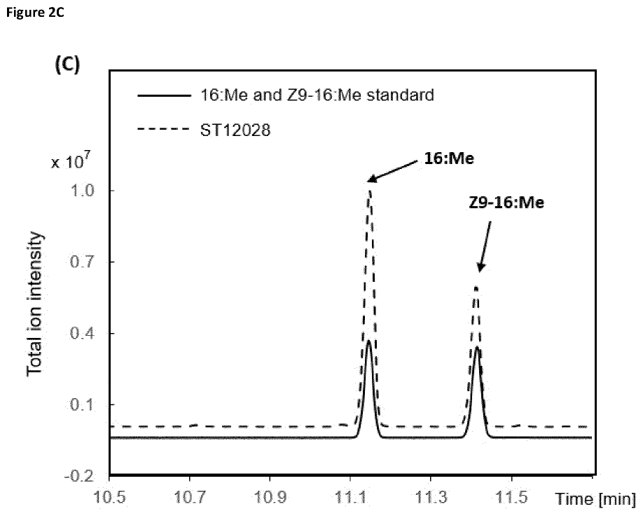

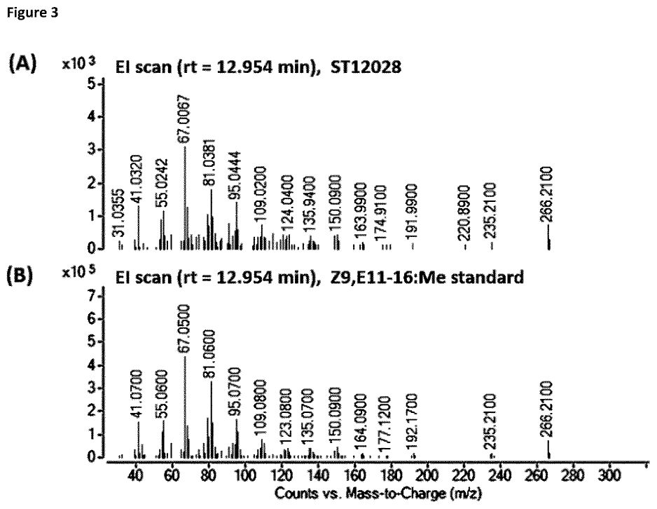

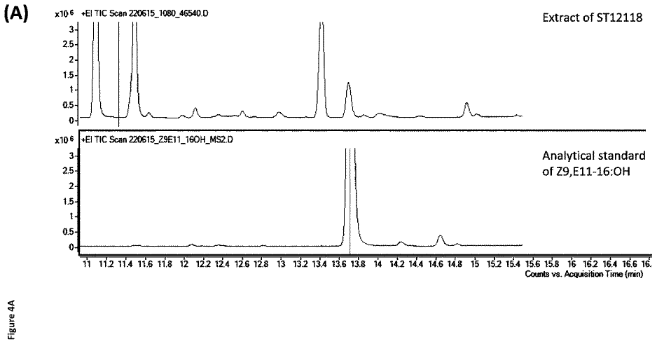

FAME extracts of strain ST12028, expressing Ds12389 (FIG. 2A, the dash line), contained a double unsaturated C16-fatty acid methyl ester (16-2:Me), which was not observed in the FAME extracts of the control strain ST10444 (FIG. 2A, the dot line). This 16-2:Me eluted at the same retention time (12.954 min) as the authentic standard of Z9, E11-16:Me (FIG. 2A, the solid line) and the mass spectrum matches the one of the Z9, E11-16:Me standard (FIG. 3). Additionally, strain ST12028 produced 16:Me and Z9-16:Me (FIG. 2C). The titers of Z9, E11-16:Me are listed in Table 5.

TABLE 5
Production of Z9, E11-16:Me in the yeast Y. lipolytica

| Strain ID | Desaturases expressed | Z9, E11-16:Me (mg/L) |
|---|---|---|
| ST10444 | — | 0 |
| ST12028 | Ds12389 | 0.43 |

### Example 5—Expression of D. punctatus Desaturases in Y. lipolytica

Desaturase Dpu_APSQ gene from D. punctatus, was expressed in Y. lipolytica strain ST6629 (Holkenbrink et al., 2020) to generate strain ST10746. Strain ST10444, carrying the empty expression vector only, serves as a control strain.

FAME extracts of strain ST10746, expressing desaturase Dpu_APSQ, did not contain any compound eluting at 12.954 min but instead trace amounts of an unknown double unsaturated C16-fatty acyl CoA with a retention time of 12.985 min was detected (FIG. 2B).

### Example 6—Production of Z9,E11-16:OH in Y. lipolytica

Desaturase Ds12389 from D. saccharalis is co-expressed with a fatty-acyl CoA reductase in Y. lipolytica. The strain is cultivated and samples analyzed as described in Example 3 and the fatty alcohol Z9,E11-16:OH is detected.

### Example 7—Production of (Z9, E11)-hexadecadienal

A mixture of primary fatty alcohols, containing 96 wt % (Z,E)-9,11-hexadecadien-1-ol (Z9,E11-16:OH), was used as a representative sample for conversion to aldehydes. Z9,E11-16:OH (560 mg) was dissolved in 1 mL of acetonitrile in a 10 mL round bottom flask with magnetic stirrer. Then 39.0 mg of Tetrakisacetonitrile copper(I) triflate (5 mol %), 16.0 mg of 2,2′-Bipyridine (Bipy) (5 mol %), 9.0 mg of 4-Hydroxy TEMPO) (2.5 mol %), 8.5 mg of N-methyl imidazole (5 mol %) were added to the reaction mixture and stirred at 30° C. for 2 hours. The reaction mixture was extracted with 10 mL heptane and the acetonitrile layer was discarded. The heptane phase was washed with 5 ml of citric acid solution (0.15 wt % in water). Then the top heptane phase was evaporated at reduced pressure until a clear residue is formed, yielding 493 mg product containing 88.0 wt % (Z9, E11)-hexadecadienal (Z9,E11-16:Ald).

### Example 8—Production of Z9,E11-16:Acid and Z9,E11-16:OH in Saccharomyces cerevisiae

The Ds12389 desaturase (SEQ ID NO:1) from D. saccharalis is cloned alone and in combination with a fatty acyl reductase into a S. cerevisiae gene expression vectors and transformed into S. cerevisiae as described in Jensen, et al., 2014. Strain cultivation and sample extraction is performed as in Example 3. A strain expressing only Ds12389 desaturase produces Z9, E11-16:Me, while the strain additionally expressing a fatty acyl reductase gene produce Z9, E11-16:OH.

### Example 9—Conversion of Z9,E11-16:Me to Z9,E11-16:OH in Y. lipolytica by Various Fatty Acyl Reductases

Fatty acyl-CoA reductases from various organisms were expressed in Y. lipolytica. The strains were cultivated and fatty alcohol samples extracted as described in Example 3 however the cultivation in YPG medium was extended from 24 to 47 hours and that 0.2 μl of Z9,E11-16:Me was added to the production medium.

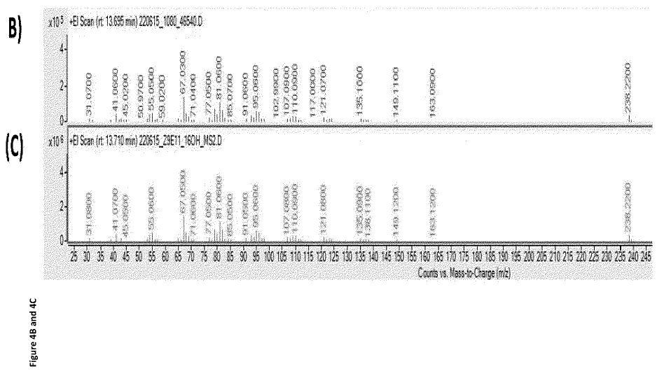

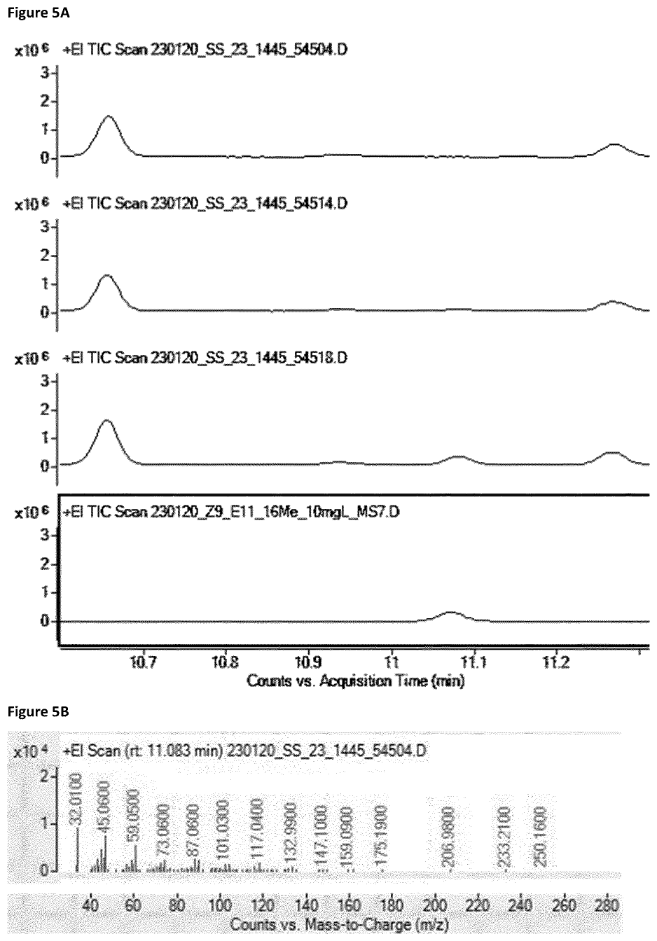

Extracts of strains ST12118-ST12123, ST12125, ST12126, ST12129, ST12131-ST12133, ST12138, ST12140, ST12141, ST12146, ST12151, ST12154, ST12156, ST12159 and ST12560 produced Z9,E11-16:OH. As an example, the GC-MS chromatogram and mass spectrum of the extract of strain ST12118 and a pure standard of Z9,E11-16:OH is shown in FIG. 4A and FIG. 4B,C, respectively. The extracts of strain ST12118 contained 5 mg/L Z9,E11-16:OH.

### Example 10—Bio-Based Pheromone Precursor Blend Production

The D. saccharalis major pheromone is (Z,E)-9,11-hexadecadienal. Minor components are (Z)-11-hexadecenal, (Z)-9-hexadecenal, and hexadecanal. The fatty alcohol precursor of both, the major and minor components, can be produced in one yeast cells. To do so, ΔE11 desaturase Ds12389 (SEQ ID NO:1) is co-expressed with any ΔZ11-16 desaturase, such as Desat16 from Amelyois transitella (SEQ ID NO: 72), Desat51 from Helicoverpa zea (SEQ ID NO:78, Desat37 from Spodoptera exigua (SEQ ID NO:74), and Desat38 from Spodoptera litura (SEQ ID NO: 76), and a suitable fatty acyl-CoA reductase known in the art for example SEQ ID NO: 46. The strains are cultivated, and fatty alcohol samples are extracted as described in Example 3.

The extract of the strain contains (Z,E)-9,11-hexadecadien-1-ol, hexadecane-1-ol, (Z)-9-hexadecen-1-ol and (Z)-11-hexadecen-1-ol.

The extract is chemically oxidized to give a composition that contains (Z,E)-9,11-hexadecadienal, hexadecanal, (Z)-9-hexadecenal and (Z)-11-hexadecenal.

The bio-based carbon content of the composition is determined by C14 radiocarbon dating.

### Example 11—Co-Expression of ΔZ9-16 Desaturases and Ds12389 (SEQ ID NO:1)

The ΔE11 desaturase Ds12389 is co-expressed with a ΔZ9-16 desaturase and optionally with a fatty acyl-CoA reductase. The strains are cultivated, and FAME and fatty alcohol samples are extracted as described in Example 3. The strain expressing Ds12389, and a ΔZ9-16 desaturase produces (Z,E)-9,11-hexadecadienoic acid methyl ester. If a fatty acyl-CoA reductase is expressed additionally (Z,E)-9,11-hexadecadien-1-ol is produced.

### Example 12—Production of Z9, E11-16:CoA in Y. lipolytica by the Expression of DsaDes1 (SEQ ID NO:80)

A newly identified desaturase DsaDes1 and Ds12389 from D. saccharalis were expressed in Y. lipolytica strain ST12834 to generate strain ST13043 and ST13042, respectively. Strain ST12834 is derived from ST6629 (Holkenbrink et al., 2020) and expressed additionally a heterologous NAD(P)H cytochrome b5 oxidoreductase Ncb5or (SEQ ID NO:84) from Cydia pomonella (WO 2022/238404 A1). The empty expression vector (pBP9002) was transformed into the same parent strain ST12834 to give ST13046 and served as a control strain. Cultivation of both strains and extraction of fatty acids were performed as described in Example 3.

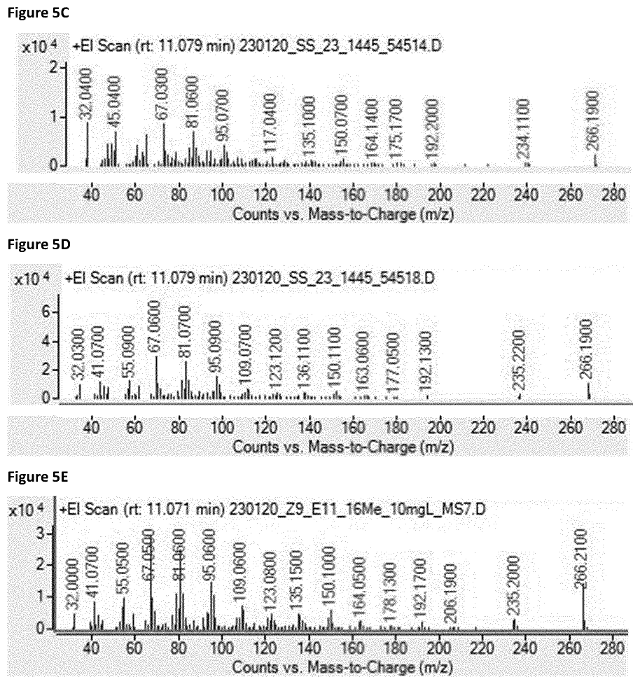

FAME extracts of strain ST13043 and ST13042, expressing DsaDes1 and Ds12389, respectively, (FIG. 5), contained a double unsaturated C16-fatty acid methyl ester (16-2:Me), which eluted at the same retention time as the authentic standard of Z9, E11-16:Me (FIG. 5). Mass spectrum of this double unsaturated 16-2:Me matches the Z9, E11-16:Me standard (FIG. 5). No Z9, E11-16:Me was detected in the FAME extracts of the control strain ST13046 (FIG. 5). The titers of Z9, E11-16:Me are listed in Table 6.

TABLE 6
Production of Z9, E11-16:Me in the yeast Y. lipolytica

| Strain | Z9, E11-16:Me (mg/L) |
|---|---|
| ST13046 | — |
| ST13043 | 20 ± 3 |
| ST13042 | 3 ± 3 |

### Example 13—Production of Z9,E11-16:OH in Y. lipolytica by the Co-Expression of DsaDes1 and Fatty Acyl-CoA Reductases

Desaturase DsaDes1 from D. saccharalis is co-expressed with a fatty-acyl CoA reductase in Y. lipolytica. The strain is cultivated and samples analyzed as described in Example 3 and the fatty alcohol Z9,E11-16:OH is detected.

### Example 14—Co-Expression of ΔZ9-16 Desaturases and DsaDes1 (SEQ ID NO:80)

The ΔE11 desaturase DsaDes1 is co-expressed with a ΔZ9-16 desaturase and optionally with a fatty acyl-CoA reductase. The strains are cultivated, and FAME and fatty alcohol samples are extracted as described in Example 3. The strain expressing DsaDes1, and a ΔZ9-16 desaturase produces (Z,E)-9,11-hexadecadienoic acid methyl ester If a fatty acyl-CoA reductase is expressed additionally (Z,E)-9,11-hexadecadien-1-ol is produced.

### Example 15—Production of Z9,E11-16:Acid and Z9,E11-16:OH in Saccharomyces cerevisiae

The desaturase Ds12389 (SEQ ID NO:1) and DsaDes1 (SEQ ID NO:80) from D. saccharalis were cloned into S. cerevisiae gene expression vectors and transformed into S. cerevisiae CEN.PK strains as described in Jensen, et al., 2014. Saccharomyces cerevisiae strains were inoculated from synthetic drop-out agar plates (lacking uracil, leucine and histidine) to an initial OD600 of 0.1-0.2 into 2.5 mL synthetic drop-out medium (lacking uracil, leucine and histidine) supplemented with 2% glucose in 24 well-plates (EnzyScreen). Sample extraction is performed as in Example 3. Derivatized FAME samples of Strain ST13093 and ST13150, expressing the desaturase Ds12389 and DsaDes1, respectively, both contained Z9, E11-16:Me (Table 7), while derivatized samples of control strain ST12515, carrying an empty expression vectors only, did not contain any Z9,E11-16:Me.

TABLE 7
Detection of Z9, E11-16:Me in derivatized FAME
samples of Saccharomyces cerevisiae strains.

| Strain ID | Gene expressed | Z9, E11-16:Me (mg/L) |
|---|---|---|
| ST12515 | — | 0 |
| ST13093 | Ds12389 | 1.6 ± 0.6 |
| ST13150 | DsaDes1 | 1.3 ± 0.6 |

### Example 16—Production of Z9,E11-16:OH in Y. lipolytica by the Co-Expression of DsaDes1 and Fatty Acyl-CoA Reductases

Desaturase DsaDes1 from D. saccharalis was co-expressed in Y. lipolytica strain ST6629 (Holkenbrink et al., 2020) in combination with fatty acyl reductases from different insect species. An empty expression vector (pBP9002) was transformed into strain ST13146, which expressing DsaDes1 only, to generate ST13151 as a control strain. Strains are cultivated and samples were analyzed as described in Example 3. The control strain ST13151 did not produce any fatty alcohols as expected. Strains ST13147, ST13152, ST13162 and ST13251, expressing the newly identified fatty acyl reductases DsaFAR1 from D. saccharalis (SEQ ID NO:88), FAR1 from Helicoverpa armigera (SEQ ID NO:46), FAR16 from Spodoptera exigua (SEQ ID NO:56) and FAR25 from Tyta alba (SEQ ID NO: 95), respectively, produced 16:OH, Z11-16:OH, Z9-16:OH and the target compound Z9,E11-16:OH (Table 8). Strain ST13147, expressing DsaFAR1, produced much lower amount of 16:OH than strains expressing FAR1 (ST13152), FAR16 (ST13162) or FAR25 (ST13251). This property is advantageous for achieving Z9,E11-16:OH production with higher purity.

TABLE 8
Production of Z9, E11-16:OH in the yeast Y. lipolytica.

| | | 16:OH | Z11-16:OH | Z9-16:OH | Z9, E11-16:OH |
|---|---|---|---|---|---|
| Strain | Gene expressed | (mg/L) | (mg/L) | (mg/L) | (mg/L) |
| ST13151 | DsaDes1 | — | — | — | — |
| ST13147 | DsaDes1, DsaFAR1 | 25.4 ± 8.2 | 1.9 ± 0.1 | 8.2 ± 1.4 | 6.8 ± 0.8 |
| ST13152 | DsaDes1, FAR1 | 199.5 ± 101.3 | 2.6 ± 0.4 | 21.5 ± 5.2 | 5.5 ± 1.8 |
| ST13162 | DsaDes1, FAR16 | 193.5 ± 38.2 | 3.0 ± 1.0 | 30.0 ± 4.9 | 8.4 ± 1.6 |
| ST13251 | DsaDes1, FAR25 | 877.0 ± 152.8 | 6.0 ± 1.7 | 6.3 ± 1.2 | 3.0 ± 0.0 |

### Example 17—Co-Expression of ΔZ9-16 Desaturases and DsaDes1

The ΔE11 desaturase DsaDes1 (SEQ ID NO:80) was co-expressed with a newly identified ΔZ9-16 desaturase DsaDes7 from Diatraea saccharalis (SEQ ID NO:82) in Y. lipolytica strain ST6629 (Holkenbrink et al., 2020) to generate ST13149. The empty expression vector (pBP9002) was transformed into strain ST13146, which expresses DsaDes1 only, to give ST13151 as a control strain. The strains were cultivated, and FAME samples were extracted as described in Example 3. When DsaDes1 was expressed alone, derivatized samples of the control strain ST13151 contained 2.0% of Z9,E11-16:Me and 10.1% of Z9-16:Me (Table 9). While derivatized FAME samples of strain ST13149, co-expressing DsaDes1 and the ΔZ9-16 desaturase DsaDes7, contained 3.3% of Z9,E11-16:Me and 15.2% of Z9-16:Me. The presence of DsaDes7 improved the purity of Z9-16:Me by 50% and Z9, E11-16:Me by 65%, respectively. If a fatty acyl-CoA reductase is expressed additionally, (Z,E)-9,11-hexadecadien-1-ol can be produced. This property is advantageous for achieving Z9,E11-16:OH production with higher purity.

TABLE 9
Effect of the ΔZ9-16 desaturase DsaDes7 on Z9-16:Me and Z9, E11-16:Me
purity in derivatized FAME samples of Yarrowia lipolytica strains.

| | Gene | Z9-16:Me | Improvement of | Z9, E11-16:Me | Improvement of |
|---|---|---|---|---|---|
| Strain | expressed | purity (%) | Z9-16:Me purity | purity (%) | Z9, E11-16:Me purity |
| ST13151 | DsaDes1 | 10.1 ± 1.2 | Reference | 2.0 ± 0.3 | Reference |
| ST13149 | DsaDes1, | 15.2 ± 2.4 | 50% | 3.3 ± 0.5 | 65% |
| | DsaDes7 | | | | |

### Example 18—Conversion of E11-16:Me to Z9,E11-16:CoA in Y. lipolytica by ΔZ9 Desaturases

The ΔZ9 desaturase DsaDes7 (SEQ ID NO:82) from D. saccharalis was expressed in Y. lipolytica strain ST6629 (Holkenbrink et al., 2020) to generate strain ST13247. The empty expression vector (pBP9002) was transformed into the same parent strain to give ST10444 as a control strain. Both strains were cultivated as described in Example 3 either with or without additional supplementation of 0.2 g/L of E11-16:Me to the cultures. After cultivation, samples were extracted and converted into fatty acid methyl ester as described in example 3. When the cultivation cultures were not supplied with E11-16:Me, derivatized FAME samples of Strain ST10444 and ST13247 did not contain any Z9,E11-16:Me. However, in derivatized samples of both strains with supplementation of E11-16:Me, Z9,E11-16:Me could be detected (Table 10). Samples of strain ST13247 had a higher purity of Z9,E11-16:Me than the control strain ST10444. These results support that both D. saccharalis ΔZ9 desaturase DsaDes7 and the native Y. lipolytica ΔZ9 desaturase OLE1 (SEQ ID NO:17) can convert E11-16:CoA into Z9,E11-16:CoA.

TABLE 10
Detection of Z9, E11-16:Me in derivatized samples of Y. lipolytica
strains with and without supplementation of E11-16:Me.

| | | E11-16:Me | Z9, | Z9, |
|---|---|---|---|---|
| | Gene | supplementation | E11-16:Me | E11-16:Me |
| Strain | expressed | to culture | (mg/L) | purity (%) |
| ST10444 | — | No | 0 | |
| ST13247 | DsaDes7 | No | 0 | |
| ST10444 | — | Yes | 2.0 ± 0.0 | 0.14 ± 0.00 |
| ST13247 | DsaDes7 | Yes | 2.5 ± 0.5 | 0.25 ± 0.06 |

### Example 19—Production of Z9,E11-16:CoA in Yarrowia lipolytica Using Alternative E11 Desaturases

Synthetic protein variants of the ΔE11 desaturase DsaDes1 were designed and expressed in Y. lipolytica strain ST6629 (Holkenbrink et al., 2020) to generate strain ST13284, ST13285, ST13286, ST13287 and ST13288. The empty expression vector (pBP9002) was transformed into the same parent strain to give ST10444 as a control strain. Cultivation of both strains and extraction of fatty acids were performed as described in Example 3. No Z9,E11-16:Me was detected from FAME extract of the control strain ST10444 as expected. FAME extracts of strains ST13284, ST13285, ST13286, ST13287 and ST13288, expressing the designed variants Desat87 (SEQ ID NO:96), Desat88 (SEQ ID NO:98), Desat89 (SEQ ID NO:100), Desat90 (SEQ ID NO:102) and Desat9l (SEQ ID NO:102), respectively, all contained the target compound Z9,E11-16:Me (Table 11). The sequence identities of synthetic protein variants can be seen in the table (Table 12). The present example demonstrates that a broad number of E11 desaturase sequence variants can be used to provide the desired E-alkene.

TABLE 11
Detection of Z9, E11-16:Me in derivatized
samples of Y. lipolytica strains.

| Strain ID | Gene expressed | Z9, E11-16:Me (mg/L) |
|---|---|---|
| ST10444 | — | 0 |
| ST13284 | Desat87 | 7.1 ± 1.7 |
| ST13285 | Desat88 | 6.2 ± 2.3 |
| ST13286 | Desat89 | 4.5 ± 1.0 |
| ST13287 | Desat90 | 8.0 ± 2.3 |
| ST13288 | Desat91 | 4.2 ± 0.4 |

TABLE 12
Sequence identity percentage between ΔE11 desaturases.

| | | 1 | 2 | 3 | 4 | 5 | 6 | 7 |
|---|---|---|---|---|---|---|---|---|
| DsaDes1 | 1 | | 97.7 | 92.2 | 86.7 | 85.3 | 84.7 | 84.3 |
| Ds12389 | 2 | 97.7 | | 90.7 | 85.0 | 84.1 | 83.2 | 82.4 |
| Desat87 | 3 | 92.2 | 90.7 | | 86.7 | 87.0 | 85.8 | 85.0 |
| Desat88 | 4 | 86.7 | 85.0 | 86.7 | | 85.8 | 84.1 | 86.4 |
| Desat89 | 5 | 85.3 | 84.1 | 87.0 | 85.8 | | 84.4 | 90.4 |
| Desat90 | 6 | 84.7 | 83.2 | 85.8 | 84.1 | 84.4 | | 84.1 |
| Desat91 | 7 | 84.3 | 82.4 | 85.0 | 86.4 | 90.4 | 84.1 | |

### Example 20—Measurement of Biobased Carbon Content

The biobased carbon content of a fatty alcohol pheromone precursor co was measured by using the analytical measurement that may be cited as “percent modern carbon (pMC)”. This is the percentage of isotope 14C measured in the sample relative to a modern reference standard (NIST 4990C). The % biobased carbon content is calculated from pMC by applying a small adjustment factor for isotope 14C in carbon dioxide in air today. Strain STSCB, engineered for production of Z9,E11-16:OH, was fermented and the product was recovered. The product sample was composed of a mix of fatty alcohols including Z9,E11-16:OH. The “percent modern carbon (pMC)” of the product sample was analyzed by the standard test method “ASTM D6866”. The percent of modern carbon was determined to be 93.80±0.33 pMC, corresponding to 94% of biobased carbon content (Table 13). This implies that the pMC of Z9,E11-16:OH contained in the product sample also was 94%.

TABLE 13
Determination of percent of modern carbon in product sample.

| Strain ID | Product sample | pMC |
|---|---|---|
| STSCB | a mix of fatty alcohols including | 93.80 ± 0.33 |
| | Z9, E11-16:OH | |

### Example 21—Production of Z9,E11-16:OH in Saccharomyces cerevisiae

The E11 desaturase DsaDes1 (SEQ ID NO:80) and the fatty acyl-CoA reductase DsaFAR1 (SEQ ID NO:88) from D. saccharalis were cloned into S. cerevisiae gene expression vectors and transformed into S. cerevisiae CEN.PK strains as described in Maury, et al., 2016 and Jensen, et al., 2014. Saccharomyces cerevisiae strains were inoculated from synthetic drop-out agar plates (lacking uracil, leucine and histidine) to an initial OD600 of 0.1-0.2 into 2.5 mL synthetic drop-out medium (lacking uracil, leucine and histidine) supplemented with 2% glucose in 24 well-plates (EnzyScreen). Sample extraction is performed as in Example 3. Strain ST13490, expressing both the DsaDes1 and DsaFAR1, produced Z9, E11-16:OH (Table 14). While the control strain ST13491, expressing DsaDes1 only, did not produce any Z9,E11-16:OH.

TABLE 14
Production of Z9, E11-16:OH in Saccharomyces cerevisiae

| | | Z9, E11-16:OH | |
|---|---|---|---|
| Strain ID | Gene expressed | (mg/L) | DNA |
| ST-13491 | DsaDes1 | 0 | pTY2-KI.URA3_TAG-PrTEF1-}DsaDes1 |
| | | | pESC-LEU-empty |
| | | | pESC-His-empty |
| ST-13490 | DsaDes1, | 2.4 ± 0.1 | pTY2-KI.URA3_TAG-PrTEF1-}DsaDes1 |
| | DsaFAR1 | | pESC-LEU-PTEF1-}DsaFAR1 |
| | | | pESC-His-PTDH3-}DsaFAR1 |

### REFERENCES

Holkenbrink, C., Dam, M. I., Kildegaard, K., Beder, J., Dom\tilde{e}nech, D. B., & Borodina, I. (2018). EasyCloneYALI: CRISPR/Cas9-Based Synthetic Toolbox for Engineering of the Yeast Yarrowia lipolytica. Biotechnol J.
Holkenbrink, C., Ding, B.-J., Wang, H.-I., Dam, M. I., Petkevicius, K., Kildegaard, K. R., . . . Borodina, I. (2020). Production of moth sex pheromones for pest control by yeast fermentation. Metab Eng., 312-321.
Jensen, N., Strucko, T., Kildegaard, K., David, F., Maury, J., Mortensen, U., . . . Borodina, I. (2014). EasyClone: method for iterative chromosomal integration of multiple genes in Saccharomyces cerevisiae. FEMS Yeast Research, 238-48.
Lienard, M., Lassance, J.-M., Wang, H.-L., Zhao, C.-H., Piskur, J., Johansson, T., & Löfstedt, C. (2010). Elucidation of the sex-pheromone biosynthesis producing 5,7-dodecadienes in Dendrolimus punctatus reveals 11- and 9-desaturases with unusual catalytic properties. Insect Biochemistry and Molecular Biology, 440-452.
Zhao, C.-H., Adolf, R., & Löfstedt, C. (2004). Sex pheromone biosynthesis in the pine caterpillar moth, Dendrolimus punctatus: pathways leading to Z5-monoene and 5,7-conjugated diene components. Insect Biochemistry and Molecular Biology, 261-271.
Da Silva, M., Cortes, A., Svensson, G., Löfstedt, C., Lima, E., & Zarbin, P. (2021). Identification of two additional behaviorally active gland constituents of female Diatraea saccharalis (Fabricius) (Lepidoptera Crambidae). Journal of the Brazilian Chemical Society.
Holkenbrink, C., Dam, M. I., Kildegaard, K., Beder, J., Dom\tilde{e}nech, D. B., & Borodina, I. (2018). EasyCloneYALI: CRISPR/Cas9-Based Synthetic Toolbox for Engineering of the Yeast Yarrowia lipolytica. Biotechnol J.
Holkenbrink, C., Ding, B.-J., Wang, H.-I., Dam, M. I., Petkevicius, K., Kildegaard, K. R., . . . Borodina, I. (2020). Production of moth sex pheromones for pest control by yeast fermentation. Metab Eng., 312-321.
Jensen, N., Strucko, T., Kildegaard, K., David, F., Maury, J., Mortensen, U., . . . Borodina, I. (2014). EasyClone: method for iterative chromosomal integration of multiple genes in Saccharomyces cerevisiae. FEMS Yeast Research, 238-48.
Kalinova, B., Kindl, J., Hovorka, O., Hoskovec, M., & Svatos, A. (2005). (11Z)-hexadec-11-enal enhances the attractiveness of Diatraea saccharalis main pheromone component in wind tunnel experiments. Journal of Applied Entomology.
Lienard, M., Lassance, J.-M., Wang, H.-L., Zhao, C.-H., Piskur, J., Johansson, T., & Löfstedt, C. (2010). Elucidation of the sex-pheromone biosynthesis producing 5,7-dodecadienes in Dendrolimus punctatus reveals 11- and 9-desaturases with unusual catalytic properties. Insect Biochemistry and Molecular Biology, 440-452.
Svatos, A., Kalinova, B., Kindl, J., Kuldova, J., Hovorka, O., Rufino Do Nascimento, R., & Oldham, N. (2001). Chemical Characterization and Synthesis of the Major Component of the Sex Pheromone of the Sugarcane Borer Diatraea saccharalis. Collect. Czech. Chem. Commun., 1682-1690.
Zhao, C.-H., Adolf, R., & Löfstedt, C. (2004). Sex pheromone biosynthesis in the pine caterpillar moth, Dendrolimus punctatus: pathways leading to Z5-monoene and 5,7-conjugated diene components. Insect Biochemistry and Molecular Biology, 261-271.
Pryor, J. M., Potapov, V., Kucera, R. B., Bilotti, K., Cantor, E. J., Lohman, G. J. S. (2020). Enabling one-pot Golden Gate assemblies of unprecedented complexity using data-optimized assembly design. PloS ONE 15(9): e0238592.

### Items of the Disclosure

The present disclosure further provides the following embodiments and items:

Item 1. An E11 fatty acyl-CoA desaturase (E11 desaturase) comprising an amino acid sequence having at least 50% identity to the of the E11 desaturase comprised in SEQ ID NO: 1 or SEQ ID NO: 80.

Item 2. The E11 desaturase of item 1 wherein the E11 desaturase in the presence of a (Z)-9-hexadecenoyl-CoA substrate introduces a double bond in a E configuration in position 11 thereby producing a (Z,E)-9,11-hexadecadienoyl-CoA.

Item 3. A polynucleotide sequence codon optimized for heterologous expression encoding the E11 desaturase of item 1 having a DNA sequence comprised in SEQ NO:2 or SEQ ID NO: 81, or a homologue thereof including variations due to the degeneracy of the genetic code.

Item 4. A polynucleotide construct comprising the polynucleotide sequence of item 3 operably linked to one or more control sequences.

Item 5. The polynucleotide construct of item 4 wherein the control sequence is heterologous to the polynucleotide.

Item 6. A vector comprising the polynucleotide construct of item 4 or 5.

Item 7. A genetically engineered microbial cell producing a (Z,E)-9,11-hexadecadienoyl-CoA said cell heterologously expressing the E11 desaturase of item 1 or 2 which in the presence of a (Z)-9-hexadecenoyl-CoA substrate introduces a double bond in a E configuration in position 11 in the (Z)-9-hexadecenoyl-CoA substrate and thereby produces the (Z,E)-9,11-hexadecadienoyl-CoA having a double bond in position 11 in E configuration.

Item 8. The cell of item 7 further comprising an operative biosynthetic pathway converting the (Z,E)-9,11-hexadecadienoyl-CoA into a target compound selected from
a) (Z,E)-9,11-hexadecadien-1-ol;
b) (Z,E)-9,11-hexadecadienal; and/or
c) (Z,E)-9,11-hexadecadienyl acetate;
   said pathway expressing one or more pathway polypeptides selected from:
a) an alcohol-forming fatty acyl-CoA reductase (FAR) converting (Z,E)-9,11-hexadecadienoyl-CoA into (Z,E)-9,11-hexadecadien-1-ol;
b) an acetyltransferase converting (Z,E)-9,11-hexadecadien-1-ol into (Z,E)-9,11-hexadecadienyl acetate;
c) an alcohol dehydrogenase converting (Z,E)-9,11-hexadecadien-1-ol into (Z,E)-9,11-hexadecadienal; and/or
d) a fatty alcohol oxidase converting (Z,E)-9,11-hexadecadien-1-ol into (Z,E)-9,11-hexadecadienal.

Item 9. The cell of item 8 wherein
a) the FAR is at least 70% identical to the FAR comprised in SEQ ID NO: 45, 46, 47, 48, 49, 50, 51, 52, 53, 54, 55, 56, 57, 58, 59, 60, 61, 62, 63, 64, or 65;
b) the acetyltransferase is at least 70% identical to the acetyltransferase comprised in SEQ ID NO: 71;
c) the alcohol dehydrogenase is at least 70% identical to the alcohol dehydrogenase comprised in SEQ ID NO: 68; and
d) the fatty alcohol oxidase is at least 70% identical to the fatty alcohol oxidase comprised in SEQ ID NO: 69 or 70.

Item 10. The cell of item 7 to 9 further comprising an operative biosynthetic pathway producing (Z)-9-hexadecenoyl-CoA substrate, said pathway expressing one or more heterologous Δ9 desaturases which in the presence of a hexadecenoyl-CoA substrate introduces a double bond in the hexadecenoyl-CoA substrate in a Z configuration in position 9.

Item 11. The cell of item 10 wherein the Δ9 desaturase is the at least 70% identical to the Δ9 desaturase comprised in SEQ ID NO: 14, 15, 16, 17, 18, 19, 20, 21, 22, or 82.

Item 12. The cell of item 7 to 11, wherein one or more native or endogenous genes are attenuated, disrupted and/or deleted.

Item 13. The cell of item 7 to 12, wherein one or more pathway genes are overexpressed.

Item 14. The cell of item 7 to 13 further genetically modified to provide an increased amount of a substrate for at least one enzyme of the (Z,E)-9,11-hexadecadienal pathway.

Item 15. The cell of item 7 to 14, further genetically modified to exhibit increased tolerance towards one or more substrates, intermediates, or product molecules from the (Z,E)-9,11-hexadecadienal pathway.

Item 16. The cell of item 7 to 15, comprising at least two copies of one or more genes in the (Z,E)-9,11-hexadecadienal pathway.

Item 17. The host cell of item 7 to 16, wherein the host cell is a fungal cell.

Item 18. The host cell of item 17, wherein the fungal cell is a yeast cell.

Item 19. The host cell of item 18, wherein the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.

Item 20. The cell of item 17 wherein the fungal cell is a filamentous fungal cell selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

Item 21. A genetically engineered yeast cell producing (Z,E)-9,11-hexadecadienoyl-CoA and (Z,E)-9,11-hexadecadien-1-ol, said cell producing hexadecanoyl-CoA and expressing
a. a Δ9 desaturase catalyzing the formation of a double bond in a Z configuration in position 9 in the hexadecanoyl-CoA thereby producing a (Z)-9-hexadecenoyl-CoA;
b. an E11 desaturase catalyzing the formation of a double bond in a E configuration in position 11 in the (Z)-9-hexadecenoyl-CoA thereby producing a (Z,E)-9,11-hexadecadienoyl-CoA; and
c. an alcohol-forming fatty acyl-CoA reductase (FAR) converting the (Z,E)-9,11-hexadecadienoyl-CoA into (Z,E)-9,11-hexadecadien-1-ol.

Item 22. The yeast cell of item 21 further expressing one or more enzymes selected from:
a. a Z11 desaturase catalyzing the formation of a double bond in a Z configuration in position 11 in the hexadecanoyl-CoA thereby producing a (Z)-11-hexadecenoyl-CoA;
b. one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol.

Item 23. The yeast cell of item 21 or 22 wherein the
a) E11 desaturase has an amino acid sequence comprised in the E11 desaturase of SEQ ID NO: 1 or 80;
b) Δ9 desaturase has an amino acid sequence comprised in the Δ9 desaturase of SEQ ID NO: 14, 15, 16, 17, 18, 19, 20, 21, 22, or 82;
c) Z11 desaturase has an amino acid sequence comprised in the Z11 desaturase of SEQ ID NO: 72, 74, 76, or 78; and
d) alcohol-forming fatty acyl-CoA reductases (FAR) has an amino acid sequence comprised in the FAR of SEQ ID NO: 45, 46, 47, 48, 49, 50, 51, 52, 53, 54, 55, 56, 57, 58, 59, 60, 61, 62, 63, 64, or 65.

Item 24. The yeast cell of item 21 or 23, wherein the yeast cell is of the species Saccharomyces cerevisiae, or Yarrowia lipolytica.

Item 25. A cell culture comprising the genetically engineered microbial cell of item 7 to 24 and a growth medium.

Item 26. A method for producing a target compound selected from:
a) (Z,E)-9,11-hexadecadienoyl-CoA
b) (Z,E)-9,11-hexadecadien-1-ol;
c) (Z,E)-9,11-hexadecadienal; and/or
d) (Z,E)-9,11-hexadecadienyl acetate;
   said method comprising culturing the cell culture of item 25 at conditions allowing the cell culture to produce the target compound, and optionally recovering and/or isolating the target compound.

Item 27. The method of item 26, comprising feeding the cell culture exogenously with one or more substrates or precursors of the target compound pathway.

Item 28. The method of item 26 to 27, wherein one or more steps of producing the target compound is performed in vitro.

Item 29. The method of item 26 to 28, wherein the in vitro performed step comprises reducing (Z,E)-9,11-hexadecadienoyl-CoA into (Z,E)-9,11-hexadecadien-1-ol using a reductase enzyme (FAR).

Item 30. The method of item 26 to 28, wherein the in vitro performed step comprises chemically or enzymatically reducing a (Z,E)-9,11-hexadecadienoic acid into (Z,E)-9,11-hexadecadien-1-ol.

Item 31. The method of item 26 to 29, wherein the in vitro performed step comprises chemical or enzymatic oxidization of (Z,E)-9,11-hexadecadien-1-ol into (Z,E)-9,11-hexadecadienal.

Item 32. The method of item 26 to 29, wherein the in vitro performed step comprises chemical or enzymatic acetylation of (Z,E)-9,11-hexadecadien-1-ol into (Z,E)-9,11-hexadecadienyl acetate.

Item 33. The method of any one of item 26 to 32, further comprising recovering the target compound and mixing it with one or more carriers, agents, additives, adjuvants and/or excipients to produce a biopesticide composition.

Item 34. The method of item 33 wherein the one or more carriers, agents, additives, adjuvants and/or excipients comprises a protective agent comprising a conjugated sulfur, which protects the target compound from being converted into an acid.

Item 35. The method of item 34 wherein the protective agent comprises a compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione.

Item 36. The method of item 34 to 35 comprising mixing in at least 10 mg of the protective agent per gram aldehyde and/or alcohol.

Item 37. The method of item 33 to 37, wherein the one or more carriers, agents, additives, adjuvants and/or excipients comprises a carrier facilitating slow release of the target compound, optionally being (i) a polymeric substrate selected from plastic, wax emulsion, oil emulsion, or microcapsules and/or (ii) zeolite.

Item 38. A method for producing a biopesticide composition comprising
(I) culturing a genetically engineered yeast cell producing hexadecanoyl-CoA and expressing
   a. a Δ9 desaturase catalyzing the formation of a double bond in a Z configuration in position 9 in the hexadecanoyl-CoA and thereby producing a (Z)-9-hexadecenoyl-CoA;
   b. an E11 desaturase catalyzing the formation of a double bond in a E configuration in position 11 in the (Z)-9-hexadecenoyl-CoA and thereby producing a (Z,E)-9,11-hexadecadienoyl-CoA;
   c. an alcohol-forming fatty acyl-CoA reductase (FAR) converting the (Z,E)-9,11-hexadecadienoyl-CoA into (Z,E)-9,11-hexadecadien-1-ol;
(II) enzymatically or chemically converting the (Z,E)-9,11-hexadecadien-1-ol to (Z,E)-9,11-hexadecadienal; and
(III) optionally recovering and/or isolating the (Z,E)-9,11-hexadecadienal and optionally one or more precursors thereof.

Item 39. The method of item 38, wherein the genetically engineered yeast cell further expresses one or more enzymes selected from:
a. a Z11 desaturase catalyzing the formation of a double bond in a Z configuration in position 11 in the hexadecanoyl-CoA and thereby producing a (Z)-11-hexadecenoyl-CoA;
b. one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol;
   and the method further comprises enzymatically or chemically converting the hexadecan-1-ol into hexadecanal, the (Z)-9-hexadecen-1-ol into (Z)-9-hexadecenal and the (Z)-11-hexadecen-1-ol and the (Z)-11-hexadecen-1-ol into (Z)-11-hexadecenal; and/or optionally recovering and/or isolating the hexadecanal, the (Z)-9-hexadecenal and the (Z)-11-hexadecenal and optionally one or more precursors thereof.

Item 40. The method of item 38 to 39, further comprising mixing the recovered (Z,E)-9,11-hexadecadienal, (Z)-9-hexadecenal, (Z)-11-hexadecenal, hexadecanal and optionally one or more precursors thereof with one or more carriers, agents, additives, adjuvants and/or excipients to produce the biopesticide composition.

Item 41. The method of item 40 wherein the carriers, agents, additives, adjuvants and/or excipients comprise one or more compounds selected from
a) a protective agent comprising a conjugated sulfur compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione, which protects the target compound from being converted into an acid; and/or
b) a carrier facilitating slow release of the (Z,E)-9,11-hexadecadienal, (Z)-9-hexadecenal, (Z)-11-hexadecenal and/or hexadecanal from the mixture, optionally being (i) a polymeric substrate selected from plastic, wax emulsion, oil emulsion, or microcapsules and/or (ii) a zeolite.

Item 42. A biopesticide composition comprising a target compound selected from (Z,E)-9,11-hexadecadienal, and optionally one or more compounds selected from (Z)-9-hexadecenal, (Z)-11-hexadecenal and/or hexadecanal, in combination with one or more carriers, agents, additives, adjuvants and/or excipients.

Item 43. The biopesticide composition of item 42, further comprising at least trace amounts of one or more compounds selected from hexadecan-1-ol, (Z)-9-hexadecen-1-ol, (Z)-11-hexadecen-1-ol, and (Z,E)-9,11-hexadecadien-1-ol, and optionally other metabolites of the cell culture.

Item 44. The biopesticide composition further comprising (Z,E)-9,11-hexadecandienyl acetate.

Item 45. The biopesticide composition of item 42 to 44 wherein one or more of the hexadecanoyl-CoA, (Z)-9-hexadecenoyl-CoA, (Z)-11-hexadecenoyl-CoA, (Z,E)-9,11-hexadecadienoyl-CoA, hexadecan-1-ol, (Z)-9-hexadecen-1-ol, (Z)-11-hexadecen-1-ol, and (Z,E)-9,11-hexadecadien-1-ol are obtained from the method of item 26 to 41 and optionally the composition comprises one or more further compounds or metabolites from the cell culture of item 25.

Item 46. The biopesticide composition of item 42 to 45 wherein the concentration of the target compound is at least 1 mg/kg composition.

Item 47. The composition of item 42 to 46 further comprising a protective agent comprising a conjugated sulfur, which protects the target compound from being converted into an acid.

Item 48. The composition of item 47 wherein the protective agent comprises a compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione.

Item 49. The composition of item 47 to 48 comprising at least 10 mg of the protective agent per gram aldehyde and/or alcohol.

Item 50. The composition of item 42 to 49, further comprising a carrier facilitating slow release of the target compound, optionally being (i) a polymeric substrate selected from plastic, wax emulsion, oil emulsion, or microcapsules and/or (ii) zeolite.

Item 51. A method of controlling or monitoring a pest comprising distributing the composition of item 42 to 50 in a habitat for the pest and allowing the target compound to control the pest.

Item 52. The method of item 51, wherein the habitat is a sugar cane field, and the pest is Diatraea saccharalis.

1. An E11 fatty acyl-CoA desaturase (E11 desaturase) comprising an amino acid sequence having at least 50% identity to the E11 desaturase comprised in SEQ ID NO: 1, SEQ ID NO: 80, SEQ ID NO: 90, SEQ ID NO: 92, SEQ ID NO: 96, SEQ ID NO: 98, SEQ ID NO: 100, SEQ ID NO: 102, or SEQ ID NO: 104.

2. The E11 desaturase of claim 1 wherein the E11 desaturase in the presence of a (Z)-9-hexadecenoyl-CoA substrate introduces a double bond in a E configuration in position 11 thereby producing a (Z,E)-9,11-hexadecadienoyl-CoA.

3. A polynucleotide sequence codon optimized for heterologous expression encoding the E11 desaturase of claim 1 having a DNA sequence comprised in SEQ NO:2, SEQ ID NO: 81, SEQ ID NO: 91, SEQ ID NO: 93, SEQ ID NO: 97, SEQ ID NO: 99, SEQ ID NO: 101, SEQ ID NO: 103, SEQ ID NO: 105, or a homologue thereof including variations due to the degeneracy of the genetic code.

4. A polynucleotide construct comprising the polynucleotide sequence of claim 3 operably linked to one or more control sequences.

5. The polynucleotide construct of claim 4 wherein the control sequence is heterologous to the polynucleotide.

6. A vector comprising the polynucleotide construct of claim 4 or 5.

7. A genetically engineered microbial cell producing a (Z,E)-9,11-hexadecadienoyl-CoA said cell heterologously expressing the E11 desaturase of claim 1 or 2 which in the presence of a (Z)-9-hexadecenoyl-CoA substrate introduces a double bond in a E configuration in position 11 in the (Z)-9-hexadecenoyl-CoA substrate and thereby produces the (Z,E)-9,11-hexadecadienoyl-CoA having a double bond in position 11 in E configuration.

8. The cell of claim 7 further comprising an operative biosynthetic pathway converting the (Z,E)-9,11-hexadecadienoyl-CoA into a target compound selected from
a) (Z,E)-9,11-hexadecadien-1-ol;
b) (Z,E)-9,11-hexadecadienal; and/or
c) (Z,E)-9,11-hexadecadienyl acetate;
said pathway expressing one or more pathway polypeptides selected from:
d) an alcohol-forming fatty acyl-CoA reductase (FAR) converting (Z,E)-9,11-hexadecadienoyl-CoA into (Z,E)-9,11-hexadecadien-1-ol;
e) an acetyltransferase converting (Z,E)-9,11-hexadecadien-1-ol into (Z,E)-9,11-hexadecadienyl acetate;
f) a fatty alcohol oxidase converting (Z,E)-9,11-hexadecadien-1-ol into (Z,E)-9,11-hexadecadienal.

9. The cell of claim 8 wherein
a) the FAR is at least 70% identical to the FAR comprised in SEQ ID NO: 46, 47, 48, 49, 50, 51, 52, 53, 54, 55, 56, 57, 58, 60, 61, 62, 63, 64, 66, 88, or 95;
b) the acetyltransferase is at least 70% identical to the acetyltransferase comprised in SEQ ID NO: 106;
c) the fatty alcohol oxidase is at least 70% identical to the fatty alcohol oxidase comprised in SEQ ID NO: 70.

10. The cell of claim 7 to 9 further comprising an operative biosynthetic pathway producing (Z)-9-hexadecenoyl-CoA substrate, said pathway expressing one or more heterologous Δ9 desaturases which in the presence of a hexadecenoyl-CoA substrate introduces a double bond in the hexadecenoyl-CoA substrate in a Z configuration in position 9.

11. The cell of claim 10 wherein the Δ9 desaturase is the at least 70% identical to the Δ9 desaturase comprised in SEQ ID NO: 14, 15, 16, 17, 18, 19, 20, 21, 22, or 82.

12. The cell of claim 7 to 11, wherein one or more native or endogenous genes are attenuated, disrupted and/or deleted.

13. The cell of claim 7 to 12, wherein one or more pathway genes are overexpressed.

14. The cell of claim 7 to 13 further genetically modified to provide an increased amount of a substrate for at least one enzyme of the (Z,E)-9,11-hexadecadienal pathway.

15. The cell of claim 7 to 14, further genetically modified to exhibit increased tolerance towards one or more substrates, intermediates, or product molecules from the (Z,E)-9,11-hexadecadienal pathway.

16. The cell of claim 7 to 15, comprising at least two copies of one or more genes in the (Z,E)-9,11-hexadecadienal pathway.

17. The cell of claim 7 to 15, comprising at least two copies of one or more genes in the (Z,E)-9,11-hexadecadien-1-ol pathway.

18. The host cell of claim 7 to 17, wherein the host cell is a fungal cell.

19. The host cell of claim 18, wherein the fungal cell is a yeast cell.

20. The host cell of claim 19, wherein the yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Pichia pastoris, Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.

21. The cell of claim 18 wherein the fungal cell is a filamentous fungal cell selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

22. A genetically engineered yeast cell producing (Z,E)-9,11-hexadecadienoyl-CoA and (Z,E)-9,11-hexadecadien-1-ol, said cell producing hexadecanoyl-CoA and expressing
a. a Δ9 desaturase catalyzing the formation of a double bond in a Z configuration in position 9 in the hexadecanoyl-CoA thereby producing a (Z)-9-hexadecenoyl-CoA;
b. an E11 desaturase catalyzing the formation of a double bond in a E configuration in position 11 in the (Z)-9-hexadecenoyl-CoA thereby producing a (Z,E)-9,11-hexadecadienoyl-CoA; and
c. an alcohol-forming fatty acyl-CoA reductase (FAR) converting the (Z,E)-9,11-hexadecadienoyl-CoA into (Z,E)-9,11-hexadecadien-1-ol.

23. The yeast cell of claim 22 further expressing one or more enzymes selected from:
a. a Z11 desaturase catalyzing the formation of a double bond in a Z configuration in position 11 in the hexadecanoyl-CoA thereby producing a (Z)-11-hexadecenoyl-CoA;
b. one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol.

24. The yeast cell of claim 22 or 23 wherein the
a) E11 desaturase has at least 70% sequence identity to an amino acid sequence comprised in the E11 desaturase of SEQ ID NO: 1, 80, 90, 92, 96, 98, 100, 102, or 104;
b) Δ9 desaturase has at least 70% sequence identity to an amino acid sequence comprised in the Δ9 desaturase of SEQ ID NO: 14, 15, 16, 17, 18, 19, 20, 21, 22, or 82;
c) Z11 desaturase has at least 70% sequence identity to an amino acid sequence comprised in the Z11 desaturase of SEQ ID NO: 72, 74, 76, or 78; and/or
d) alcohol-forming fatty acyl-CoA reductases (FAR) has at least 70% sequence identity to an amino acid sequence comprised in the FAR of SEQ ID NO: 46, 47, 48, 49, 50, 51, 52, 53, 54, 55, 56, 57, 58, 60, 61, 62, 63, 64, 66, 88, or 95.

25. The yeast cell of claim 22 to 24 wherein the
a) E11 desaturase has an amino acid sequence comprised in the E11 desaturase of SEQ ID NO: 1, 80, 90, 92, 96, 98, 100, 102, or 104;
b) Δ9 desaturase has an amino acid sequence comprised in the Δ9 desaturase of SEQ ID NO: 14, 15, 16, 17, 18, 19, 20, 21, 22, or 82;
c) Z11 desaturase has an amino acid sequence comprised in the Z11 desaturase of SEQ ID NO: 72, 74, 76, or 78; and/or
d) alcohol-forming fatty acyl-CoA reductases (FAR) has an amino acid sequence comprised in the FAR of SEQ ID NO: 46, 47, 48, 49, 50, 51, 52, 53, 54, 55, 56, 57, 58, 60, 61, 62, 63, 64, 66, 88, or 95.

26. The yeast cell of claim 22 to 25, wherein the yeast cell is of the species Saccharomyces cerevisiae, or Yarrowia lipolytica.

27. A cell culture comprising the genetically engineered microbial cell of claim 7 to 26 and a growth medium.

28. A method for producing a target compound selected from:
a) (Z,E)-9,11-hexadecadienoyl-CoA
b) (Z,E)-9,11-hexadecadien-1-ol;
c) (Z,E)-9,11-hexadecadienal; and/or
d) (Z,E)-9,11-hexadecadienyl acetate;
said method comprising culturing the cell culture of claim 27 at conditions allowing the cell culture to produce the target compound, and optionally recovering and/or isolating the target compound.

29. The method of claim 28, comprising feeding the cell culture exogenously with one or more substrates or precursors of the target compound pathway.

30. The method of claim 28 to 29, wherein one or more steps of producing the target compound is performed in vitro.

31. The method of claim 28 to 30, wherein the in vitro performed step comprises reducing (Z,E)-9,11-hexadecadienoyl-CoA into (Z,E)-9,11-hexadecadien-1-ol using a reductase enzyme (FAR).

32. The method of claim 28 to 30, wherein the in vitro performed step comprises chemically or enzymatically reducing a (Z,E)-9,11-hexadecadienoic acid into (Z,E)-9,11-hexadecadien-1-ol.

33. The method of claim 28 to 31, wherein the in vitro performed step comprises chemical or enzymatic oxidation of (Z,E)-9,11-hexadecadien-1-ol into (Z,E)-9,11-hexadecadienal.

34. The method of claim 28 to 31, wherein the in vitro performed step comprises chemical or enzymatic acetylation of (Z,E)-9,11-hexadecadien-1-ol into (Z,E)-9,11-hexadecadienyl acetate.

35. The method of any one of claim 28 to 34, further comprising recovering the target compound and mixing it with one or more carriers, agents, additives, adjuvants and/or excipients to produce a biopesticide composition.

36. The method of claim 35, wherein the one or more carriers, agents, additives, adjuvants and/or excipients comprises a protective agent comprising a conjugated sulfur, which protects the target compound from being converted into an acid.

37. The method of claim 36, wherein the protective agent comprises a compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione.

38. The method of claim 36 to 37 comprising mixing in at least 10 mg of the protective agent per gram aldehyde and/or alcohol.

39. The method of claim 35 to 38, wherein the one or more carriers, agents, additives, adjuvants and/or excipients comprises a carrier facilitating slow release of the target compound, optionally being (i) a polymeric substrate selected from plastic, wax emulsion, oil emulsion, or microcapsules and/or (ii) zeolite.

40. A method for producing a biopesticide composition comprising the steps:
(I) culturing a genetically engineered yeast cell producing hexadecanoyl-CoA and expressing
a. a Δ9 desaturase catalyzing the formation of a double bond in a Z configuration in position 9 in the hexadecanoyl-CoA and thereby producing a (Z)-9-hexadecenoyl-CoA;
b. an E11 desaturase catalyzing the formation of a double bond in a E configuration in position 11 in the (Z)-9-hexadecenoyl-CoA and thereby producing a (Z,E)-9,11-hexadecadienoyl-CoA;
c. an alcohol-forming fatty acyl-CoA reductase (FAR) converting the (Z,E)-9,11-hexadecadienoyl-CoA into (Z,E)-9,11-hexadecadien-1-ol;
(II) enzymatically or chemically converting the (Z,E)-9,11-hexadecadien-1-ol to (Z,E)-9,11-hexadecadienal; and
(III) optionally recovering and/or isolating the (Z,E)-9,11-hexadecadienal and optionally one or more precursors thereof.

41. The method of claim 40, wherein the genetically engineered yeast cell further expresses one or more enzymes selected from:
a. a Z11 desaturase catalyzing the formation of a double bond in a Z configuration in position 11 in the hexadecanoyl-CoA and thereby producing a (Z)-11-hexadecenoyl-CoA;
b. one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol;
and the method further comprises enzymatically or chemically converting the hexadecan-1-ol into hexadecanal, the (Z)-9-hexadecen-1-ol into (Z)-9-hexadecenal and the (Z)-11-hexadecen-1-ol and the (Z)-11-hexadecen-1-ol into (Z)-11-hexadecenal; and/or optionally recovering and/or isolating the hexadecanal, the (Z)-9-hexadecenal and the (Z)-11-hexadecenal and optionally one or more precursors thereof.

42. A method for producing a biopesticide composition comprising the steps:
(I) culturing a genetically engineered yeast cell producing hexadecanoyl-CoA and expressing
a. an E11 desaturase catalyzing the formation of a double bond in a E configuration in position 11 in the hexadecanoyl-CoA and thereby producing an (E)-11-hexadecenoyl-CoA;
b. a Δ9 desaturase catalyzing the formation of a double bond in a Z configuration in position 9 in the (E)-11-hexadecenoyl-CoA and thereby producing a (Z,E)-9,11-hexadecadienoyl-CoA;
c. an alcohol-forming fatty acyl-CoA reductase (FAR) converting the (Z,E)-9,11-hexadecadienoyl-CoA into (Z,E)-9,11-hexadecadien-1-ol;
(II) enzymatically or chemically converting the (Z,E)-9,11-hexadecadien-1-ol to (Z,E)-9,11-hexadecadienal; and
(III) optionally recovering and/or isolating the (Z,E)-9,11-hexadecadienal and optionally one or more precursors thereof.

43. The method of claim 40 to 42, wherein the genetically engineered yeast cell further expresses one or more enzymes selected from:
a. a Z11 desaturase catalyzing the formation of a double bond in a Z configuration in position 11 in the hexadecanoyl-CoA and thereby producing a (Z)-11-hexadecenoyl-CoA;
b. one or more alcohol-forming fatty acyl-CoA reductases (FAR) converting respectively the hexadecanoyl-CoA into hexadecan-1-ol, the (Z)-9-hexadecenoyl-CoA into (Z)-9-hexadecen-1-ol, and the (Z)-11-hexadecenoyl-CoA into (Z)-11-hexadecen-1-ol;
and the method further comprises enzymatically or chemically converting the hexadecan-1-ol into hexadecanal, the (Z)-9-hexadecen-1-ol into (Z)-9-hexadecenal and the (Z)-11-hexadecen-1-ol and the (Z)-11-hexadecen-1-ol into (Z)-11-hexadecenal; and/or optionally recovering and/or isolating the hexadecanal, the (Z)-9-hexadecenal and the (Z)-11-hexadecenal and optionally one or more precursors thereof.

44. The method of claim 40 to 43, further comprising mixing the recovered (Z,E)-9,11-hexadecadienal, (Z)-9-hexadecenal, (Z)-11-hexadecenal, hexadecanal and optionally one or more precursors thereof with one or more carriers, agents, additives, adjuvants and/or excipients to produce the biopesticide composition.

45. The method of claim 44, wherein the carriers, agents, additives, adjuvants and/or excipients comprise one or more compounds selected from
a) a protective agent comprising a conjugated sulfur compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione, which protects the target compound from from being converted into an acid; and/or
b) a carrier facilitating slow release of the (Z,E)-9,11-hexadecadienal, (Z)-9-hexadecenal, (Z)-11-hexadecenal and/or hexadecanal from the mixture, optionally being (i) a polymeric substrate selected from plastic, wax emulsion, oil emulsion, or microcapsules and/or (ii) a zeolite.

46. A biopesticide composition comprising a target compound selected from (Z,E)-9,11-hexadecadienal, and optionally one or more compounds selected from (Z)-9-hexadecenal, (Z)-11-hexadecenal and/or hexadecanal, in combination with one or more carriers, agents, additives, adjuvants and/or excipients.

47. The biopesticide composition of claim 46, further comprising at least trace amounts of one or more compounds selected from hexadecan-1-ol, (Z)-9-hexadecen-1-ol, (Z)-11-hexadecen-1-ol, and (Z,E)-9,11-hexadecadien-1-ol, and optionally other metabolites of the cell culture.

48. The biopesticide composition of claim 46 to 47, further comprising (Z,E)-9,11-hexadecandienyl acetate.

49. The biopesticide composition of claim 46 to 48 wherein one or more of the hexadecanoyl-CoA, (Z)-9-hexadecenoyl-CoA, (Z)-11-hexadecenoyl-CoA, (Z,E)-9,11-hexadecadienoyl-CoA, hexadecan-1-ol, (Z)-9-hexadecen-1-ol, (Z)-11-hexadecen-1-ol, and (Z,E)-9,11-hexadecadien-1-ol are obtained from the method of claim 26 to 41 and optionally the composition comprises one or more further compounds or metabolites from the cell culture of claim 25.

50. The biopesticide composition of claim 46 to 49, wherein the biopesticide composition comprises at least 20% biobased carbon, such as at least 30% biobased carbon, such as at least 40% biobased carbon, such as at least 50% biobased carbon, such as at least 60% biobased carbon, such as at least 70% biobased carbon, such as at least 75% biobased carbon, such as at least 80% biobased carbon, such as at least 85% biobased carbon, such as at least 90% biobased carbon, such as at least 95% biobased carbon, such as 100% biobased carbon.

51. The biopesticide composition of claim 46 to 49, wherein the biopesticide composition comprises from 20% to 100% biobased carbon, such as from 30% to 100% biobased carbon, such as from 40% to 100% biobased carbon, such as from 50% to 100% biobased carbon, such as from 60% to 100% biobased carbon, such as from 70% to 100% biobased carbon, such as from 75% to 100% biobased carbon, such as from 80% to 100% biobased carbon, such as from 85% to 100% biobased carbon, such as from 90% to 100% biobased carbon, such as from 95% to 100% biobased carbon, such as 100% biobased carbon.

52. The biopesticide composition of claim 46 to 49, wherein the composition comprises at least 50% biobased carbon, such as at least 55%, such as at least 60%, such as at least 65%, such as at least 70%, such as at least 75%, such as at least 80%, such as at least 85%, such as at least 90%, such as at least 95%, such as at least 99%, such as at least 100%.

53. The biopesticide composition of claim 46 to 49, wherein the composition comprises 90% biobased carbon, 91% biobased carbon, 92% biobased carbon, 93% biobased carbon, 94% biobased carbon, 95% biobased carbon, 96% biobased carbon, 97% biobased carbon, 98% biobased carbon, 99% biobased carbon, or 100% biobased carbon, for example 94% biobased carbon.

54. The biopesticide composition of claim 46 to 52, wherein the composition comprises no more than 50% fossil-based carbon, such as no more than 45%, such as no more than 40%, such as no more than 35%, such as no more than 30%, such as no more than 25%, such as no more than 20%, such as no more than 15%, such as no more than 10%, such as no more than 5%, such as no more than 1% fossil-based carbon.

55. The biopesticide composition of claim 46 to 54, wherein the concentration of the target compound is at least 1 mg/kg composition.

56. The biopesticide composition of claim 46 to 55, further comprising a protective agent comprising a conjugated sulfur, which protects the target compound from being degraded, such as degraded by conversion into an acid.

57. The biopesticide composition of claim 56, wherein the protective agent comprises a compound selected from zinc pyrithione, 5-amino-1,3,4-thiadiazole-2-thiol, 2-thiazoline-2-thiol, 5-methyl-1,3,4-thiadiazole-2-thiol, 2-mercapto-benzimidazole, 2-mercapto-1-methylimidazole and sodium pyrithione.

58. The biopesticide composition of claim 56 to 57, comprising at least 10 mg of the protective agent per gram aldehyde and/or alcohol.

59. The biopesticide composition of claim 46 to 58, further comprising a carrier facilitating slow release of the target compound, optionally being (i) a polymeric substrate selected from plastic, wax emulsion, oil emulsion, or microcapsules and/or (ii) zeolite.

60. A method of controlling or monitoring a pest comprising distributing the biopesticide composition of claim 46 to 59 in a habitat for the pest and allowing the target compound to control the pest.

61. The method of claim 60, wherein the habitat is a sugar cane field, and the pest is Diatraea saccharalis.
